SEQUENCE LISTING (26 sequences)

SEQ ID NO: 1 - protein, 901 aa, Streptococcus agalactiae serotype V strain 2603 V/R
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPMTGGKGILSFILIGGAMMSIAGGIYIWKRYKKSSDMSIKKD

SEQ ID NO: 2 - protein, 554 aa, Streptococcus agalactiae serotype V strain 2603 V/R
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 3 - protein, 502 aa, Streptococcus agalactiae serotype III strain COH1
MKKKMIQSLLVASLAFGMAVSPVTPIAFAAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTELPSTGGIGTTIFYIIGAILVIGAGIVLVARRRLRS

SEQ ID NO: 4 - protein, 869 aa, Artificial Sequence
  Amino acid sequence of GBS67 sequenced from Streptococcus agalactiae serotype V strain 2603 V/R truncated before C-terminal transmembrane region
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPMTGGKGILS

SEQ ID NO: 5 - protein, 858 aa, Artificial Sequence
  Amino acid sequence of GBS67 sequenced from Streptococcus agalactiae serotype V strain 2603 V/R lacking transmembrane and cell wall anchor motifs
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGI

SEQ ID NO: 6 - protein, 517 aa, Artificial Sequence
  Amino acid sequence of GBS80 sequenced from Streptococcus agalactiae serotype V strain 2603 V/R lacking the N-terminal leader or signal sequence region
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 7 - protein, 525 aa, Artificial Sequence
  Amino acid sequence of GBS80 sequenced from Streptococcus agalactiae serotype V strain 2603 V/R lacking the C-terminal transmembrane region and downstream amino acids
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTG

SEQ ID NO: 8 - protein, 520 aa, Artificial Sequence
  Amino acid sequence of GBS80 sequenced from Streptococcus agalactiae serotype V strain 2603 V/R lacking the transmembrane and cytoplasmic regions and the cell wall anchor motif
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 9 - protein, 483 aa, Artificial Sequence
  Amino acid sequence of GBS80 sequenced from Streptococcus agalactiae serotype V strain 2603 V/R lacking the leader or signal sequence region, the transmembrane and cytoplasmic regio
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 10 - protein, 271 aa, Artificial Sequence
  A particularly immunogenic fragment of GBS80 located towards the N-terminus of the protein
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKG

SEQ ID NO: 11 - protein, 473 aa, Artificial Sequence
  Amino acid sequence of SpbI from Streptococcus agalactiae serotype III strain COH1 lacking the N-terminal leader or signal sequence region
AETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTELPSTGGIGTTIFYIIGAILVIGAGIVLVARRRLRS

SEQ ID NO: 12 - protein, 467 aa, Artificial Sequence
  Amino acid sequence of SpbI from Streptococcus agalactiae serotype III strain COH1 lacking the cell wall anchor motif and sequence C-terminal to this motif
MKKKMIQSLLVASLAFGMAVSPVTPIAFAAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTE

SEQ ID NO: 13 - protein, 438 aa, Artificial Sequence
  Amino acid sequence of SpbI from Streptococcus agalactiae serotype III strain COH1 lacking the leader or signal sequence region, the cell wall anchor motif and sequence C terminal to this motif
AETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTE

SEQ ID NO: 14 - DNA, 12 bp, Artificial Sequence
  Nucleotide sequence upstream of internal methionine codon of wild-type SpbI, containing core Shine-Dalgarno sequence
taatggagctgt

SEQ ID NO: 15 - protein, 4 aa, Artificial Sequence
  Linker
GGGG

SEQ ID NO: 16 - protein, 14 aa, Artificial Sequence
  Linker
GGGGSGGGGSGGGG

SEQ ID NO: 17 - protein, 17 aa, Artificial Sequence
  Linker
GGGGSGGGGSGGGGSEL

SEQ ID NO: 18 - protein, 6 aa, Artificial Sequence
  Linker
GSGGGG

SEQ ID NO: 19 - protein, 3 aa, Artificial Sequence
  W moiety
MAS

SEQ ID NO: 20 - protein, 6 aa, Artificial Sequence
  Z moiety
HHHHHH

SEQ ID NO: 21 - protein, 941 aa, Artificial Sequence
  Combination of W, X, L and Y moieties
MASAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTEGGGGSGGGGSGGGGSELAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 22 - protein, 947 aa, Artificial Sequence
  Combination of W, X, L, Y and Z moieties
MASAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTEGGGGSGGGGSGGGGSELAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSHHHHHH

SEQ ID NO: 23 - protein, 932 aa, Artificial Sequence
  Combination of W, X, L and Y moieties
MASAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTEGSGGGGELAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 24 - protein, 896 aa, Streptococcus agalactiae serotype Ib strain H36B
MRKYQKFSKILTLSLFCLSQIPLNTNVLGESTVPENGAKGKLVVKKTDDQNKPLSKATFVLKPTSHSESKVEKVTTEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGKSIIKTINKDEPLDVVFVLDNSNSMKNNGKNNKAKKAGEAVETIIKDVLGANVENRAALVTYGSDIFDGRTVKVIKGFKEDPYYGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVKGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSDGEITELMNSFSSKPEYYTPIVTSADVSNNEILSKIQQQFEKILTKENSIVNGTIEDPMGDKINLHLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILSFILIGGAMMSIAGGIYIWKRHKKSSDASIEKD

SEQ ID NO: 25 - protein, 864 aa, Artificial Sequence
  Amino acid sequence of GBS67 sequenced from Streptococcus agalactiae serotype Ib strain H36B truncated before C-terminal transmembrane region
MRKYQKFSKILTLSLFCLSQIPLNTNVLGESTVPENGAKGKLVVKKTDDQNKPLSKATFVLKPTSHSESKVEKVTTEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGKSIIKTINKDEPLDVVFVLDNSNSMKNNGKNNKAKKAGEAVETIIKDVLGANVENRAALVTYGSDIFDGRTVKVIKGFKEDPYYGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVKGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSDGEITELMNSFSSKPEYYTPIVTSADVSNNEILSKIQQQFEKILTKENSIVNGTIEDPMGDKINLHLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILS

SEQ ID NO: 26 - protein, 853 aa, Artificial Sequence
  Amino acid sequence of GBS67 sequenced from Streptococcus agalactiae serotype Ib strain H36B lacking transmembrane and cell wall anchor motifs
MRKYQKFSKILTLSLFCLSQIPLNTNVLGESTVPENGAKGKLVVKKTDDQNKPLSKATFVLKPTSHSESKVEKVTTEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGKSIIKTINKDEPLDVVFVLDNSNSMKNNGKNNKAKKAGEAVETIIKDVLGANVENRAALVTYGSDIFDGRTVKVIKGFKEDPYYGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVKGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSDGEITELMNSFSSKPEYYTPIVTSADVSNNEILSKIQQQFEKILTKENSIVNGTIEDPMGDKINLHLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGI

(19) United States
(12) Patent Application Publication
(10) Pub. No.: US 2018/0036402 A1
(43) Pub. Date: Feb. 8, 2018

(54) IMMUNOGENIC COMPOSITIONS

(71) Applicant: GLAXOSMITHKLINE BIOLOGICALS SA, Rixensart (BE)

(72) Inventors: Francesco BERTI, Colle Val D'Elsa (IT); Mario CONTORNI, Siena (IT); Paolo COSTANTINO, Siena (IT); Oretta FINCO, Rapolano Terme (IT); Guido GRANDI, Milan (IT); Domenico MAIONE, Siena (IT); John TELFORD, Monteriggioni (IT)

(73) Assignee: GLAXOSMITHKLINE BIOLOGICALS SA, Rixensart (BE)

(21) Appl. No.: 15/705,873

(22) Filed: Sep. 15, 2017

Related U.S. Application Data
(62) Division of application No. 14/662,429, filed on Mar. 19, 2015.
Application No. 14/662,429 is a continuation of application No. 13/824,041, filed on Jun. 10, 2013, now abandoned.
(60) Provisional application No. 61/383,668, filed on Sep. 16, 2010.

(30) Foreign Application Priority Data
Jan. 31, 2011 (GB) 1101665.6

(51) Int. Cl. A61K 39/385; A61K 39/09; A61K 39/00

(52) U.S. Cl. CPC A61K 39/385; A61K 39/092; A61K 47/646; A61K 2039/627; A61K 2039/6037

(57) ABSTRACT

A method of immunization against Group B Streptococcus infection, using an immunogenic composition conjugates of GBS capsular saccharide conjugated to carrier proteins.

This Application is filed pursuant to 35 U.S.C. § 111(a) as a U.S. Divisional Application which claims priority from U.S. application Ser. No. 14/662,429, filed Mar. 19, 2015 as a Continuation Application of U.S. application Ser. No. 13/824,041, filed Jun. 10, 2013 pursuant to 35 U.S.C. § 371 as a U.S. National Phase Application of International Patent Application Serial No. PCT/IB2011/054069, filed Sep. 16, 2011, which claims priority to U.S. Provisional Application Ser. No. 61/383,668, filed on Sep. 16, 2010; and to United Kingdom Patent Application Serial No. 1101665.6, filed Jan. 31, 2011, and the entire contents of each of the foregoing applications are hereby incorporated by reference.

This application incorporates by reference the contents of 1 108 kb text file created on Mar. 18, 2015 and named “PAT052612_US_CNT sequencelisting.txt,” which is the sequence listing for this application.

### TECHNICAL FIELD

This invention is in the field of immunogenic compositions comprising conjugates of Streptococcus agalactiae capsular saccharides and carrier proteins. The compositions are useful for immunisation.

### BACKGROUND ART

The capsular saccharides of bacteria have been used for many years in vaccines against capsulated bacteria. As saccharides are T-independent antigens, however, they are poorly immunogenic. Conjugation to a carrier can convert T-independent antigens into T-dependent antigens, thereby enhancing memory responses and allowing protective immunity to develop. The most effective saccharide vaccines are therefore based on glycoconjugates, and the prototype conjugate vaccine was against Haemophilus influenzae type b (‘Hib’) [e.g. see chapter 14 of ref. Error! Bookmark not defined.].

Another bacterium for which conjugate vaccines have been described is Streptococcus agalactiae, also known as ‘group B streptococcus’, or simply as ‘GBS’. Much of this work has been performed by Dennis Kasper and colleagues, and is described in documents such as references 1 to 9. Conjugate vaccines for each of GBS serotypes Ia, Ib, II, III, and V have been shown to be safe and immunogenic in humans [10]. However, there remains a need for further and improved GBS conjugate vaccines.

### BRIEF DESCRIPTION OF THE DRAWINGS

FIG. 1 shows the difference between the repeating structures in GBS serotypes Ia and III.

FIG. 2 shows the repeating structures of capsular saccharides in GBS serotypes Ia, Ib, II, III & V.

FIG. 3 shows the repeating structure of the desialylated form of the capsular saccharide from GBS serotype V.

FIG. 4 shows the effect of priming with CRM197 prior to administration of a conjugate of GBS serotype III capsular saccharide and CRM197, with and without adjuvant.

### DETAILED DESCRIPTION

In a first aspect, the invention makes use of one or more conjugates that are capsular saccharides from GBS serotypes Ia, Ib, III or V conjugated to a carrier protein. In particular, the invention provides immunogenic compositions comprising one or more of these conjugates. The compositions may be used as vaccines for preventing infection by these GBS serotype(s).

In a second aspect, the invention provides a method for immunising a patient against infection by GBS comprising the step of administering to the patient a conjugate that is a capsular saccharide from GBS conjugated to a diphtheria toxoid or derivative thereof, wherein the patient has been pre-immunised with a diphtheria toxoid or derivative thereof. Typically, the conjugate is one of the GBS conjugates in an immunogenic composition of the first aspect of the invention.

Immunogenic Compositions

In one embodiment, the invention provides an immunogenic composition comprising a conjugate that is a capsular saccharide from GBS serotype Ia conjugated to a carrier protein. In a second embodiment, the invention provides an immunogenic composition comprising a conjugate that is a capsular saccharide from GBS serotype Ib conjugated to a carrier protein. In a third embodiment, the invention provides an immunogenic composition comprising a conjugate that is a capsular saccharide from GBS serotype III conjugated to a carrier protein. In a fourth embodiment, the invention provides an immunogenic composition comprising a conjugate that is a capsular saccharide from GBS serotype V conjugated to a carrier protein.

The immunogenic compositions may comprise more than one conjugate. Embodiments of the invention comprising two, three or four conjugates are described below. Of these compositions, the inventors have found that compositions comprising a conjugate that is a capsular saccharide from GBS serotype Ib conjugated to a carrier protein may confer protection against GBS serotype Ia in addition to GBS serotype Ib. This observation is in contrast to the teaching of reference 11, which suggests that type Ib conjugates are not capable of inducing antibodies that can kill type Ia bacteria. Accordingly, the embodiments described below that comprise a conjugate that is a capsular saccharide from GBS serotype Ib conjugated to a carrier protein may be advantageous in that they provide enhanced protection against serotype Ia (when the composition also comprises a conjugate that is a capsular saccharide from GBS serotype Ia conjugated to a carrier protein), and may even provide protection when the composition does not comprise a conjugate that is a capsular saccharide from GBS serotype Ia conjugated to a carrier protein.

As described above, the immunogenic compositions may comprise two conjugates. In one embodiment, the first conjugate is a capsular saccharide from GBS serotype Ia conjugated to a carrier protein, while the second conjugate is a capsular saccharide from GBS serotype Ib conjugated to a carrier protein. In a second embodiment, the first conjugate is a capsular saccharide from GBS serotype Ia conjugated to a carrier protein, while the second conjugate is a capsular saccharide from GBS serotype III conjugated to a carrier protein. In a third embodiment, the first conjugate is a capsular saccharide from GBS serotype Ia conjugated to a carrier protein, while the second conjugate is a capsular saccharide from GBS serotype V conjugated to a carrier protein. In a fourth embodiment, the first conjugate is a capsular saccharide from GBS serotype Ib conjugated to a carrier protein, while the second conjugate is a capsular saccharide from GBS serotype III conjugated to a carrier protein. In a fifth embodiment, the first conjugate is a capsular saccharide from GBS serotype Ib conjugated to a carrier protein, while the second conjugate is a capsular saccharide from GBS serotype V conjugated to a carrier protein. In a sixth embodiment, the first conjugate is a capsular saccharide from GBS serotype III conjugated to a carrier protein, while the second conjugate is a capsular saccharide from GBS serotype V conjugated to a carrier protein.

Similarly, the immunogenic compositions may comprise three conjugates. In one embodiment, the first conjugate is a capsular saccharide from GBS serotype Ia conjugated to a carrier protein, while the second conjugate is a capsular saccharide from GBS serotype Ib conjugated to a carrier protein and the third conjugate is a capsular saccharide from GBS serotype III conjugated to a carrier protein. The inventors have found that such compositions (e.g. as exemplified below) are particularly suitable for use as vaccines to prevent infection by GBS. This embodiment is therefore a preferred embodiment of the invention. In a second embodiment, the first conjugate is a capsular saccharide from GBS serotype Ia conjugated to a carrier protein, while the second conjugate is a capsular saccharide from GBS serotype Ib conjugated to a carrier protein and the third conjugate is a capsular saccharide from GBS serotype V conjugated to a carrier protein. In a third embodiment, the first conjugate is a capsular saccharide from GBS serotype Ia conjugated to a carrier protein, while the second conjugate is a capsular saccharide from GBS serotype III conjugated to a carrier protein and the third conjugate is a capsular saccharide from GBS serotype V conjugated to a carrier protein. In a fourth embodiment, the first conjugate is a capsular saccharide from GBS serotype Ib conjugated to a carrier protein, while the second conjugate is a capsular saccharide from GBS serotype III conjugated to a carrier protein and the third conjugate is a capsular saccharide from GBS serotype V conjugated to a carrier protein.

In the same way, the immunogenic compositions may comprise four conjugates. In one embodiment, the first conjugate is a capsular saccharide from GBS serotype Ia conjugated to a carrier protein, while the second conjugate is a capsular saccharide from GBS serotype Ib conjugated to a carrier protein, the third conjugate is a capsular saccharide from GBS serotype III conjugated to a carrier protein and the fourth conjugate is a capsular saccharide from GBS serotype V conjugated to a carrier protein.

Typically, the immunogenic compositions described above will not comprise any conjugates other than those specifically mentioned, particularly conjugates comprising capsular saccharides from GBS serotypes other than those specifically mentioned. However, in some embodiments, the compositions may comprise other conjugates, including conjugates comprising capsular saccharides from other GBS serotypes. For example, the compositions may comprise a conjugate that is a capsular saccharide from GBS serotype II conjugated to a carrier protein. Similarly, the compositions may comprise a conjugate that is a capsular saccharide from GBS serotype VI conjugated to a carrier protein. In another possibility, the compositions may comprise a conjugate that is a capsular saccharide from GBS serotype VIII conjugated to a carrier protein.

The immunogenic compositions described above may comprise any suitable amount of the capsular saccharide(s) per unit dose. Suitable amounts of the capsular saccharide(s) may be from 0.1 to 50 μg per unit dose. Typically, each GBS capsular saccharide is present at an amount from 1 to 30 μg, for example from 2 to 25 μg, and in particular from 5 to 20 μm. Suitable amounts of the capsular saccharide(s) may include 5, 10 and 20 μg per unit dose. The inventors have found that these amounts are suitable, particularly when the immunogenic composition comprises capsular saccharides from GBS serotypes Ia, Ib and/or III. Suitable amounts per unit dose of each capsular saccharide in the embodiments described above may therefore be selected from the numbered options in the following tables, wherein the relevant embodiment is indicated by reference to the serotype(s) from which the capsular saccharide(s) in the composition are derived:

TABLE A
Immunogenic compositions comprising one conjugate

| | |
|---|---|
| Dosing | Embodiment |

| option | Ia | Ib | III | V |
|---|---|---|---|---|
| 1 | Ia: 5 μg | Ib: 5 μg | III: 5 μg | V: 5 μg |
| 2 | Ia: 10 μg | Ib: 10 μg | III: 10 μg | V: 10 μg |
| 3 | Ia: 20 μg | Ib: 20 μg | III: 20 μg | V: 20 μg |

TABLE B
Immunogenic compositions comprising two conjugates

| Dosing | Embodiment | | | | | |
|---|---|---|---|---|---|---|
| option | Ia & Ib | Ia & III | Ia & V | Ib & III | Ib & V | III & V |
| 1 | Ia: 5 μg | Ia: 5 μg | Ia: 5 μg | Ib: 5 μg | Ib: 5 μg | III: 5 μg |
| | Ib: 5 μg | III: 5 μg | V: 5 μg | III: 5 μg | V: 5 μg | V: 5 μg |
| 2 | Ia: 5 μg | Ia: 5 μg | Ia: 5 μg | Ib: 5 μg | Ib: 5 μg | III: 5 μg |
| | Ib: 10 μg | III: 10 μg | V: 10 μg | III: 10 μg | V: 10 μg | V: 10 μg |
| 3 | Ia: 5 μg | Ia: 5 μg | Ia: 5 μg | Ib: 5 μg | Ib: 5 μg | III: 5 μg |
| | Ib: 20 μg | III: 20 μg | V: 20 μg | III: 20 μg | V: 20 μg | V: 20 μg |
| 4 | Ia: 10 μg | Ia: 10 μg | Ia: 10 μg | Ib: 10 μg | Ib: 10 μg | III: 10 μg |
| | Ib: 5 μg | III: 5 μg | Ib: 5 μg | III: 5 μg | V: 5 μg | V: 5 μg |
| 5 | Ia: 10 μg | Ia: 10 μg | Ia: 10 μg | Ib: 10 μg | Ib: 10 μg | III: 10 μg |
| | Ib: 10 μg | III: 10 μg | V: 10 μg | III: 10 μg | V: 10 μg | V: 10 μg |
| 6 | Ia: 10 μg | Ia: 10 μg | Ia: 10 μg | Ib: 10 μg | Ib: 10 μg | III: 10 μg |
| | Ib: 20 μg | III: 20 μg | V: 20 μg | III: 20 μg | V: 20 μg | V: 20 μg |
| 7 | Ia: 20 μg | Ia: 20 μg | Ia: 20 μg | Ib: 20 μg | Ib: 20 μg | III: 20 μg |
| | Ib: 5 μg | III: 5 μg | V: 5 μg | III: 5 μg | V: 5 μg | V: 5 μg |
| 8 | Ia: 20 μg | Ia: 20 μg | Ia: 20 μg | Ib: 20 μg | Ib: 20 μg | III: 20 μg |
| | Ib: 10 μg | III: 10 μg | V: 10 μg | III: 10 μg | V: 10 μg | V: 10 μg |
| 9 | Ia: 20 μg | Ia: 20 μg | Ia: 20 μg | Ib: 20 μg | Ib: 20 μg | III: 20 μg |
| | Ib: 20 μg | III: 20 μg | V: 20 μg | III: 20 μg | V: 20 μg | V: 20 μg |

TABLE C
Immunogenic compositions comprising three conjugates

| Dosing | Embodiment | | | |
|---|---|---|---|---|
| option | Ia, Ib & III | Ia, Ib & V | Ia, III & V | Ib, III & V |
| 1 | Ia: 5 μg | Ia: 5 μg | Ia: 5 μg | Ib: 5 μg |
| | Ib: 5 μg | Ib: 5 μg | III: 5 μg | III: 5 μg |
| | III: 5 μg | V: 5 μg | V: 5 μg | V: 5 μg |
| 2 | Ia: 5 μg | Ia: 5 μg | Ia: 5 μg | Ib: 5 μg |
| | Ib: 5 μg | Ib: 5 μg | III: 5 μg | III: 5 μg |
| | III: 10 μg | V: 10 μg | V: 10 μg | V: 10 μg |
| 3 | Ia: 5 μg | Ia: 5 μg | Ia: 5 μg | Ib: 5 μg |
| | Ib: 5 μg | Ib: 5 μg | III: 5 μg | III: 5 μg |
| | III: 20 μg | V: 20 μg | V: 20 μg | V: 20 μg |
| 4 | Ia: 5 μg | Ia: 5 μg | Ia: 5 μg | Ib: 5 μg |
| | Ib: 10 μg | Ib: 10 μg | III: 10 μg | III: 10 μg |
| | III: 5 μg | V: 5 μg | V: 5 μg | V: 5 μg |
| 5 | Ia: 5 μg | Ia: 5 μg | Ia: 5 μg | Ib: 5 μg |
| | Ib: 10 μg | Ib: 10 μg | III: 10 μg | III: 10 μg |
| | III: 10 μg | V: 10 μg | V: 10 μg | V: 10 μg |
| 6 | Ia: 5 μg | Ia: 5 μg | Ia: 5 μg | Ib: 5 μg |
| | Ib: 10 μg | Ib: 10 μg | III: 10 μg | III: 10 μg |
| | III: 20 μg | V: 20 μg | V: 20 μg | V: 20 μg |
| 7 | Ia: 5 μg | Ia: 5 μg | Ia: 5 μg | Ib: 5 μg |
| | Ib: 20 μg | Ib: 20 μg | III: 20 μg | III: 20 μg |
| | III: 5 μg | V: 5 μg | V: 5 μg | V: 5 μg |
| 8 | Ia: 5 μg | Ia: 5 μg | Ia: 5 μg | Ib: 5 μg |
| | Ib: 20 μg | Ib: 20 μg | III: 20 μg | III: 20 μg |
| | III: 10 μg | V: 10 μg | V: 10 μg | V: 10 μg |
| 9 | Ia: 5 μg | Ia: 5 μg | Ia: 5 μg | Ib: 5 μg |
| | Ib: 20 μg | Ib: 20 μg | III: 20 μg | III: 20 μg |
| | III: 20 μg | V: 20 μg | V: 20 μg | V: 20 μg |
| 10 | Ia: 10 μg | Ia: 10 μg | Ia: 10 μg | Ib: 10 μg |
| | Ib: 5 μg | Ib: 5 μg | III: 5 μg | III: 5 μg |
| | III: 5 μg | V: 5 μg | V: 5 μg | V: 5 μg |
| 11 | Ia: 10 μg | Ia: 10 μg | Ia: 10 μg | Ib: 10 μg |
| | Ib: 5 μg | Ib: 5 μg | III: 5 μg | III: 5 μg |
| | III: 10 μg | V: 10 μg | V: 10 μg | V: 10 μg |
| 12 | Ia: 10 μg | Ia: 10 μg | Ia: 10 μg | Ib: 10 μg |
| | Ib: 5 μg | Ib: 5 μg | III: 5 μg | III: 5 μg |
| | III: 20 μg | V: 20 μg | V: 20 μg | V: 20 μg |
| 13 | Ia: 10 μg | Ia: 10 μg | Ia: 10 μg | Ib: 10 μg |
| | Ib: 10 μg | Ib: 10 μg | III: 10 μg | III: 10 μg |
| | III: 5 μg | V: 5 μg | V: 5 μg | V: 5 μg |
| 14 | Ia: 10 μg | Ia: 10 μg | Ia: 10 μg | Ib: 10 μg |
| | Ib: 10 μg | Ib: 10 μg | III: 10 μg | III: 10 μg |
| | III: 10 μg | V: 10 μg | V: 10 μg | V: 10 μg |
| 15 | Ia: 10 μg | Ia: 10 μg | Ia: 10 μg | Ib: 10 μg |
| | Ib: 10 μg | Ib: 10 μg | III: 10 μg | III: 10 μg |
| | III: 20 μg | V: 20 μg | V: 20 μg | V: 20 μg |
| 16 | Ia: 10 μg | Ia: 10 μg | Ia: 10 μg | Ib: 10 μg |
| | Ib: 20 μg | Ib: 20 μg | III: 20 μg | III: 20 μg |
| | III: 5 μg | V: 5 μg | V: 5 μg | V: 5 μg |
| 17 | Ia: 10 μg | Ia: 10 μg | Ia: 10 μg | Ib: 10 μg |
| | Ib: 20 μg | Ib: 20 μg | III: 20 μg | III: 20 μg |
| | III: 10 μg | V: 10 μg | V: 10 μg | V: 10 μg |
| 18 | Ia: 10 μg | Ia: 10 μg | Ia: 10 μg | Ib: 10 μg |
| | Ib: 20 μg | Ib: 20 μg | III: 20 μg | III: 20 μg |
| | III: 20 μg | V: 20 μg | V: 20 μg | V: 20 μg |
| 19 | Ia: 20 μg | Ia: 20 μg | Ia: 20 μg | Ib: 20 μg |
| | Ib: 5 μg | Ib: 5 μg | III: 5 μg | III: 5 μg |
| | III: 5 μg | V: 5 μg | V: 5 μg | V: 5 μg |
| 20 | Ia: 20 μg | Ia: 20 μg | Ia: 20 μg | Ib: 20 μg |
| | Ib: 5 μg | Ib: 5 μg | III: 5 μg | III: 5 μg |
| | III: 10 μg | V: 10 μg | V: 10 μg | V: 10 μg |
| 21 | Ia: 20 μg | Ia: 20 μg | Ia: 20 μg | Ib: 20 μg |
| | Ib: 5 μg | Ib: 5 μg | III: 5 μg | III: 5 μg |
| | III: 20 μg | V: 20 μg | V: 20 μg | V: 20 μg |
| 22 | Ia: 20 μg | Ia: 20 μg | Ia: 20 μg | Ib: 20 μg |
| | Ib: 10 μg | Ib: 10 μg | III: 10 μg | III: 10 μg |
| | III: 5 μg | V: 5 μg | V: 5 μg | V: 5 μg |
| 23 | Ia: 20 μg | Ia: 20 μg | Ia: 20 μg | Ib: 20 μg |
| | Ib: 10 μg | Ib: 10 μg | III: 10 μg | III: 10 μg |
| | III: 10 μg | V: 10 μg | V: 10 μg | V: 10 μg |
| 24 | Ia: 20 μg | Ia: 20 μg | Ia: 20 μg | Ib: 20 μg |
| | Ib: 10 μg | Ib: 10 μg | III: 10 μg | III: 10 μg |
| | III: 20 μg | V: 20 μg | V: 20 μg | V: 20 μg |
| 25 | Ia: 20 μg | Ia: 20 μg | Ia: 20 μg | Ib: 20 μg |
| | Ib: 20 μg | Ib: 20 μg | III: 20 μg | III: 20 μg |
| | III: 5 μg | V: 5 μg | V: 5 μg | V: 5 μg |
| 26 | Ia: 20 μg | Ia: 20 μg | Ia: 20 μg | Ib: 20 μg |
| | Ib: 20 μg | Ib: 20 μg | III: 20 μg | III: 20 μg |
| | III: 10 μg | V: 10 μg | V: 10 μg | V: 10 μg |
| 27 | Ia: 20 μg | Ia: 20 μg | Ia: 20 μg | Ib: 20 μg |
| | Ib: 20 μg | Ib: 20 μg | III: 20 μg | III: 20 μg |
| | III: 20 μg | V: 20 μg | V: 20 μg | V: 20 μg |

Of the dosing options described in Table C, the inventors have found that options 1, 14 and 27 are effective, particularly when the immunogenic composition comprises: a) a conjugate that is a capsular saccharide from GBS serotype Ia conjugated to a carrier protein; b) a conjugate that is a capsular saccharide from GBS serotype Ib conjugated to a carrier protein; and c) a conjugate that is a capsular saccharide from GBS serotype III conjugated to a carrier protein. These dosing options are therefore preferred for use in the invention, particularly for this embodiment. It may be advantageous to minimise the total amount of capsular saccharide(s) per unit dose in order to reduce potential toxicity. Accordingly, dosing option 1 is particularly preferred.

It may be possible to further minimise the amount of capsular saccharide(s) per unit dose. In particular, suitable amounts of the capsular saccharide(s) may be from 0.1 to 5 μg per unit dose. Typically, each GBS capsular saccharide may therefore be present at an amount from 0.1 to 5 μg, e.g. 0.5, 2.5 or 5 μg, per unit dose. For example, each GBS capsular saccharide may be present at an amount from 0.5 to 5 μg, 1 to 4 μg, 2 to 3 μg, or about 2.5 μg per unit dose. The inventors envisage that these amounts will be suitable, particularly when the immunogenic composition comprises a) a conjugate that is a capsular saccharide from GBS serotype Ia conjugated to a carrier protein; b) a conjugate that is a capsular saccharide from GBS serotype Ib conjugated to a carrier protein; and c) a conjugate that is a capsular saccharide from GBS serotype III conjugated to a carrier protein. Suitable amounts per unit dose of each capsular saccharide in this embodiment may therefore be selected from the numbered options in the table below:

TABLE C′
Immunogenic compositions comprising capsular
saccharides from GBS serotypes Ia, Ib and III

| | Amount of capsular saccharide | | |
|---|---|---|---|
| Dosing | per unit dose (μg) | | |
| option | Ia | Ib | III |
| 1 | 0.5 | 0.5 | 0.5 |
| 2 | 0.5 | 0.5 | 2.5 |
| 3 | 0.5 | 0.5 | 5 |
| 4 | 0.5 | 2.5 | 0.5 |
| 5 | 0.5 | 2.5 | 2.5 |
| 6 | 0.5 | 2.5 | 5 |
| 7 | 0.5 | 5 | 0.5 |
| 8 | 0.5 | 5 | 2.5 |
| 9 | 0.5 | 5 | 5 |
| 10 | 2.5 | 0.5 | 0.5 |
| 11 | 2.5 | 0.5 | 2.5 |
| 12 | 2.5 | 0.5 | 5 |
| 13 | 2.5 | 2.5 | 0.5 |
| 14 | 2.5 | 2.5 | 2.5 |
| 15 | 2.5 | 2.5 | 5 |
| 16 | 2.5 | 5 | 0.5 |
| 17 | 2.5 | 5 | 2.5 |
| 18 | 2.5 | 5 | 5 |
| 19 | 5 | 0.5 | 0.5 |
| 20 | 5 | 0.5 | 2.5 |
| 21 | 5 | 0.5 | 5 |
| 22 | 5 | 2.5 | 0.5 |
| 23 | 5 | 2.5 | 2.5 |
| 24 | 5 | 2.5 | 5 |
| 25 | 5 | 5 | 0.5 |
| 26 | 5 | 5 | 2.5 |
| 27 | 5 | 5 | 5 |

Of the dosing options described in Table C, the inventors particulary envisage options 1, 14 and 27. In these options, the amount of each GBS capsular saccharide is the same (e.g. as in the higher dose compositions exemplified below).

TABLE D
Immunogenic compositions comprising four conjugates
Embodiment - Ia, Ib, III & V

| Dosing option | |
|---|---|
| 1 | Ia: 5 μg |
| | Ib: 5 μg |
| | III: 5 μg |
| | V: 5 μg |
| 2 | Ia: 5 μg |
| | Ib: 5 μg |
| | III: 5 μg |
| | V: 10 μg |
| 3 | Ia: 5 μg |
| | Ib: 5 μg |
| | III: 5 μg |
| | V: 20 μg |
| 4 | Ia: 5 μg |
| | Ib: 5 μg |
| | III: 10 μg |
| | V: 5 μg |
| 5 | Ia: 5 μg |
| | Ib: 5 μg |
| | III: 10 μg |
| | V: 10 μg |
| 6 | Ia: 5 μg |
| | Ib: 5 μg |
| | III: 10 μg |
| | V: 20 μg |
| 7 | Ia: 5 μg |
| | Ib: 5 μg |
| | III: 20 μg |
| | V: 5 μg |
| 8 | Ia: 5 μg |
| | Ib: 5 μg |
| | III: 20 μg |
| | V: 10 μg |
| 9 | Ia: 5 μg |
| | Ib: 5 μg |
| | III: 20 μg |
| | V: 20 μg |
| 10 | Ia: 5 μg |
| | Ib: 10 μg |
| | III: 5 μg |
| | V: 5 μg |
| 11 | Ia: 5 μg |
| | Ib: 10 μg |
| | III: 5 μg |
| | V: 10 μg |
| 12 | Ia: 5 μg |
| | Ib: 10 μg |
| | III: 5 μg |
| | V: 20 μg |
| 13 | Ia: 5 μg |
| | Ib: 10 μg |
| | III: 10 μg |
| | V: 5 μg |
| 14 | Ia: 5 μg |
| | Ib: 10 μg |
| | III: 10 μg |
| | V: 10 μg |
| 15 | Ia: 5 μg |
| | Ib: 10 μg |
| | III: 10 μg |
| | V: 20 μg |
| 16 | Ia: 5 μg |
| | Ib: 10 μg |
| | III: 20 μg |
| | V: 5 μg |
| 17 | Ia: 5 μg |
| | Ib: 10 μg |
| | III: 20 μg |
| | V: 10 μg |
| 18 | Ia: 5 μg |
| | Ib: 10 μg |
| | III: 20 μg |
| | V: 20 μg |
| 19 | Ia: 5 μg |
| | Ib: 20 μg |
| | III: 5 μg |
| | V: 5 μg |
| 20 | Ia: 5 μg |
| | Ib: 20 μg |
| | III: 5 μg |
| | V: 10 μg |
| 21 | Ia: 5 μg |
| | Ib: 20 μg |
| | III: 5 μg |
| | V: 20 μg |
| 22 | Ia: 5 μg |
| | Ib: 20 μg |
| | III: 10 μg |
| | V: 5 μg |
| 23 | Ia: 5 μg |
| | Ib: 20 μg |
| | III: 10 μg |
| | V: 10 μg |
| 24 | Ia: 5 μg |
| | Ib: 20 μg |
| | III: 10 μg |
| | V: 20 μg |
| 25 | Ia: 5 μg |
| | Ib: 20 μg |
| | III: 20 μg |
| | V: 5 μg |
| 26 | Ia: 5 μg |
| | Ib: 20 μg |
| | III: 20 μg |
| | V: 10 μg |
| 27 | Ia: 5 μg |
| | Ib: 20 μg |
| | III: 20 μg |
| | V: 20 μg |
| 28 | Ia: 10 μg |
| | Ib: 5 μg |
| | III: 5 μg |
| | V: 5 μg |
| 29 | Ia: 10 μg |
| | Ib: 5 μg |
| | III: 5 μg |
| | V: 10 μg |
| 30 | Ia: 10 μg |
| | Ib: 5 μg |
| | III: 5 μg |
| | V: 20 μg |
| 31 | Ia: 10 μg |
| | Ib: 5 μg |
| | III: 10 μg |
| | V: 5 μg |
| 32 | Ia: 10 μg |
| | Ib: 5 μg |
| | III: 10 μg |
| | V: 10 μg |
| 33 | Ia: 10 μg |
| | Ib: 5 μg |
| | III: 10 μg |
| | V: 20 μg |
| 34 | Ia: 10 μg |
| | Ib: 5 μg |
| | III: 20 μg |
| | V: 5 μg |
| 35 | Ia: 10 μg |
| | Ib: 5 μg |
| | III: 20 μg |
| | V: 10 μg |
| 36 | Ia: 10 μg |
| | Ib: 5 μg |
| | III: 20 μg |
| | V: 20 μg |
| 37 | Ia: 10 μg |
| | Ib: 10 μg |
| | III: 5 μg |
| | V: 5 μg |
| 38 | Ia: 10 μg |
| | Ib: 10 μg |
| | III: 5 μg |
| | V: 10 μg |
| 39 | Ia: 10 μg |
| | Ib: 10 μg |
| | III: 5 μg |
| | V: 20 μg |
| 40 | Ia: 10 μg |
| | Ib: 10 μg |
| | III: 10 μg |
| | V: 5 μg |
| 41 | Ia: 10 μg |
| | Ib: 10 μg |
| | III: 10 μg |
| | V: 10 μg |
| 42 | Ia: 10 μg |
| | Ib: 10 μg |
| | III: 10 μg |
| | V: 20 μg |
| 43 | Ia: 10 μg |
| | Ib: 10 μg |
| | III: 20 μg |
| | V: 5 μg |
| 44 | Ia: 10 μg |
| | Ib: 10 μg |
| | III: 20 μg |
| | V: 10 μg |
| 45 | Ia: 10 μg |
| | Ib: 10 μg |
| | III: 20 μg |
| | V: 20 μg |
| 46 | Ia: 10 μg |
| | Ib: 20 μg |
| | III: 5 μg |
| | V: 5 μg |
| 47 | Ia: 10 μg |
| | Ib: 20 μg |
| | III: 5 μg |
| | V: 10 μg |
| 48 | Ia: 10 μg |
| | Ib: 20 μg |
| | III: 5 μg |
| | V: 20 μg |
| 49 | Ia: 10 μg |
| | Ib: 20 μg |
| | III: 10 μg |
| | V: 5 μg |
| 50 | Ia: 10 μg |
| | Ib: 20 μg |
| | III: 10 μg |
| | V: 10 μg |
| 51 | Ia: 10 μg |
| | Ib: 20 μg |
| | III: 10 μg |
| | V: 20 μg |
| 52 | Ia: 10 μg |
| | Ib: 20 μg |
| | III: 20 μg |
| | V: 5 μg |
| 53 | Ia: 10 μg |
| | Ib: 20 μg |
| | III: 20 μg |
| | V: 10 μg |
| 54 | Ia: 10 μg |
| | Ib: 20 μg |
| | III: 20 μg |
| | V: 20 μg |
| 55 | Ia: 20 μg |
| | Ib: 5 μg |
| | III: 5 μg |
| | V: 5 μg |
| 56 | Ia: 20 μg |
| | Ib: 5 μg |
| | III: 5 μg |
| | V: 10 μg |
| 57 | Ia: 20 μg |
| | Ib: 5 μg |
| | III: 5 μg |
| | V: 20 μg |
| 58 | Ia: 20 μg |
| | Ib: 5 μg |
| | III: 10 μg |
| | V: 5 μg |
| 59 | Ia: 20 μg |
| | Ib: 5 μg |
| | III: 10 μg |
| | V: 10 μg |
| 60 | Ia: 20 μg |
| | Ib: 5 μg |
| | III: 10 μg |
| | V: 20 μg |
| 61 | Ia: 20 μg |
| | Ib: 5 μg |
| | III: 20 μg |
| | V: 5 μg |
| 62 | Ia: 20 μg |
| | Ib: 5 μg |
| | III: 20 μg |
| | V: 10 μg |
| 63 | Ia: 20 μg |
| | Ib: 5 μg |
| | III: 20 μg |
| | V: 20 μg |
| 64 | Ia: 20 μg |
| | Ib: 10 μg |
| | III: 5 μg |
| | V: 5 μg |
| 65 | Ia: 20 μg |
| | Ib: 10 μg |
| | III: 5 μg |
| | V: 10 μg |
| 66 | Ia: 20 μg |
| | Ib: 10 μg |
| | III: 5 μg |
| | V: 20 μg |
| 67 | Ia: 20 μg |
| | Ib: 10 μg |
| | III: 10 μg |
| | V: 5 μg |
| 68 | Ia: 20 μg |
| | Ib: 10 μg |
| | III: 10 μg |
| | V: 10 μg |
| 69 | Ia: 20 μg |
| | Ib: 10 μg |
| | III: 10 μg |
| | V: 20 μg |
| 70 | Ia: 20 μg |
| | Ib: 10 μg |
| | III: 20 μg |
| | V: 5 μg |
| 71 | Ia: 20 μg |
| | Ib: 10 μg |
| | III: 20 μg |
| | V: 10 μg |
| 72 | Ia: 20 μg |
| | Ib: 10 μg |
| | III: 20 μg |
| | V: 20 μg |
| 73 | Ia: 20 μg |
| | Ib: 20 μg |
| | III: 5 μg |
| | V: 5 μg |
| 74 | Ia: 20 μg |
| | Ib: 20 μg |
| | III: 5 μg |
| | V: 10 μg |
| 75 | Ia: 20 μg |
| | Ib: 20 μg |
| | III: 5 μg |
| | V: 20 μg |
| 76 | Ia: 20 μg |
| | Ib: 20 μg |
| | III: 10 μg |
| | V: 5 μg |
| 77 | Ia: 20 μg |
| | Ib: 20 μg |
| | III: 10 μg |
| | V: 10 μg |
| 78 | Ia: 20 μg |
| | Ib: 20 μg |
| | III: 10 μg |
| | V: 20 μg |
| 79 | Ia: 20 μg |
| | Ib: 20 μg |
| | III: 20 μg |
| | V: 5 μg |
| 80 | Ia: 20 μg |
| | Ib: 20 μg |
| | III: 20 μg |
| | V: 10 μg |
| 81 | Ia: 20 μg |
| | Ib: 20 μg |
| | III: 20 μg |
| | V: 20 μg |

In the embodiments described above wherein the immunogenic composition comprises more than one conjugate, the ratio of the mass of a given capsular saccharide to the mass of the other capsular saccharide(s) may vary. Suitable ratios (w/w) for each capsular saccharide in the embodiments described above may therefore be selected from the numbered options in the following tables, wherein the relevant embodiment is indicated by reference to the serotype(s) from which the capsular saccharide(s) in the composition are derived:

TABLE E
Immunogenic compositions comprising two conjugates

| | |
|---|---|
| Ratio | Embodiment |

| option | Ia:Ib | Ia:III | Ia:V | Ib:III | Ib:V | III:V |
|---|---|---|---|---|---|---|
| 1 | 1:1 | 1:1 | 1:1 | 1:1 | 1:1 | 1:1 |
| 2 | 1:2 | 1:2 | 1:2 | 1:2 | 1:2 | 1:2 |
| 3 | 1:4 | 1:4 | 1:4 | 1:4 | 1:4 | 1:4 |
| 4 | 2:1 | 2:1 | 2:1 | 2:1 | 2:1 | 2:1 |
| 7 | 4:1 | 4:1 | 4:1 | 4:1 | 4:1 | 4:1 |

TABLE F
Immunogenic compositions comprising three conjugates

Embodiment

| Ratio option | Ia:Ib:III | Ia:Ib:V | Ia:III:V | Ib:III:V |
|---|---|---|---|---|
| 1 | 1:1:1 | 1:1:1 | 1:1:1 | 1:1:1 |
| 2 | 1:1:2 | 1:1:2 | 1:1:2 | 1:1:2 |
| 3 | 1:1:4 | 1:1:4 | 1:1:4 | 1:1:4 |
| 4 | 1:2:1 | 1:2:1 | 1:2:1 | 1:2:1 |
| 5 | 1:2:2 | 1:2:2 | 1:2:2 | 1:2:2 |
| 6 | 1:2:4 | 1:2:4 | 1:2:4 | 1:2:4 |
| 7 | 1:4:1 | 1:4:1 | 1:4:1 | 1:4:1 |
| 8 | 1:4:2 | 1:4:2 | 1:4:2 | 1:4:2 |
| 9 | 1:4:4 | 1:4:4 | 1:4:4 | 1:4:4 |
| 10 | 2:1:1 | 2:1:1 | 2:1:1 | 2:1:1 |
| 11 | 2:1:2 | 2:1:2 | 2:1:2 | 2:1:2 |
| 12 | 2:1:4 | 2:1:4 | 2:1:4 | 2:1:4 |
| 13 | 2:2:1 | 2:2:1 | 2:2:1 | 2:2:1 |
| 14 | 2:4:1 | 2:4:1 | 2:4:1 | 2:4:1 |
| 15 | 4:1:1 | 4:1:1 | 4:1:1 | 4:1:1 |
| 16 | 4:1:2 | 4:1:2 | 4:1:2 | 4:1:2 |
| 17 | 4:1:4 | 4:1:4 | 4:1:4 | 4:1:4 |
| 18 | 4:2:1 | 4:2:1 | 4:2:1 | 4:2:1 |
| 19 | 4:4:1 | 4:4:1 | 4:4:1 | 4:4:1 |

Of the ratio options described in Table F, the inventors have found that option 1 is effective, particularly when the immunogenic composition comprises: a) a conjugate that is a capsular saccharide from GBS serotype Ia conjugated to a carrier protein; b) a conjugate that is a capsular saccharide from GBS serotype Ib conjugated to a carrier protein; and c) a conjugate that is a capsular saccharide from GBS serotype III conjugated to a carrier protein. This ratio option is therefore preferred for use in the invention, particularly for this embodiment.

TABLE G
Immunogenic compositions comprising four conjugates
Embodiment - Ia, Ib, III & V

| Ratio option | Ia:Ib:III:V |
|---|---|
| 1 | 1:1:1:1 |
| 2 | 1:1:1:2 |
| 3 | 1:1:1:4 |
| 4 | 1:1:2:1 |
| 5 | 1:1:2:2 |
| 6 | 1:1:2:4 |
| 7 | 1:1:4:1 |
| 8 | 1:1:4:2 |
| 9 | 1:1:4:4 |
| 10 | 1:2:1:1 |
| 11 | 1:2:1:2 |
| 12 | 1:2:1:4 |
| 13 | 1:2:2:1 |
| 14 | 1:2:2:2 |
| 15 | 1:2:2:4 |
| 16 | 1:2:4:1 |
| 17 | 1:2:4:2 |
| 18 | 1:2:4:4 |
| 19 | 1:4:1:1 |
| 20 | 1:4:1:2 |
| 21 | 1:4:1:4 |
| 22 | 1:4:2:1 |
| 23 | 1:4:2:2 |
| 24 | 1:4:2:4 |
| 25 | 1:4:4:1 |
| 26 | 1:4:4:2 |
| 27 | 1:4:4:4 |
| 28 | 2:1:1:1 |
| 29 | 2:1:1:2 |
| 30 | 2:1:1:4 |
| 31 | 2:1:2:1 |
| 32 | 2:1:2:2 |
| 33 | 2:1:2:4 |
| 34 | 2:1:4:1 |
| 35 | 2:1:4:2 |
| 36 | 2:1:4:4 |
| 37 | 2:2:1:1 |
| 38 | 2:2:1:2 |
| 39 | 2:2:1:4 |
| 40 | 2:2:2:1 |
| 41 | 2:2:4:1 |
| 42 | 2:4:1:1 |
| 43 | 2:4:1:2 |
| 44 | 2:4:1:4 |
| 45 | 2:4:2:1 |
| 46 | 2:4:4:1 |
| 47 | 4:1:1:1 |
| 48 | 4:1:1:2 |
| 49 | 4:1:1:4 |
| 50 | 4:1:2:1 |
| 51 | 4:1:2:2 |
| 52 | 4:1:2:4 |
| 53 | 4:1:4:1 |
| 54 | 4:1:4:2 |
| 55 | 4:1:4:4 |
| 56 | 4:2:1:1 |
| 57 | 4:2:1:2 |
| 58 | 4:2:1:4 |
| 59 | 4:2:2:1 |
| 60 | 4:2:4:1 |
| 61 | 4:4:1:1 |
| 62 | 4:4:1:2 |
| 63 | 4:4:1:4 |
| 64 | 4:4:2:1 |
| 65 | 4:4:4:1 |

As discussed above, the invention relates in part to immunogenic compositions comprising a conjugate that is a capsular saccharide from GBS serotype V conjugated to a carrier protein. The inventors have found that the immune response to the capsular saccharide from GBS serotype V in these compositions may be diminished if the immunogenic composition comprises one or more further antigen(s). Without wishing to be bound by theory, it is thought that the presence of the further antigen(s) results in “immune interference”, with the response to the capsular saccharide from GBS serotype V being diminished.

The inventors have found that the response to the capsular saccharide from GBS serotype V in these immunogenic compositions may be improved if the composition comprises an adjuvant. This observation is in contrast to the teaching of reference12, which suggests that adjuvants may not improve the immune response to GBS conjugates. Accordingly, in another embodiment the invention provides an immunogenic composition comprising: a) a conjugate that is a capsular saccharide from GBS serotype V conjugated to a carrier protein; b) one or more antigens that do not comprise a capsular saccharide from GBS serotype V; and c) an adjuvant. The antigen(s) of component b) may be conjugate(s) comprising capsular saccharide(s) from other GBS serotype(s). For example, these conjugate(s) may be capsular saccharide(s) from GBS serotype(s) Ia, Ib and/or III conjugated to carrier protein(s). Accordingly, this embodiment of the invention encompasses any of the immunogenic compositions described herein that comprise a conjugate that is a capsular saccharide from GBS serotype V conjugated to a carrier protein and further comprise one or more conjugates that are capsular saccharides from GBS serotypes Ia, Ib and/or III conjugated to carrier proteins, wherein the composition further comprises an adjuvant. Alternatively, the antigen(s) of component b) may be other kind(s) of antigen, e.g. the antigens described under the headings “Combinations of conjugates and other antigens” and “GBS protein antigens” below. Accordingly, this embodiment of the invention also encompasses any of the immunogenic compositions described herein that include a conjugate that is a capsular saccharide from GBS serotype V conjugated to a carrier protein and one or more antigens that do not comprise conjugates comprising capsular saccharide(s) from other GBS serotype(s), wherein the composition further comprises an adjuvant. The inventors have found that the adjuvant in this embodiment of the invention may, for example, be an aluminium salt, as described below. The skilled person would be capable of identifying other adjuvants that may be used in these compositions.

The inventors have also found that the response to the capsular saccharide from GBS serotype V may be improved if the dose of this capsular saccharide is increased. In particular, if the immunogenic composition comprises a conjugate that is a capsular saccharide from a GBS serotype other than type V conjugated to a carrier protein, then the response to the type V capsular saccharide may be improved if the dose of the type V capsular saccharide is greater than the dose of the capsular saccharide from the other GBS serotype. Accordingly, in another embodiment the present invention provides an immunogenic composition comprising: a) a conjugate that is a capsular saccharide from GBS serotype V conjugated to a carrier protein; b) one or more conjugates, each of which is a capsular saccharide from a GBS serotype other than type V conjugated to a carrier protein; wherein the dose of the type V capsular saccharide is greater than the total dose(s) of the capulsular saccharide(s) from the other GBS serotype(s), or is greater than at least one of the doses or the mean dose of the capsular sacchardes from the other GBS serotypes. The dose of the type V capsular saccharide may be 1.1, 2, 3, 4, 5, 6, 7, 8, 9 or 10 times greater. When component b) comprises more than one conjugate, it is typical for the dose of the type V capsular saccharide to be greater than the mean dose of the capsular saccharides from the other GBS serotypes. The conjugate(s) of component b) may be conjugates comprising capsular saccharides from any GBS serotype other than type V. For example, these conjugate(s) may be capsular saccharide(s) from GBS serotype(s) Ia, Ib and/or III conjugated to carrier protein(s). Accordingly, this embodiment of the invention encompasses any of the immunogenic compositions described herein that comprise a conjugate that is a capsular saccharide from GBS serotype V conjugated to a carrier protein and further comprises one or more conjugates that are capsular saccharides from GBS serotypes Ia, Ib and/or III conjugated to carrier proteins; wherein the dose of the type V capsular saccharide is greater than the total dose(s) of the capulsular saccharide(s) from the other GBS serotype(s), or is greater than at least one of the doses or the mean dose of the capsular sacchardes from the other GBS serotypes. The inventors have also found that the immune response to the capsular saccharides from the other GBS serotype(s) in these compositions may be diminished by the greater dose of the type V capsular saccharide. This consequence may be reduced if the composition comprises an adjuvant, as described above. Again, this observation is in contrast to the teaching of reference Error! Bookmark not defined., which suggests that adjuvants may not improve the immune response to GBS conjugates.

Methods of administering the immunogenic compositions of the invention are discussed below. Briefly, the immunogenic compositions of the invention may be administered in single or multiple doses. The inventors have found that the administration of a single dose of the immunogenic compositions of the invention is effective, particularly when the immunogenic composition comprises capsular saccharides from GBS serotypes Ia, Ib and/or III; and more particularly when the immunogenic composition comprises: a) a conjugate that is a capsular saccharide from GBS serotype Ia conjugated to a carrier protein; b) a conjugate that is a capsular saccharide from GBS serotype Ib conjugated to a carrier protein; and c) a conjugate that is a capsular saccharide from GBS serotype III conjugated to a carrier protein. Administration of a single dose is therefore preferred in the invention, particularly for these embodiments.

Alternatively, one unit dose followed by a second unit dose may be effective. Typically, the second (or third, fourth, fifth etc.) unit dose is identical to the first unit dose. The second unit dose may be administered at any suitable time after the first unit dose, in particular after 1, 2 or 3 months. For example, if the immunogenic composition comprises capsular saccharides from GBS serotypes Ia, Ib and/or III, then the second unit dose may be administered 3 months after the first unit dose. In another example, if the immunogenic composition comprises capsular saccharides from GBS serotypes V, then the second unit dose may be administered 1 month after the first unit dose. Typically, the immunogenic compositions of the invention will be administered intramuscularly, e.g. by intramuscular administration to the thigh or the upper arm as described below.

As described below, immunogenic compositions of the invention may include one or more adjuvants. However, the inventors have found that the use of unadjuvanted compositions is effective, particularly when the immunogenic composition comprises capsular saccharides from GBS serotypes Ia, Ib and/or III; and more particularly when the immunogenic composition comprises: a) a conjugate that is a capsular saccharide from GBS serotype Ia conjugated to a carrier protein; b) a conjugate that is a capsular saccharide from GBS serotype Ib conjugated to a carrier protein; and c) a conjugate that is a capsular saccharide from GBS serotype III conjugated to a carrier protein. It may be advantageous to omit adjuvants in order to reduce potential toxicity. Accordingly, immunogenic compositions that do not contain any adjuvant (especially that do not contain any aluminium salt adjuvant) are preferred for use in the invention, particularly for these embodiments.

The Capsular Saccharide

The invention is based on the capsular saccharide of Streptococcus agalactiae. The capsular saccharide is covalently linked to the peptidoglycan backbone of GBS, and is distinct from the group B antigen, which is another saccharide that is attached to the peptidoglycan backbone.

The GBS capsular saccharides are chemically related, but are antigenically very different. All GBS capsular saccharides share the following trisaccharide core:

β-D-GlcpNAc(1→3)β-D-Galp(1→4)β-D-Glcp

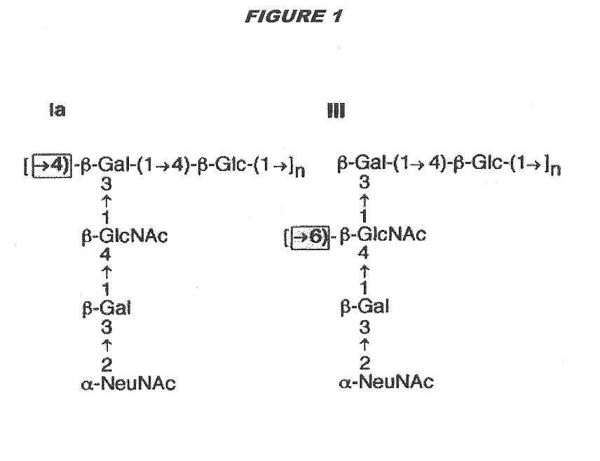

The various GBS serotypes differ by the way in which this core is modified. The difference between serotypes Ia and III, for instance, arises from the use of either the GlcNAc (Ia) or the Gal (III) in this core for linking consecutive trisaccharide cores (FIG. 1). Serotypes Ia and Ib both have a [α-D-NeupNAc(2→3)β-D-Galp-(1→] disaccharide linked to the GlcNAc in the core, but the linkage is either 1→4 (Ia) or 1→3 (Ib).

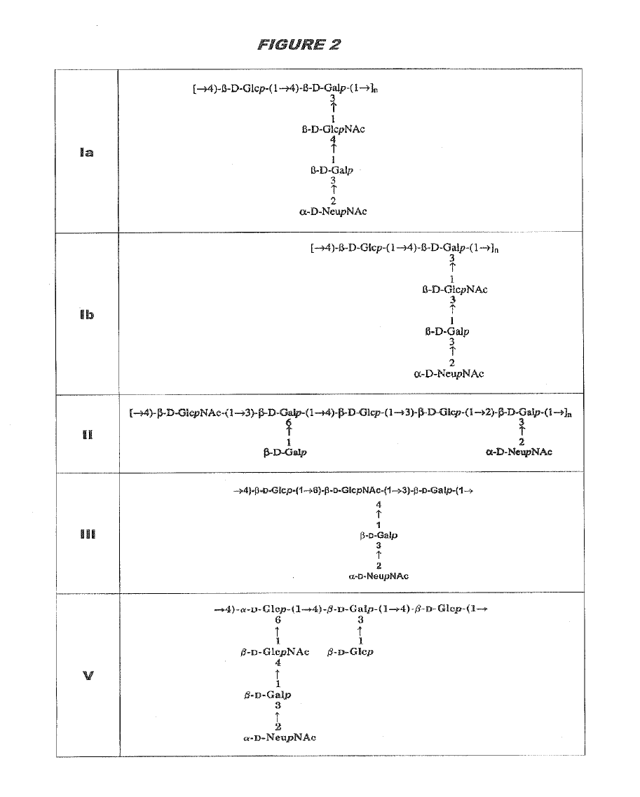

GBS-related disease arises primarily from serotypes Ia, Ib, II, III, IV, V, VI, VII, and VIII, with over 85% being caused by five serotypes: Ia, Ib, III & V. The invention preferably uses a saccharide from one or more of these four serotypes, particularly from one or more of serotypes: Ia, Ib & III. As shown in FIG. 2, the capsular saccharides of each of these four serotypes include: (a) a terminal N-acetyl-neuraminic acid (NeuNAc) residue (commonly referred to as sialic acid), which in all cases is linked 2→3 to a galactose residue; and (b) a N-acetyl-glucosamine residue (GlcNAc) within the trisaccharide core.

All four saccharides include galactose residues within the trisaccharide core, but serotypes Ia, Ib, II & III also contain additional galactose residues in each repeating unit.

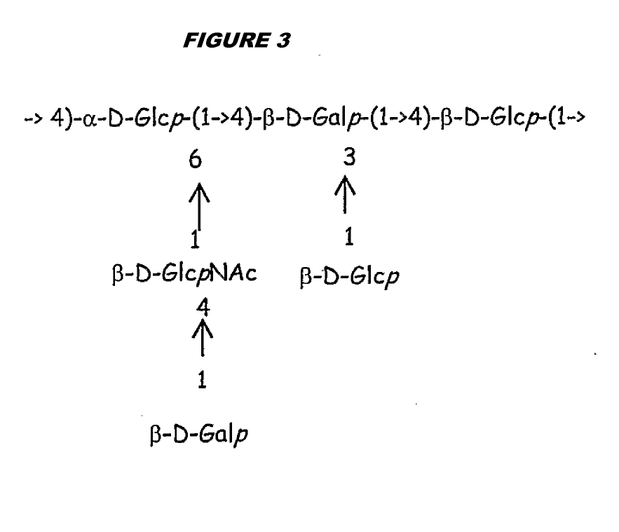

Saccharides used according to the invention may be in their native form, or may have been modified. For example, the saccharide may be shorter than the native capsular saccharide, or may be chemically modified. In particular, the serotype V capsular saccharide used in the invention may be modified as described in refs. 13 and 14. For example, a serotype V capsular saccharide that has been substantially desialylated (FIG. 3) as described in refs. Error! Bookmark not defined and Error! Bookmark not defined. is specifically envisaged for use in the present invention. Desialylated GBS serotype V capsular saccharide may be prepared by treating purified GBS serotype V capsular saccharide under mildly acidic conditions (e.g. 0.1M sulphuric acid at 80° C. for 60 minutes) or by treatment with neuraminidase, as described in reference Error! Bookmark not defined. A preferred method for preparaing desialylated GBS serotype V capsular saccharide is by treating the purified saccharide with 1M acetic acid at 81° C.+/−3C° for 2 h. Thus, the saccharide used according to the invention may be a substantially full-length capsular polysaccharide, as found in nature, or it may be shorter than the natural length. Full-length polysaccharides may be depolymerised to give shorter fragments for use with the invention e.g. by hydrolysis in mild acid, by heating, by sizing chromatography, etc. Chain length has been reported to affect immunogenicity of GBS saccharides in rabbits [Error! Bookmark not defined.]. In particular, the serotype II and/or III capsular saccharides used in the invention may be depolymerised as described in refs. 15 and 16. These documents describe the partial depolymerization of type II and type III capsular saccharides by mild deaminative cleavage to antigenic fragments with reducing-terminal 2,5-anhydro-D-mannose residues. Briefly, the capsular saccharide is dissolved in 0.5 N NaOH and heated at 70° C. for between about 1-4 h. The length of this incubation controls the degree of depolymerisation, which may be determined by standard methods (e.g. by HPLC as described in reference Error! Bookmark not defined.). The sample is chilled in an ice-water bath before glacial acetic acid is added to bring the pH to 4. The partially N-deacylated product is then deaminated by the addition of 5% (wt/vol) NaNO₂ with stirring at 4° C. for 2 h. The free aldehydes of the newly formed 2,5-anhydro-D-mannose residues may be used for conjugation to a carrier protein, as described below.

Depolymerisation of the serotype III capsular saccharide by endo-β-galactosidase has been reported [refs. Error! Bookmark not defined. & Error! Bookmark not defined.-Error! Bookmark not defined.], including using the depolymerised material to form conjugates with a tetanus toxoid carrier. Ozonolysis of capsular polysaccharides from GBS serotypes III and VIII has also been used for depolymerisation [17]. It is preferred to use saccharides with MW>30 kDa, and substantially full-length capsular polysaccharides can be used. For serotype Ia, it is preferred to use polysaccharides with a MW in the range of 150-300 kDa, particularly 175-275 kDa. Typically, a serotype Ia saccharide with MW about 200 kDa or about 260 kDa is used. For serotype Ib, it is preferred to use polysaccharides with a MW in the range of 150-300 kDa, particularly 175-250 kDa. Typically, a serotype Ib saccharide with MW about 200 kDa or about 230 kDa is used. For serotype III, it is preferred to use polysaccharides with a MW in the range of 50-200 kDa, particularly 80-150 kDa. Typically, a serotype III saccharide with MW about 100 kDa or about 140 kDa is used. For serotype V, it is also preferred to use polysaccharides with a MW up to ˜50 kDa. Typically, a serotype V saccharide with MW about 100 kDa is used. These molecular masses can be measured by gel filtration relative to dextran standards, such as those available from Polymer Standard Service [18].

The saccharide may be chemically modified relative to the capsular saccharide as found in nature. For example, the saccharide may be de-O-acetylated (partially or fully), de-N-acetylated (partially or fully), N-propionated (partially or fully), etc. De-acetylation may occur before, during or after conjugation, but preferably occurs before conjugation. Depending on the particular saccharide, de-acetylation may or may not affect immunogenicity. The relevance of O-acetylation on GBS saccharides in various serotypes is discussed in reference 19, and in some embodiments O-acetylation of sialic acid residues at positions 7, 8 and/or 9 is retained before, during and after conjugation e.g. by protection/de-protection, by re-acetylation, etc. However, typically the GBS saccharide used in the present invention has substantially no O-acetylation of sialic acid residues at positions 7, 8 and/or 9. In particular, when the GBS saccharide has been purified by base extraction as described below, then O-acetylation is typically lost (ref. Error! Bookmark not defined.). The effect of de-acetylation etc. can be assessed by routine assays.

Capsular saccharides can be purified by known techniques, as described in the references herein such as refs. Error! Bookmark not defined. and 20. A typical process involves base extraction, centrifugation, filtration, RNase/DNase treatment, protease treatment, concentration, size exclusion chromatography, ultrafiltration, anion exchange chromatography, and further ultrafiltration. Treatment of GBS cells with the enzyme mutanolysin, which cleaves the bacterial cell wall to free the cell wall components, is also useful.

As an alternative, the purification process described in reference 21 can be used. This involves base extraction, ethanol/CaCl₂ treatment, CTAB precipitation, and re-solubilisation. A further alternative process is described in reference 22.

The invention is not limited to saccharides purified from natural sources, however, and the saccharides may be obtained by other methods, such as total or partial synthesis.

Conjugation

The invention involves conjugates that are capsular saccharides from GBS serotypes Ia, Ib, III or V conjugated to a carrier protein. In general, covalent conjugation of saccharides to carriers enhances the immunogenicity of saccharides as it converts them from T-independent antigens to T-dependent antigens, thus allowing priming for immunological memory. Conjugation is particularly useful for paediatric vaccines [e.g. ref 23] and is a well known technique [e.g. reviewed in refs. 24 to ]. Thus the processes of the invention may include the further step of conjugating the purified saccharide to a carrier molecule.

Conjugation of GBS saccharides has been widely reported e.g. see references Error! Bookmark not defined. to. The typical prior art process for GBS saccharide conjugation typically involves reductive amination of a purified saccharide to a carrier protein such as tetanus toxoid (TT) or CRM197 [2]. The reductive amination involves an amine group on the side chain of an amino acid in the carrier and an aldehyde group in the saccharide. As GBS capsular saccharides do not include an aldehyde group in their natural form then this is typically generated before conjugation by oxidation (e.g. periodate oxidation) of a portion (e.g. between 5 and 40%, particularly between 10 and 30%, preferably about 20%) of the saccharide's sialic acid residues [2,33]. Conjugate vaccines prepared in this manner have been shown to be safe and immunogenic in humans for each of GBS serotypes Ia, Ib, II, III, and V [10]. Typically, all of the conjugates in the immunogenic compositions of the present invention have been prepared in this manner. However, when the invention uses a serotype V capsular saccharide that is desialylated, then an aldehyde group may be generated in this saccharide before conjugation by oxidation (e.g. periodate oxidation) of a portion (e.g. between 5 and 40%, particularly between 10 and 30%, preferably about 20%) of the saccharide's galactose residues [14]. An alternative conjugation process involves the use of —NH₂ groups in the saccharide (either from de-N-acetylation, or after introduction of amines) in conjunction with bifunctional linkers, as described in ref. 34. In some embodiments, one or more of the conjugates in the immunogenic compositions of the present invention have been prepared in this manner. A further alternative process is described in refs. 15 and 16. In this process, the free aldehydes groups of terminal 2,5-anhydro-D-mannose residues from depolymerization of type II or type III capsular saccharides by mild deaminative cleavage are used for conjugation by reductive amination. In some embodiments, one or more of the conjugates in the immunogenic compositions of the present invention have been prepared in this manner

The invention involves the use of carrier molecules, which are typically proteins. Useful carrier proteins include bacterial toxins or toxoids, such as diphtheria toxoid or tetanus toxoid. Fragments of toxins or toxoids can also be used e.g. fragment C of tetanus toxoid [35]. The CRM197 mutant of diphtheria toxin [36-38] is particularly useful with the invention. Other suitable carrier proteins include the N. meningitidis outer membrane protein [39], synthetic peptides [40,41], heat shock proteins [42,43], pertussis proteins [44,45], cytokines [46], lymphokines [46], hormones [46], growth factors [46], human serum albumin (preferably recombinant), artificial proteins comprising multiple human CD4⁺ T cell epitopes from various pathogen-derived antigens [47] such as N19 [48], protein D from H. influenzae [49,50], pneumococcal surface protein PspA [51], pneumolysin [52], iron-uptake proteins [53], toxin A or B from C.difficile [54], recombinant Pseudomonas aeruginosa exoprotein A (rEPA) [55], a GBS protein (see below; particularly GBS67) [206], etc.

Attachment to the carrier is preferably via a —NH₂ group e.g. in the side chain of a lysine residue in a carrier protein, or of an arginine residue, or at the N-terminus. Attachment may also be via a —SH group e.g. in the side chain of a cysteine residue.

It is possible to use more than one carrier protein e.g. to reduce the risk of carrier suppression. Thus different carrier proteins can be used for different GBS serotypes e.g. serotype Ia saccharides might be conjugated to CRM197 while serotype Ib saccharides might be conjugated to tetanus toxoid. It is also possible to use more than one carrier protein for a particular saccharide antigen e.g. serotype III saccharides might be in two groups, with some conjugated to CRM197 and others conjugated to tetanus toxoid. In general, however, it is preferred to use the same carrier protein for all saccharides.

A single carrier protein might carry more than one saccharide antigen [56,57]. For example, a single carrier protein might have conjugated to it saccharides from serotypes Ia and Ib. To achieve this goal, different saccharides can be mixed prior to the conjugation reaction. In general, however, it is preferred to have separate conjugates for each serogroup, with the different saccharides being mixed after conjugation. The separate conjugates may be based on the same carrier.

Conjugates with a saccharide:protein ratio (w/w) of between 1:5 (i.e. excess protein) and 5:1 (i.e. excess saccharide) are typically used, in particular ratios between 1:5 and 2:1. When the invention uses a conjugate that is a capsular saccharide from GBS serotype Ia conjugated to a carrier protein, then the saccharide:protein ratio (w/w) is typically between about 1:1 to 1:2, particularly about 1:1.3. Similarly, when the invention uses a conjugate that is a capsular saccharide from GBS serotype Ib conjugated to a carrier protein, then the ratio is typically between about 1:1 to 1:2, particularly about 1:1.3. When the invention uses a conjugate that is a capsular saccharide from GBS serotype III conjugated to a carrier protein, then the saccharide:protein ratio (w/w) is typically between about 3:1 to 1:1, particularly about 2:1. However, GBS serotype III conjugated to a carrier protein with a saccharide:protein ratio (w/w) of about 1:1 to 1:5, particularly about 1:3.3, may also be used. Finally, when the invention uses a conjugate that is a capsular saccharide from GBS serotype V conjugated to a carrier protein, then the ratio is typically between about 2:1 to 1:1, particularly about 1.1:1. Thus a weight excess of saccharide is typical, particularly with longer saccharide chains.

Compositions may include a small amount of free carrier [58]. When a given carrier protein is present in both free and conjugated form in a composition of the invention, the unconjugated form is preferably no more than 5% of the total amount of the carrier protein in the composition as a whole, and more preferably present at less than 2% by weight.

After conjugation, free and conjugated saccharides can be separated. There are many suitable methods, including hydrophobic chromatography, tangential ultrafiltration, diafiltration etc. [see also refs. 59 & 60, etc.]. A preferred method is described in reference 61.

Where the composition of the invention includes a depolymerised oligosaccharide, it is preferred that depolymerisation precedes conjugation.

Combinations of Conjugates and Other Antigens

The immunogenic compositions of the invention may comprise one or more further antigens.

The further antigen(s) may comprise further GBS conjugates. The different GBS conjugates may include different types of conjugate from the same GBS serotype and/or conjugates from different GBS serotypes. The composition will typically be produced by preparing separate conjugates (e.g. a different conjugate for each serotype) and then combining the conjugates.

The further antigen(s) may comprise GBS amino acid sequences, as set out below.

The further antigen(s) may comprise antigens from non-GBS pathogens. Thus the compositions of the invention may further comprise one or more non-GBS antigens, including additional bacterial, viral or parasitic antigens. These may be selected from the following:
a protein antigen from N.meningitidis serogroup B, such as those in refs. 62 to, with protein ‘287’ (see below) and derivatives (e.g. ‘ΔG287’) being particularly preferred.
an outer-membrane vesicle (OMV) preparation from N.meningitidis serogroup B, such as those disclosed in refs. 69, 70, 71, 72 etc.
a saccharide antigen from N.meningitidis serogroup A, C, W135 and/or Y, such as the oligosaccharide disclosed in ref 73 from serogroup C or the oligosaccharides of ref. 74.
a saccharide antigen from Streptococcus pneumoniae [e.g. refs. 75-77; chapters 22 & 23 of ref. 84].
an antigen from hepatitis A virus, such as inactivated virus [e.g. 78, 79; chapter 15 of ref. 84].
an antigen from hepatitis B virus, such as the surface and/or core antigens [e.g. 79,80; chapter16 of ref. Error! Bookmark not defined.].
an antigen from hepatitis C virus [e.g. 81].
an antigen from Bordetella pertussis, such as pertussis holotoxin (PT) and filamentous haemagglutinin (FHA) from B.pertussis, optionally also in combination with pertactin and/or agglutinogens 2 and 3 [e.g. refs. 82 & 83; chapter 21 of ref. 84].
a diphtheria antigen, such as a diphtheria toxoid [e.g. chapter 13 of ref. 84].
a tetanus antigen, such as a tetanus toxoid [e.g. chapter 27 of ref. 84].
a saccharide antigen from Haemophilus influenzae B [e.g. chapter 14 of ref 84]
an antigen from N.gonorrhoeae [e.g. 62, 63, 64].
an antigen from Chlamydia pneumoniae [e.g. 85, 86, 87, 88, 89, 90, 91].
an antigen from Chlamydia trachomatis [e.g. 92].
an antigen from Porphyromonas gingivalis [e.g. 93].
polio antigen(s) [e.g. 94, 95; chapter 24 of ref. Error! Bookmark not defined.] such as IPV.
rabies antigen(s) [e.g. 96] such as lyophilised inactivated virus [e.g. 97 RABAVERT™].
measles, mumps and/or rubella antigens [e.g. chapters 19, 20 and 26 of ref.84].
influenza antigen(s) [e.g. chapters 17 & 18 of ref. 84], such as the haemagglutinin and/or neuraminidase surface proteins.
an antigen from Moraxella catarrhalis [e.g. 98].
an antigen from Streptococcus pyogenes (group A streptococcus) [e.g. 99, 100, 101].
an antigen from Staphylococcus aureus [e.g 102].
Where a saccharide or carbohydrate antigen is used, it is preferably conjugated to a carrier in order to enhance immunogenicity. Conjugation of H. influenzae B, meningococcal and pneumococcal saccharide antigens is well known.

Toxic protein antigens may be detoxified where necessary (e.g. detoxification of pertussis toxin by chemical and/or genetic means [83]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens may be adsorbed to an aluminium salt. Where there is more than one conjugate in a composition, not all conjugates need to be adsorbed.

One type of preferred composition includes further antigens from sexually-transmitted pathogens, such as: herpes virus; N.gonorrhoeae; C. trachomatis; etc. Another type of preferred composition includes further antigens that affect the elderly and/or the immunocompromised, and so the GBS antigens of the invention can be combined with one or more antigens from the following non-GBS pathogens: influenza virus, Enterococcus faecalis, Staphylococcus aureus, Staphylococcus epidermis, Pseudomonas aeruginosa, Legionella pneumophila, Listeria monocytogenes, Neisseria meningitidis, and parainfluenza virus.

Antigens in the composition will typically be present at a concentration of at least 1 μg/ml each. In general, the concentration of any given antigen will be sufficient to elicit an immune response against that antigen.

As an alternative to using proteins antigens in the composition of the invention, nucleic acid encoding the antigen may be used [e.g. refs. 103 to 111]. Protein components of the compositions of the invention may thus be replaced by nucleic acid (preferably DNA e.g. in the form of a plasmid) that encodes the protein.

In practical terms, there may be an upper limit to the number of antigens included in compositions of the invention. The number of antigens (including GBS antigens) in a composition of the invention may be less than 20, less than 19, less than 18, less than 17, less than 16, less than 15, less than 14, less than 13, less than 12, less than 11, less than 10, less than 9, less than 8, less than 7, less than 6, less than 5, less than 4, less than 3, or less than 2. The number of GBS antigens in a composition of the invention may be less than 6, less than 5, less than 4, less than 3, or less than 2.

Pharmaceutical Methods and Uses

The immunogenic compositions of the invention may further comprise a pharmaceutically acceptable carrier. Typical ‘pharmaceutically acceptable carriers’ include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose [112], trehalose [113], lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in reference 114.

Compositions of the invention may be in aqueous form (i.e. solutions or suspensions) or in a dried form (e.g. lyophilised). If a dried vaccine is used then it will be reconstituted into a liquid medium prior to injection. Lyophilisation of conjugate vaccines is known in the art e.g. the MENJUGATE™ product is presented in lyophilised form. When the immunogenic compositions of the invention include conjugates comprising capsular saccharides from more than one GBS serotypes, it is typical for the conjugates to be prepared separately, mixed and then lyophilised. In this way, lyophilised compositions comprising two, three or four etc. conjugates as described herein may be prepared. To stabilise conjugates during lyophilisation, it may be preferred to include a sugar alcohol (e.g. mannitol) and/or a disaccharide (e.g. sucrose or trehalose) e.g. at between 1 mg/ml and 30 mg/ml (e.g. about 25 mg/ml) in the composition. The use of sucrose has been recommended as a stabiliser for GBS conjugate vaccines (ref 115). However, it is typical for the stabiliser of the present invention to be mannitol. When the dried vaccine is reconstituted into a liquid medium prior to injection, the concentration of residual mannitol will typically be about 2-20 mg/ml, e.g. 3.75 mg/ml, 7.5 mg/ml or 15 mg/ml. The use of mannitol is advantageous because mannitol is chemically distinct from the monosaccharide subunits of the GBS capsular saccharides. This means that detection of the capsular saccharides, e.g. for quality control analysis, can be based on the presence of the subunits of the saccharides without interference from the mannitol. In contrast, a stabiliser like sucrose contains glucose, which may interfere with the detection of glucose subunits in the saccharides.

Compositions may be presented in vials, or they may be presented in ready-filled syringes. The syringes may be supplied with or without needles. A syringe will include a single dose of the composition, whereas a vial may include a single dose or multiple doses.

Aqueous compositions of the invention are also suitable for reconstituting other vaccines from a lyophilised form. Where a composition of the invention is to be used for such extemporaneous reconstitution, the invention provides a kit, which may comprise two vials, or may comprise one ready-filled syringe and one vial, with the contents of the syringe being used to reactivate the contents of the vial prior to injection.

Compositions of the invention may be packaged in unit dose form or in multiple dose form. For multiple dose forms, vials are preferred to pre-filled syringes. Effective dosage volumes can be routinely established, but a typical human dose of the composition has a volume of 0.5 ml e.g. for for intramuscular injection.

The pH of the composition is preferably between 6 and 8, preferably about 7. Stable pH may be maintained by the use of a buffer. The immunogenic compositions of the invention typically comprise a potassium dihydrogen phosphate buffer. The potassium dihydrogen phosphate buffer may comprise about 1-10 mM potassium dihydrogen phosphate, e.g. 1.25 mM, 2.5 mM or 5.0 mM. If a composition comprises an aluminium hydroxide salt, it is preferred to use a histidine buffer [116]. The composition may be sterile and/or pyrogen-free. Compositions of the invention may be isotonic with respect to humans.

Compositions of the invention are immunogenic, and are more preferably vaccine compositions. Vaccines according to the invention may either be prophylactic (i.e. to prevent infection) or therapeutic (i.e. to treat infection), but will typically be prophylactic Immunogenic compositions used as vaccines comprise an immunologically effective amount of antigen(s), as well as any other components, as needed. By ‘immunologically effective amount’, it is meant that the admin-istration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

Within each dose, the quantity of an individual saccharide antigen will generally be between 0.1-50 μg (measured as mass of saccharide), particularly between 1-50 μg or 0.5-25 μg, more particularly 2.5-7.5 g, e.g. about 1 μg, about 2.5 μg, about 5 μg, about 10 μg, about 15 μg, about 20 μg or about 25 μg. Within each dose, the total quantity of GBS capsular saccharides will generally be ≦70 μg (measured as mass of saccharide), e.g. ≦60 μg. In particular, the total quantity may be ≦40 μg (e.g. ≦30 μg) or ≦20 μg (e.g. ≦15 μg). The inventors have found that these total quantities are effective, particularly when the immunogenic composition comprises: a) a conjugate that is a capsular saccharide from GBS serotype Ia conjugated to a carrier protein; b) a conjugate that is a capsular saccharide from GBS serotype Ib conjugated to a carrier protein; and c) a conjugate that is a capsular saccharide from GBS serotype III conjugated to a carrier protein. These total quantities are therefore preferred for use in the invention, particularly for this embodiment. It may be advantageous to minimise the total quantity of capsular saccharide(s) per unit dose in order to reduce potential toxicity. Accordingly, a total quantity of ≦20 μg is preferred, e.g. ≦15 μg, ≦7.5 μg or ≦1.5 μg.

GBS affects various areas of the body and so the compositions of the invention may be prepared in various forms. For example, the compositions may be prepared as injectables, either as liquid solutions or suspensions. The composition may be prepared for pulmonary administration e.g. as an inhaler, using a fine powder or a spray. The composition may be prepared as a suppository or pessary. The composition may be prepared for nasal, aural or ocular administration e.g. as spray, drops, gel or powder [e.g. refs 117 & 118]. Success with nasal administration of pneumococcal saccharides [119,120], Hib saccharides [121], MenC saccharides [122], and mixtures of Hib and MenC saccharide conjugates [123] has been reported.

Compositions of the invention may include an antimicrobial, particularly when packaged in multiple dose format.

Compositions of the invention may comprise detergent e.g. a TWEEN™ (polysorbate), such as TWEEN™80. Detergents are generally present at low levels e.g. <0.01%.

Compositions of the invention may include sodium salts (e.g. sodium chloride) to give tonicity. A concentration of 10±2 mg/ml NaCl is typical. In some embodiments, a concentration of 4-10 mg/ml NaCl may be used, e.g. 9.0, 7.0, 6.75 or 4.5 mg/ml.

Compositions of the invention will generally include a buffer. A phosphate buffer is typical.

Compositions of the invention may be administered in conjunction with other immunoregulatory agents. In particular, compositions may include one or more adjuvants. Such adjuvants include, but are not limited to:

A. Mineral-Containing Compositions

Mineral containing compositions suitable for use as adjuvants in the invention include mineral salts, such as aluminium salts and calcium salts (or mixtures thereof). Calcium salts include calcium phosphate (e.g. the “CAP” particles disclosed in ref 124). Aluminum salts include hydroxides, phosphates, sulfates, etc., with the salts taking any suitable form (e.g. gel, crystalline, amorphous, etc.). Adsorption to these salts is preferred. The mineral-containing compositions may also be formulated as a particle of metal salt [125].

The adjuvants known as aluminum hydroxide and aluminum phosphate may be used. These names are conventional, but are used for convenience only, as neither is a precise description of the actual chemical compound which is present (e.g. see chapter 9 of reference 126). The invention can use any of the “hydroxide” or “phosphate” adjuvants that are in general use as adjuvants. The adjuvants known as “aluminium hydroxide” are typically aluminium oxyhydroxide salts, which are usually at least partially crystalline. The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt.

A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

Aluminium phosphate adjuvants generally have a PO₄/Al molar ratio between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, preferably between 5 and 15 mM, and more preferably about 10 mM. The suspensions may also comprise sodium chloride.

The invention can use a mixture of both an aluminium hydroxide and an aluminium phosphate. In this case there may be more aluminium phosphate than hydroxide e.g. a weight ratio of at least 2:1 e.g.≧5:1, ≧6:1, ≧7:1, ≧8:1, ≧9:1, etc.

The concentration of Al⁺⁻⁺ in a composition for administration to a patient is preferably less than 10 mg/ml e.g. ≦5 mg/ml, ≦4 mg/ml, ≦3 mg/ml, ≦2 mg/ml, ≦1 mg/ml, etc. A preferred range is between 0.3 and 1 mg/ml. A maximum of 0.85 mg/dose is preferred.

A typical adjuvant aluminium phosphate adjuvant is amorphous aluminium hydroxyphosphate with PO₄/Al molar ratio between 0.84 and 0.92, included at 0.6 mg Al³⁺/ml. Adsorption with a low dose of aluminium phosphate may be used e.g. between 50 and 100 μg Al³⁺ per conjugate per dose.

B. Oil Emulsions

Oil emulsion compositions suitable for use as adjuvants in the invention include squalene-water emulsions, such as MF59 (5% Squalene, 0.5% TWEEN™80, and 0.5% SPAN™85, formulated into submicron particles using a microfluidizer) [Chapter 10 of ref 126; see also refs. [127-129]. MF59 is used as the adjuvant in the FLUAD™ influenza virus trivalent subunit vaccine.

Particularly preferred adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v TWEEN™80 (polyoxyethylenesorbitan monooleate), and/or 0.25-1.0% SPAN™85 (sorbitan trioleate), and, optionally, N-acetylmuramyl-L-alanyl-D-isoglutaminyl -L-alanine-2-(1′-2′-dipalmitoyl-sn-glycero-3-hydroxyphosphophoryloxy)-ethylamine (MTP-PE). Submicron oil-in-water emulsions, methods of making the same and immunostimulating agents, such as muramyl peptides, for use in the compositions, are described in detail in references & 127 & 130-131.

Complete Freund's adjuvant (CFA) and incomplete Freund's adjuvant (IFA) may also be used as adjuvants in the invention.

C. Saponin Formulations [chapter 22 of ref. Error! Bookmark not defined.]

Saponin formulations may also be used as adjuvants in the invention. Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref 132. Saponin formulations may also comprise a sterol, such as cholesterol [133].

Combinations of saponins and cholesterols can be used to form unique particles called immunostimulating complexes (ISCOMs) [chapter 23 of ref 126]. ISCOMs typically also include a phospholipid such as phosphatidylethanolamine or phosphatidylcholine. Any known saponin can be used in ISCOMs. Preferably, the ISCOM includes one or more of QuilA, QHA and QHC. ISCOMs are further described in refs. 133-135. Optionally, the ISCOMS may be devoid of additional detergent(s) [136].

A review of the development of saponin based adjuvants can be found in refs. 137 & 138.

D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, QB-phage (such as coat proteins), GA-phage, fr-phage, AP205 phage, and Ty (such as retrotransposon Ty protein pl). VLPs are discussed further in refs. 139-144. Virosomes are discussed further in, for example, ref 145.

E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref 146. Such “small particles” of 3dMPL are small enough to be sterile filtered through a 0.22 μm membrane [146]. Other non-toxic LPS derivatives include monophosphoryl lipid A mimics, such as aminoalkyl glucosaminide phosphate derivatives e.g. RC-529 [147,148].

Lipid A derivatives include derivatives of lipid A from Escherichia coli such as OM-174. OM-174 is described for example in refs. 149 & 150.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 151, 152 and 153 disclose possible analog substitutions e.g. replacement of guanosine with 2′-deoxy-7-deazaguanosine. The adjuvant effect of CpG oligonucleotides is further discussed in refs. 154-159.

The CpG sequence may be directed to TLR9, such as the motif GTCGTT or TTCGTT [160]. The CpG sequence may be specific for inducing a Thl immune response, such as a CpG-A ODN, or it may be more specific for inducing a B cell response, such a CpG-B ODN. CpG-A and CpG-B ODNs are discussed in refs. 161-163. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 160 & 164-166.

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref 167 and as parenteral adjuvants in ref 168. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivaties thereof, particularly LT-K63 and LT-R72, as adjuvants can be found in refs. 169-176. Numerical reference for amino acid substitutions is preferably based on the alignments of the A and B subunits of ADP-ribosylating toxins set forth in ref 177, specifically incorporated herein by reference in its entirety.

F. Human Immunomodulators

Human immunomodulators suitable for use as adjuvants in the invention include cytokines, such as interleukins (e.g. IL-1, IL-2, IL-4, IL-5, IL-6, IL-7, IL-12 [178], etc.) [179], interferons (e.g. interferon-γ), macrophage colony stimulating factor, and tumor necrosis factor.

G. Bioadhesives and Mucoadhesives

Bioadhesives and mucoadhesives may also be used as adjuvants in the invention. Suitable bioadhesives include esterified hyaluronic acid microspheres [180] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [181].

H. Microparticles

Microparticles may also be used as adjuvants in the invention. Microparticles (i.e. a particle of ˜100 nm to ˜150 μm in diameter, more preferably ˜200nm to ˜30 μm in diameter, and most preferably ˜500 nm to ˜10 μm in diameter) formed from materials that are biodegradable and non-toxic (e.g. a poly(a-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

I. Liposomes (Chapters 13 & 14 of ref 126)

Examples of liposome formulations suitable for use as adjuvants are described in refs. 182-184.

J. Polyoxyethylene Ether and Polyoxyethylene Ester Formulations

Adjuvants suitable for use in the invention include polyoxyethylene ethers and polyoxyethylene esters [185]. Such formulations further include polyoxyethylene sorbitan ester surfactants in combination with an octoxynol [186] as well as polyoxyethylene alkyl ethers or ester surfactants in combination with at least one additional non-ionic surfactant such as an octoxynol [187]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

K. Polyphosphazene (PCPP)

PCPP formulations are described, for example, in refs.188 and 189.

L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

M. Imidazoquinolone Compounds.

Examples of imidazoquinolone compounds suitable for use adjuvants in the invention include Imiquamod and its homologues (e.g. “Resiquimod 3M”), described further in refs. 190 and 191.

N. Thiosemicarbazone Compounds.

Examples of thiosemicarbazone compounds, as well as methods of formulating, manufacturing, and screening for compounds all suitable for use as adjuvants in the invention include those described in ref 192. The thiosemicarbazones are particularly effective in the stimulation of human peripheral blood mononuclear cells for the production of cytokines, such as TNF-α.

O. Tryptanthrin Compounds.

Examples of tryptanthrin compounds, as well as methods of formulating, manufacturing, and screening for compounds all suitable for use as adjuvants in the invention include those described in ref 193. The tryptanthrin compounds are particularly effective in the stimulation of human peripheral blood mononuclear cells for the production of cytokines, such as TNF-α.

The invention may also comprise combinations of aspects of one or more of the adjuvants identified above. For example, the following combinations may be used as adjuvant compositions in the invention: (1) a saponin and an oil-in-water emulsion [194]; (2) a saponin (e.g. QS21)+a non-toxic LPS derivative (e.g. 3dMPL) [195]; (3) a saponin (e.g. QS21)+a non-toxic LPS derivative (e.g. 3dMPL)+a cholesterol; (4) a saponin (e.g. QS21)+3dMPL +IL-12 (optionally+a sterol) [196]; (5) combinations of 3dMPL with, for example, QS21 and/or oil-in-water emulsions [197]; (6) SAF, containing 10% squalane, 0.4% TWEEN™80, 5% pluronic-block polymer L121, and thr-MDP, either microfluidized into a submicron emulsion or vortexed to generate a larger particle size emulsion. (7) RIBI™ adjuvant system (RAS), (Ribi Immunochem) containing 2% squalene, 0.2% TWEEN™ 80, and one or more bacterial cell wall components from the group consisting of monophosphorylipid A (MPL), trehalose dimycolate (TDM), and cell wall skeleton (CWS), preferably MPL+CWS (DETOX™); and (8) one or more mineral salts (such as an aluminum salt)+a non-toxic derivative of LPS (such as 3dMPL).

Other substances that act as immunostimulating agents are disclosed in chapter 7 of ref. 126.

The use of aluminium salt adjuvants is particularly preferred, and antigens are generally adsorbed to such salts. It is possible in compositions of the invention to adsorb some antigens to an aluminium hydroxide but to have other antigens in association with an aluminium phosphate. In general, however, it is preferred to use only a single salt e.g. a hydroxide or a phosphate, but not both. Not all conjugates need to be adsorbed i.e. some or all can be free in solution.

Methods of Treatment

The invention also provides a method for raising an immune response in a mammal, comprising administering a pharmaceutical composition of the invention to the mammal. The immune response is preferably protective and preferably involves antibodies. The method may raise a booster response.

The mammal is preferably a human Where the vaccine is for prophylactic use, the human is preferably a child (e.g. a toddler or infant) or a teenager; where the vaccine is for therapeutic use, the human is preferably an adult. A vaccine intended for children may also be administered to adults e.g. to assess safety, dosage, immunogenicity, etc. A preferred class of humans for treatment are females of child-bearing age (e.g. teenagers and above). Another preferred class is pregnant females. Elderly patients (e.g. those above 50, 60, 70, 80 or 90 etc. years of age, particularly over 65 years of age), especially those living in nursing homes where the risk of GBS infection may be increased ([198]), are another preferred class of humans for treatment. In some embodiments, the human has an undetectable level of antibodies against capsular saccharide from GBS serotype Ia prior to administration of the pharmaceutical composition. In other embodiments, the human has an undetectable level of antibodies against capsular saccharide from GBS serotype Ib prior to administration of the pharmaceutical composition. In other embodiments, the human has an undetectable level of antibodies against capsular saccharide from GBS serotype III prior to administration of the pharmaceutical composition. In particular, the human may have an undetectable level of antibodies against capsular saccharide from GBS serotype Ia and an undetectable level of antibodies against capsular saccharide from GBS serotype Ib prior to administration of the pharmaceutical composition. Alternatively or in addition, the human may have an undetectable level of antibodies against capsular saccharide from GBS serotype III prior to administration of the pharmaceutical composition. The level(s) of antibodies against the capsular saccharide(s) may be determined using the ELISA described in Human study (1) below. The level(s) of antibodies may be as of one month prior to administration, particularly within one month prior to administration (e.g. within two weeks, within one week or on the day of administration). Women with these undetectable level(s) of antibodies against the capsular saccharide(s) may have higher rates of GBS infection in their newborns. This is because higher levels of maternal antibodies against GBS capsular saccharides are correleated with reduced risk of disease in newborns [refs. 199 and 200]. Accordingly, administration to these women is specifically envisaged in the present invention.

In some embodiments, the patient has been pre-immunised with a diphtheria toxoid or derivative thereof, e.g. as described below with respect to the second aspect of the invention in the section The pre-immunised patient. In these embodiments, it is preferred for at least one conjugate in the immunogenic composition to be a capsular saccharide from GBS conjugated to a diphtheria toxoid or derivative thereof. The inventors have found that the immune response to the capsular saccharide may be improved by presenting the saccharide on a diphtheria toxoid or derivative thereof, when the patient has been pre-immunised with a diphtheria toxoid or derivative thereof. The capsular saccharide conjugated to the diphtheria toxoid or derivative thereof in the composition may for example be from GBS serotype la, Ib or III. In particular, the capsular saccharide may be from GBS serotype III (as exemplified below). In these embodiments, it is typical for all of the capsular saccharides from GBS in the composition to be conjugated to a diphtheria toxoid or derivative thereof. Where the carrier or pre-immunisation antigen is a derivative of a diphtheria toxoid then that derivative preferably remains immunologically cross-reactive with Dt, and is preferably CRM197.

In other embodiments, the patient has been pre-immunised with a tetanus toxoid or derivative thereof, e.g. as described below with respect to the second aspect of the invention in the sections The pre-immunised patient and Tetanus toxoid carriers. In these embodiments, it is preferred for at least one conjugate in the immunogenic composition to be a capsular saccharide from GBS conjugated to a tetanus toxoid or derivative thereof. The immune response to the capsular saccharide may be improved by presenting the saccharide on a tetanus toxoid or derivative thereof, when the patient has been pre-immunised with a tetanus toxoid or derivative thereof. The capsular saccharide conjugated to the tetanus toxoid or derivative thereof in the composition may for example be from GBS serotype la, Ib or III. In particular, the capsular saccharide may be from GBS serotype III. In these embodiments, it is typical for all of the capsular saccharides from GBS in the composition to be conjugated to a tetanus toxoid or derivative thereof.

The invention also provides a composition of the invention for use as a medicament. The medicament is preferably able to raise an immune response in a mammal (i.e. it is an immunogenic composition) and is more preferably a vaccine.

The invention also provides the use of a composition of the invention in the manufacture of a medicament for raising an immune response in a mammal.

These uses and methods are preferably for the prevention and/or treatment of a disease caused by S. agalactiae e.g. neonatal sepsis or bacteremia, neonatal pneumonia, neonatal meningitis, endometritis, osteomyelitis, septic arthritis, etc.

The subject in which disease is prevented may not be the same as the subject that receives the conjugate of the invention. For instance, a conjugate may be administered to a female (before or during pregnancy) in order to protect offspring (so-called ‘maternal immunisation’ [201-203]).

One way of checking efficacy of therapeutic treatment involves monitoring GBS infection after administration of the composition of the invention. One way of checking efficacy of prophylactic treatment involves monitoring immune responses against the GBS antigens after administration of the composition.

Preferred compositions of the invention can confer an antibody titre in a patient that is superior to the criterion for seroprotection for each antigenic component for an acceptable percentage of human subjects. Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is 0.5 ml.

The invention may be used to elicit systemic and/or mucosal immunity.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

GBS Protein Antigens

As mentioned above, GBS proteins can be included in compositions of the invention. These may be used as carrier proteins for conjugates of the invention, carrier proteins for other conjugates, or as unconjugated protein antigens.

GBS protein antigens for use with the invention include those disclosed in references 99 and 204-206. Two preferred GBS protein antigens for use with the invention are known as: GBS67; and GBS80 [see ref 99]. A further preferred GBS protein antigen for use with the invention is known as Spb1 [see ref 270]. Further details of these three antigens are given below.

The full-length sequences for these three GBS proteins are SEQ ID NOs 1 to 3 herein. Compositions of the invention may thus include (a) a polypeptide comprising an amino acid sequence selected from SEQ ID NOs 1 to 3, and/or (b) a polypeptide comprising (i) an amino acid sequence that has sequence identity to one or more of SEQ ID NOs 1 to 3 and/or (ii) a fragment of SEQ ID NOs 1 to 3.

Compositions of the invention may also comprise mixtures of these GBS protein antigens.

In particular, compositions of the invention may include:

(a₁) a polypeptide comprising an amino acid sequence of SEQ ID NO 1, and/or (b₁) a polypeptide comprising (i) an amino acid sequence that has sequence identity to SEQ ID NO 1 and/or (ii) a fragment of SEQ ID NO 1; and

(a₂) a polypeptide comprising an amino acid sequence of SEQ ID NO 2, and/or (b₂) a polypeptide comprising (i) an amino acid sequence that has sequence identity to SEQ ID NO 2 and/or (ii) a fragment of SEQ ID NO 2.

Similarly, compositions of the invention may include:

(a₁) a polypeptide comprising an amino acid sequence of SEQ ID NO 1, and/or (b₁) a polypeptide comprising (i) an amino acid sequence that has sequence identity to SEQ ID NO 1 and/or (ii) a fragment of SEQ ID NO 1; and

(a₂) a polypeptide comprising an amino acid sequence of SEQ ID NO 3, and/or (b₂) a polypeptide comprising (i) an amino acid sequence that has sequence identity to SEQ ID NO 3 and/or (ii) a fragment of SEQ ID NO 3.

In the same way, compositions of the invention may include:

(a₁) a polypeptide comprising an amino acid sequence of SEQ ID NO 2, and/or (b₁) a polypeptide comprising (i) an amino acid sequence that has sequence identity to SEQ ID NO 2 and/or (ii) a fragment of SEQ ID NO 2; and

(a₂) a polypeptide comprising an amino acid sequence of SEQ ID NO 3, and/or (b₂) a polypeptide comprising (i) an amino acid sequence that has sequence identity to SEQ ID NO 3 and/or (ii) a fragment of SEQ ID NO 3.

Compositions of the invention may include:

(a₁) a polypeptide comprising an amino acid sequence of SEQ ID NO 1, and/or (b₁) a polypeptide comprising (i) an amino acid sequence that has sequence identity to SEQ ID NO 1 and/or (ii) a fragment of SEQ ID NO 1;

(a₂) a polypeptide comprising an amino acid sequence of SEQ ID NO 2, and/or (b₂) a polypeptide comprising (i) an amino acid sequence that has sequence identity to SEQ ID NO 2 and/or (ii) a fragment of SEQ ID NO 2; and

(a₃) a polypeptide comprising an amino acid sequence of SEQ ID NO 3, and/or (b₃) a polypeptide comprising (i) an amino acid sequence that has sequence identity to SEQ ID NO 3 and/or (ii) a fragment of SEQ ID NO 3.

Three other preferred GBS protein antigens for use with the invention are known as: GBS104; GBS276; and GBS322 [see ref 99]. The wild-type GBS104 amino acid sequence from serotype V isolated strain 2603 V/R is given in reference Error! Bookmark not defined. as SEQ ID NO: 3 therein. Where embodiments of the present invention are defined herein by reference to SEQ ID NO: 1, the references to SEQ ID NO: 1 may be substituted by references to SEQ ID NO: 3 from reference 21. The wild-type GBS276 amino acid sequence from serotype V isolated strain 2603 V/R is given in reference Error! Bookmark not defined. as SEQ ID NO: 4 therein. Where embodiments of the present invention are defined herein by reference to SEQ ID NO: 2, the references to SEQ ID NO: 2 may be substituted by references to SEQ ID NO: 4 from reference Error! Bookmark not defined. The wild-type GBS322 amino acid sequence from serotype V isolated strain 2603 V/R is given in reference Error! Bookmark not defined. as SEQ ID NO: 5 therein. Where embodiments of the present invention are defined herein by reference to SEQ ID NO: 3, the references to SEQ ID NO: 3 may be substituted by references to SEQ ID NO: 5 from reference 21.

Depending on the particular SEQ ID NO, the degree of sequence identity in (i) is preferably greater than 50% (e.g. 60%, 70%, 75%, 80%, 85%, 90%, 91%, 92%, 93%, 94%, 95%, 96%, 97%, 98%, 99% or more). These polypeptides include homologs, orthologs, allelic variants and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty=12 and gap extension penalty=1.

Depending on the particular SEQ ID NO, the fragments of (ii) should comprise at least n consecutive amino acids from the sequences and, depending on the particular sequence, n is 7 or more (e.g. 8, 10, 12, 14, 16, 18, 20, 22, 24, 26, 28, 30, 35, 40, 45, 50, 60, 70, 80, 90, 100 or more). The fragment may comprise at least one T-cell or, preferably, a B-cell epitope of the sequence. T- and B-cell epitopes can be identified empirically (e.g. using PEPSCAN [208,209] or similar methods), or they can be predicted (e.g. using the Jameson-Wolf antigenic index [210], matrix-based approaches [211], TEPITOPE [212], neural networks [213], OptiMer & EpiMer [214, 215], ADEPT [216], Tsites [217], hydrophilicity [218], antigenic index [219] or the methods disclosed in reference 220 etc.). Other preferred fragments are SEQ ID NOs 1 to 3 without their N-terminal amino acid residue or without their N-terminal signal peptide. Removal of one or more domains, such as a leader or signal sequence region, a transmembrane region, a cytoplasmic region or a cell wall anchoring motif can be used. Preferred fragments of a particular protein can bind to an antibody that can bind to the full-length particular protein e.g. can bind to an antibody that binds to SEQ ID NO: 1, 2 or 3. Some useful fragments are given below (SEQ ID NOs 4 to 13).

These polypeptides may, compared to SEQ ID NOs 1 to 3, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cystine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may also include one or more (e.g. 1, 2, 3, 4, 5, 6, 7, 8, 9, 10, etc.) single amino acid deletions relative to SEQ ID NOs 1 to 3. The polypeptides may also include one or more (e.g. 1, 2, 3, 4, 5, 6, 7, 8, 9, 10, etc.) insertions (e.g. each of 1, 2, 3, 4 or 5 amino acids) relative to the SEQ ID NOs 1 to 3.

Polypeptides of the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [221,222]. Solid-phase peptide synthesis is particularly preferred, such as methods based on tBoc or Fmoc [223] chemistry. Enzymatic synthesis [224] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [225]. Where D-amino acids are included, however, it is preferred to use chemical synthesis. Polypeptides of the invention may have covalent modifications at the C-terminus and/or N-terminus.

If these GBS proteins are included in compositions of the invention then they can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.). They are preferably used in purified or substantially purified form i.e. substantially free from other polypeptides (e.g. free from naturally-occurring polypeptides), particularly from other GBS or host cell polypeptides).

GBS67

Nucleotide and amino acid sequence of GBS67 sequenced from serotype V strain 2603 V/R are set forth in ref. Error! Bookmark not defined. as SEQ ID NOs 3745 & 3746. The amino acid sequence is SEQ ID NO:1 herein:

MRKYQKFSKILTLSLFCLSQIPLNTNVLGESTVPENGAKGKLVVKKTDDQ
NKPLSKATFVLKTTAHPESKIEKVTAELTGEATFDNLIPGDYTLSEETAP
EGYKKTNQTWQVKVESNGKTTIQNSGDKNSTIGQNQEELDKQYPPTGIYE
DTKESYKLEHVKGSVPNGKSEAKAVNPYSSEGEHIREIPEGTLSKRISEV
GDLAHNKYKIELTVSGKTIVKPVDKQKPLDVVFVLDNSNSMNNDGPNFQR
HNKAKKAAEALGTAVKDILGANSDNRVALVTYGSDIFDGRSVDVVKGFKE
DDKYYGLQTKFTIQTENYSHKQLTNNAEEIIKRIPTEAPKAKWGSTTNGL
TPEQQKEYYLSKVGETFTMKAFMEADDILSQVNRNSQKIIVHVTDGVPTR
SYAINNFKLGASYESQFEQMKKNGYLNKSNFLLTDKPEDIKGNGESYFLF
PLDSYQTQIISGNLQKLHYLDLNLNYPKGTIYRNGPVKEHGTPTKLYINS
LKQKNYDIFNFGIDISGFRQVYNEEYKKNQDGTFQKLKEEAFKLSDGEIT
ELMRSFSSKPEYYTPIVTSADTSNNEILSKIQQQFETILTKENSIVNGTI
EDPMGDKINLQLGNGQTLQPSDYTLQGNDGSVMKDGIATGGPNNDGGILK
GVKLEYIGNKLYVRGLNLGEGQKVTLTYDVKLDDSFISNKFYDTNGRTTL
NPKSEDPNTLRDFPIPKIRDVREYPTITIKNEKKLGEIEFIKVDKDNNKL
LLKGATFELQEFNEDYKLYLPIKNNNSKVVTGENGKISYKDLKDGKYQLI
EAVSPEDYQKITNKPILTFEVVKGSIKNIIAVNKQISEYHEEGDKHLITN
THIPPKGIIPMTGGKGILSFILIGGAMMSIAGGIYIWKRYKKSSDMSIKK
D

GBS67 contains a C-terminus transmembrane region which is indicated by the underlined region closest to the C-terminus of SEQ ID NO: 1 above. One or more amino acids from the transmembrane region may be removed, or the amino acid may be truncated before the transmembrane region. An example of such a GBS67 fragment is set forth below as SEQ ID NO: 4.

MRKYQKFSKILTLSLFCLSQIPLNTNVLGESTVPENGAKGKLVVKKTDDQ
NKPLSKATFVLKTTAHPESKIEKVTAELTGEATFDNLIPGDYTLSEETAP
EGYKKTNQTWQVKVESNGKTTIQNSGDKNSTIGQNQEELDKQYPPTGIYE
DTKESYKLEHVKGSVPNGKSEAKAVNPYSSEGEHIREIPEGTLSKRISEV
GDLAHNKYKIELTVSGKTIVKPVDKQKPLDVVFVLDNSNSMNNDGPNFQR
HNKAKKAAEALGTAVKDILGANSDNRVALVTYGSDIFDGRSVDVVKGFKE
DDKYYGLQTKFTIQTENYSHKQLTNNAEEIIKRIPTEAPKAKWGSTTNGL
TPEQQKEYYLSKVGETFTMKAFMEADDILSQVNRNSQKIIVHVTDGVPTR
SYAINNFKLGASYESQFEQMKKNGYLNKSNFLLTDKPEDIKGNGESYFLF
PLDSYQTQIISGNLQKLHYLDLNLNYPKGTIYRNGPVKEHGTPTKLYINS
LKQKNYDIFNFGIDISGFRQVYNEEYKKNQDGTFQKLKEEAFKLSDGEIT
ELMRSFSSKPEYYTPIVTSADTSNNEILSKIQQQFETILTKENSIVNGTI
EDPMGDKINLQLGNGQTLQPSDYTLQGNDGSVMKDGIATGGPNNDGGILK
GVKLEYIGNKLYVRGLNLGEGQKVTLTYDVKLDDSFISNKFYDTNGRTTL
NPKSEDPNTLRDFPIPKIRDVREYPTITIKNEKKLGEIEFIKVDKDNNKL
LLKGATFELQEFNEDYKLYLPIKNNNSKVVTGENGKISYKDLKDGKYQLI
EAVSPEDYQKITNKPILTFEVVKGSIKNIIAVNKQISEYHEEGDKHLITN
THIPPKGIIPMTGGKGILS

GBS67 contains an amino acid motif indicative of a cell wall anchor, shown in italics in SEQ ID NO: 1 above. In some recombinant host cell systems, it may be preferable to remove this motif to facilitate secretion of a recombinant GBS67 protein from the host cell. Accordingly, in one preferred fragment of GBS67 for use in the invention, the transmembrane and the cell wall anchor motif are removed from GBS67. An example of such a GBS67 fragment is set forth below as SEQ ID NO: 5.

MRKYQKFSKILTLSLFCLSQIPLNTNVLGESTVPENGAKGKLVVKKTDDQ
NKPLSKATFVLKTTAHPESKIEKVTAELTGEATFDNLIPGDYTLSEETAP
EGYKKTNQTWQVKVESNGKTTIQNSGDKNSTIGQNQEELDKQYPPTGIYE
DTKESYKLEHVKGSVPNGKSEAKAVNPYSSEGEHIREIPEGTLSKRISEV
GDLAHNKYKIELTVSGKTIVKPVDKQKPLDVVFVLDNSNSMNNDGPNFQR
HNKAKKAAEALGTAVKDILGANSDNRVALVTYGSDIFDGRSVDVVKGFKE
DDKYYGLQTKFTIQTENYSHKQLTNNAEEIIKRIPTEAPKAKWGSTTNGL
TPEQQKEYYLSKVGETFTMKAFMEADDILSQVNRNSQKIIVHVTDGVPTR
SYAINNFKLGASYESQFEQMKKNGYLNKSNFLLTDKPEDIKGNGESYFLF
PLDSYQTQIISGNLQKLHYLDLNLNYPKGTIYRNGPVKEHGTPTKLYINS
LKQKNYDIFNFGIDISGFRQVYNEEYKKNQDGTFQKLKEEAFKLSDGEIT
ELMRSFSSKPEYYTPIVTSADTSNNEILSKIQQQFETILTKENSIVNGTI
EDPMGDKINLQLGNGQTLQPSDYTLQGNDGSVMKDGIATGGPNNDGGILK
GVKLEYIGNKLYVRGLNLGEGQKVTLTYDVKLDDSFISNKFYDTNGRTTL
NPKSEDPNTLRDFPIPKIRDVREYPTITIKNEKKLGEIEFIKVDKDNNKL
LLKGATFELQEFNEDYKLYLPIKNNNSKVVTGENGKISYKDLKDGKYQLI
EAVSPEDYQKITNKPILTFEVVKGSIKNIIAVNKQISEYHEEGDKHLITN
THIPPKGI

Alternatively, in some recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Three pilin motifs, containing conserved lysine residues have been identified in GBS67. Conserved lysine residues are at amino acid residues 478 and 488, at amino acid residues 340 and 342, and at amino acid residues 703 and 717. The pilin sequences, in particular the conserved lysine residues, are thought to be important for the formation of oligomeric, pilus-like structures of GBS67. Preferred fragments of GBS67 include at least one conserved lysine residue. Two E boxes containing conserved glutamic residues have also been identified in GBS67. Preferred fragments of GBS67 include at least one conserved glutamic acid residue. GBS67 contains several regions predicted to form alpha helical structures. Such alpha helical regions are likely to form coiled-coil structures and may be involved in oligomerization of GBS67. GBS67 also contains a region which is homologous to the Cna B domain of the S. aureus collagen-binding surface protein (pfam05738). This may form a beta sandwich structure. GBS67 contains a region which is homologous to a von Willebrand factor (vWF) type A domain.

The amino acid sequence of GBS67 sequenced from serotype Ib strain H36B is set forth in ref 226 as SEQ ID NO 20906. The amino acid sequence is SEQ ID NO: 24 herein:

MRKYQKFSKILTLSLFCLSQIPLNTNVLGESTVPENGAKGKLVVKKTDDQ
NKPLSKATFVLKPTSHSESKVEKVTTEVTGEATFDNLTPGDYTLSEETAP
EGYKKTTQTWQVKVESNGKTTIQNSDDKKSIIEQRQEELDKQYPLTGAYE
DTKESYNLEHVKNSIPNGKLEAKAVNPYSSEGEHIREIQEGTLSKRISEV
NDLDHNKYKIELTVSGKSIIKTINKDEPLDVVFVLDNSNSMKNNGKNNKA
KKAGEAVETIIKDVLGANVENRAALVTYGSDIFDGRTVKVIKGFKEDPYY
GLETSFTVQTNDYSYKKFTNIAADIIKKIPKEAPEAKWGGTSLGLTPEKK
REYDLSKVGETFTMKAFMEADTLLSSIQRKSRKIIVHLTDGVPTRSYAIN
SFVKGSTYANQFERIKEKGYLDKNNYFITDDPEKIKGNGESYFLFPLDSY
QTQIISGNLQKLHYLDLNLNYPKGTIYRNGPVREHGTPTKLYINSLKQKN
YDIFNFGIDISGFRQVYNEDYKKNQDGTFQKLKEEAFELSDGEITELMNS
FSSKPEYYTPIVTSADVSNNEILSKIQQQFEKILTKENSIVNGTIEDPMG
DKINLHLGNGQTLQPSDYTLQGNDGSIMKDSIATGGPNNDGGILKGVKLE
YIKNKLYVRGLNLGEGQKVTLTYDVKLDDSFISNKFYDTNGRTTLNPKSE
EPDTLRDFPIPKIRDVREYPTITIKNEKKLGEIEFTKVDKDNNKLLLKGA
TFELQEFNEDYKLYLPIKNNNSKVVTGENGKISYKDLKDGKYQLIEAVSP
KDYQKITNKPETFEVVKGSIQNIIAVNKQISEYHEEGDKHLITNTHIPPK
GIIPMTGGKGILSFILIGGAMMSIAGGIYIWKRHKKSSDASIEKD

In some embodiments, this variant of GBS67 may be used. Accordingly, where embodiments of the present invention are defined herein by reference to SEQ ID NO: 1, the references to SEQ ID NO: 1 may be substituted by references to SEQ ID NO: 24.

Like GBS67 sequenced from serotype V strain 2603 V/R, GBS67 sequenced from serotype Ib strain H36B contains a C-terminus transmembrane region which is indicated by the underlined region closest to the C-terminus of SEQ ID NO: 24 above. One or more amino acids from the transmembrane region may be removed, or the amino acid may be truncated before the transmembrane region. An example of such a GBS67 fragment is set forth below as SEQ ID NO: 25.

MRKYQKFSKILTLSLFCLSQIPLNTNVLGESTVPENGAKGKLVVKKTDDQ
NKPLSKATFVLKPTSHSESKVEKVTTEVTGEATFDNLTPGDYTLSEETAP
EGYKKTTQTWQVKVESNGKTTIQNSDDKKSIIEQRQEELDKQYPLTGAYE
DTKESYNLEHVKNSIPNGKLEAKAVNPYSSEGEHIREIQEGTLSKRISEV
NDLDHNKYKIELTVSGKSIIKTINKDEPLDVVFVLDNSNSMKNNGKNNKA
KKAGEAVETIIKDVLGANVENRAALVTYGSDIFDGRTVKVIKGFKEDPYY
GLETSFTVQTNDYSYKKFTNIAADIIKKIPKEAPEAKWGGTSLGLTPEKK
REYDLSKVGETFTMKAFMEADTLLSSIQRKSRKIIVHLTDGVPTRSYAIN
SFVKGSTYANQFERIKEKGYLDKNNYFITDDPEKIKGNGESYFLFPLDSY
QTQIISGNLQKLHYLDLNLNYPKGTIYRNGPVREHGTPTKLYINSLKQKN
YDIFNFGIDISGFRQVYNEDYKKNQDGTFQKLKEEAFELSDGEITELMNS
FSSKPEYYTPIVTSADVSNNEILSKIQQQFEKILTKENSIVNGTIEDPMG
DKINLHLGNGQTLQPSDYTLQGNDGSIMKDSIATGGPNNDGGILKGVKLE
YIKNKLYVRGLNLGEGQKVTLTYDVKLDDSFISNKFYDTNGRTTLNPKSE
EPDTLRDFPIPKIRDVREYPTITIKNEKKLGEIEFTKVDKDNNKLLLKGA
TFELQEFNEDYKLYLPIKNNNSKVVTGENGKISYKDLKDGKYQLIEAVSP
KDYQKITNKPILTFEVVKGSIQNIIAVNKQISEYHEEGDKHLITNTHIPP
KGIIPMTGGKGILS

Like GBS67 sequenced from serotype V strain 2603 V/R, GBS67 sequenced from serotype Ib strain H36B contains an amino acid motif indicative of a cell wall anchor, shown in italics in SEQ ID NO: 24 above. Accordingly, in one preferred fragment of GBS67 for use in the invention, the transmembrane and the cell wall anchor motif are removed from GBS67. An example of such a GBS67 fragment is set forth below as SEQ ID NO: 26.

MRKYQKFSKILTLSLFCLSQIPLNTNVLGESTVPENGAKGKLVVKKTDDQ
NKPLSKATFVLKPTSHSESKVEKVTTEVTGEATFDNLTPGDYTLSEETAP
EGYKKTTQTWQVKVESNGKTTIQNSDDKKSIIEQRQEELDKQYPLTGAYE
DTKESYNLEHVKNSIPNGKLEAKAVNPYSSEGEHIREIQEGTLSKRISEV
NDLDHNKYKIELTVSGKSIIKTINKDEPLDVVFVLDNSNSMKNNGKNNKA
KKAGEAVETIIKDVLGANVENRAALVTYGSDIFDGRTVKVIKGFKEDPYY
GLETSFTVQTNDYSYKKFTNIAADIIKKIPKEAPEAKWGGTSLGLTPEKK
REYDLSKVGETFTMKAFMEADTLLSSIQRKSRKIIVHLTDGVPTRSYAIN
SFVKGSTYANQFERIKEKGYLDKNNYFITDDPEKIKGNGESYFLFPLDSY
QTQIISGNLQKLHYLDLNLNYPKGTIYRNGPVREHGTPTKLYINSLKQKN
YDIFNFGIDISGFRQVYNEDYKKNQDGTFQKLKEEAFELSDGEITELMNS
FSSKPEYYTPIVTSADVSNNEILSKIQQQFEKILTKENSIVNGTIEDPMG
DKINLHLGNGQTLQPSDYTLQGNDGSIMKDSIATGGPNNDGGILKGVKLE
YIKNKLYVRGLNLGEGQKVTLTYDVKLDDSFISNKFYDTNGRTTLNPKSE
EPDTLRDFPIPKIRDVREYPTITIKNEKKLGEIEFTKVDKDNNKLLLKGA
TFELQEFNEDYKLYLPIKNNNSKVVTGENGKISYKDLKDGKYQLIEAVSP
KDYQKITNKPILTNEVVKGSIQNIIAVNKQISEYHEEGDKHLITNTHIPP
KGI

GBS80

GBS80 refers to a putative cell wall surface anchor family protein. Nucleotide and amino acid sequence of GBS80 sequenced from serotype V isolated strain 2603 V/R are set forth in ref 99 as SEQ ID NOs 8779 & 8780. The amino acid sequence is set forth below as SEQ ID NO: 2:

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPIKLKSHTDGTFEIK
GLAYAVDANAEGTAVTYKLKETKAPEGYVIPDKEIEFTVSQTSYNTKPTD
ITVDSADATPDTIKNNKRPSIPNTGGIGTAIFVAIGAAVMAFAVKGMKRR
TKDN

GBS80 contains a N-terminal leader or signal sequence region which is indicated by the underlined sequence above. One or more amino acids from the leader or signal sequence region of GBS80 can be removed. An example of such a GBS80 fragment is set forth below as SEQ ID NO: 6:

AEVSQERPAKTTVNIYKLQADSYKSEITSNGGIENKDGEVISNYAKLGDN
VKGLQGVQFKRYKVKTDISVDELKKLTTVEAADAKVGTILEEGVSLPQKT
NAQGLVVDALDSKSNVRYLYVEDLKNSPSNITKAYAVPFVLELPVANSTG
TGFLSEINIYPKNVVTDEPKTDKDVKKLGQDDAGYTIGEEFKWFLKSTIP
ANLGDYEKFEITDKFADGLTYKSVGKIKIGSKTLNRDEHYTIDEPTVDNQ
NTLKITFKPEKFKEIAELLKGMTLVKNQDALDKATANTDDAAFLEIPVAS
TINEKAVLGKAIENTFELQYDHTPDKADNPKPSNPPRKPEVHTGGKRFVK
KDSTETQTLGGAEFDLLASDGTAVKWTDALIKANTNKNYIAGEAVTGQPI
KLKSHTDGTFEIKGLAYAVDANAEGTAVTYKLKETKAPEGYVIPDKEIEF
TVSQTSYNTKPTDITVDSADATPDTIKNNKRPSIPNTGGIGTAIFVAIGA
AVMAFAVKGMKRRTKDN

GBS80 contains a C-terminal transmembrane region which is indicated by the underlined sequence near the end of SEQ ID NO: 2 above. One or more amino acids from the transmembrane region and/or a cytoplasmic region may be removed. An example of such a fragment is set forth below as SEQ ID NO:7:

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPIKLKSHTDGTFEIK
GLAYAVDANAEGTAVTYKLKETKAPEGYVIPDKEIEFTVSQTSYNTKPTD
ITVDSADATPDTIKNNKRPSIPNTG

GBS80 contains an amino acid motif indicative of a cell wall anchor, shown in italics in SEQ ID NO: 2 above. In some recombinant host cell systems, it may be preferable to remove this motif to facilitate secretion of a recombinant GBS80 protein from the host cell. Thus the transmembrane and/or cytoplasmic regions and the cell wall anchor motif may be removed from GBS80. An example of such a fragment is set forth below as SEQ ID NO: 8.

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPIKLKSHTDGTFEIK
GLAYAVDANAEGTAVTYKLKETKAPEGYVIPDKEIEFTVSQTSYNTKPTD
ITVDSADATPDTIKNNKRPS

Alternatively, in some recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

In one embodiment, the leader or signal sequence region, the transmembrane and cytoplasmic regions and the cell wall anchor motif are removed from the GBS80 sequence. An example of such a GBS80 fragment is set forth below as SEQ ID NO: 9:

AEVSQERPAKTTVNIYKLQADSYKSEITSNGGIENKDGEVISNYAKLGDN
VKGLQGVQFKRYKVKTDISVDELKKLTTVEAADAKVGTILEEGVSLPQKT
NAQGLVVDALDSKSNVRYLYVEDLKNSPSNITKAYAVPFVLELPVANSTG
TGFLSEINIYPKNVVTDEPKTDKDVKKLGQDDAGYTIGEEFKWFLKSTIP
ANLGDYEKFEITDKFADGLTYKSVGKIKIGSKTLNRDEHYTIDEPTVDNQ
NTLKITFKPEKFKEIAELLKGMTLVKNQDALDKATANTDDAAFLEIPVAS
TINEKAVLGKAIENTFELQYDHTPDKADNPKPSNPPRKPEVHTGGKRFVK
KDSTETQTLGGAEFDLLASDGTAVKWTDALIKANTNKNYIAGEAVTGQPI
KLKSHTDGTFEIKGLAYAVDANAEGTAVTYKLKETKAPEGYVIPDKEIEF
TVSQTSYNTKPTDITVDSADATPDTIKNNKRPS

A particularly immunogenic fragment of GBS80 is located towards the N-terminus of the protein, and is given herein as SEQ ID NO: 10:

AEVSQERPAKTTVNIYKLQADSYKSEITSNGGIENKDGEVISNYAKLGDN
VKGLQGVQFKRYKVKTDISVDELKKLTTVEAADAKVGTILEEGVSLPQKT
NAQGLVVDALDSKSNVRYLYVEDLKNSPSNITKAYAVPFVLELPVANSTG
TGFLSEINIYPKNVVTDEPKTDKDVKKLGQDDAGYTIGEEFKWFLKSTIP
ANLGDYEKFEITDKFADGLTYKSVGKIKIGSKTLNRDEHYTIDEPTVDNQ
NTLKITFKPEKFKEIAELLKG

Spb1

The wild-type SpbI sequence from serotype III strain COH1 is SEQ ID NO: 3 herein:

MKKKMIQSLLVASLAFGMAVSPVTPIAFAAETGTITVQDTQKGATYKAYK
VFDAEIDNANVSDSNKDGASYLIPQGKEAEYKASTDFNSLFTTTTNGGRT
YVTKKDTASANEIATWAKSISANTTPVSTVTESNNDGTEVINVSQYGYYY
VSSTVNNGAVIMVTSVTPNATIHEKNTDATWGDGGGKTVDQKTYSVGDTV
KYTITYKNAVNYHGTEKVYQYVIKDTMPSASVVDLNEGSYEVTITDGSGN
ITTLTQGSEKATGKYNLLEENNNFTITIPWAATNTPTGNTQNGANDDFFY
KGINTITVTYTGVLKSGAKPGSADLPENTNIATINPNTSNDDPGQKVTVR
DGQITIKKIDGSTKASLQGAIFVLKNATGQFLNFNDTNNVEWGTEANATE
YTTGADGIITITGLKEGTYYLVEKKAPLGYNLLDNSQKVILGDGATDTTN
SDNLLVNPTVENNKGTELPSTGGIGTTIFYIIGAILVIGAGIVLVARRRL
RS

Wild-type SpbI contains a N-terminal leader or signal sequence region which is indicated by the underlined sequence above (aa 1-29). One or more amino acids from the leader or signal sequence region of Spbl can be removed. An example of such a Spbl fragment is set forth below as SEQ ID NO: 11:

AETGTITVQDTQKGATYKAYKVFDAEIDNANVSDSNKDGASYLIPQGKEA
EYKASTDFNSLFTTTTNGGRTYVTKKDTASANEIATWAKSISANTTPVST
VTESNNDGTEVINVSQYGYYYVSSTVNNGAVIMVTSVTPNATIHEKNTDA
TWGDGGGKTVDQKTYSVGDTVKYTITYKNAVNYHGTEKVYQYVIKDTMPS
ASVVDLNEGSYEVTITDGSGNITTLTQGSEKATGKYNLLEENNNFTITIP
WAATNTPTGNTQNGANDDFFYKGINTITVTYTGVLKSGAKPGSADLPENT
NIATINPNTSNDDPGQKVTVRDGQITIKKIDGSTKASLQGAIFVLKNATG
QFLNFNDTNNVEWGTEANATEYTTGADGIITITGLKEGTYYLVEKKAPLG
YNLLDNSQKVILGDGATDTTNSDNLLVNPTVENNKGTELPSTGGIGTTIF
YIIGAILVIGAGIVLVARRRLRS

The wild-type SpbI sequence contains an amino acid motif indicative of a cell wall anchor (LPSTG). In some recombinant host cell systems, it may be preferable to remove this motif to facilitate secretion of a recombinant SpbI protein from the host cell. Thus the cell wall anchor motif and sequence C-terminal to this motif may be removed from SpbI. An example of such a fragment is set forth below as SEQ ID NO: 12:

MKKKMIQSLLVASLAFGMAVSPVTPIAFAAETGTITVQDTQKGATYKAYK
VFDAEIDNANVSDSNKDGASYLIPQGKEAEYKASTDFNSLFTTTTNGGRT
YVTKKDTASANEIATWAKSISANTTPVSTVTESNNDGTEVINVSQYGYYY
VSSTVNNGAVIMVTSVTPNATIHEKNTDATWGDGGGKTVDQKTYSVGDTV
KYTITYKNAVNYHGTEKVYQYVIKDTMPSASVVDLNEGSYEVTITDGSGN
ITTLTQGSEKATGKYNLLEENNNFTITIPWAATNTPTGNTQNGANDDFFY
KGINTITVTYTGVLKSGAKPGSADLPENTNIATINPNTSNDDPGQKVTVR
DGQITIKKIDGSTKASLQGAIFVLKNATGQFLNFNDTNNVEWGTEANATE
YTTGADGIITITGLKEGTYYLVEKKAPLGYNLLDNSQKVILGDGATDTTN
SDNLLVNPTVENNKGTE

Alternatively, in some recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

In one embodiment, the leader or signal sequence region, the cell wall anchor motif and sequence C-terminal to this motif are removed from Spbl. An example of such a Spbl fragment is set forth below as SEQ ID NO: 13:

AETGTITVQDTQKGATYKAYKVFDAEIDNANVSDSNKDGASYLIPQGKEA
EYKASTDFNSLFTTTTNGGRTYVTKKDTASANEIATWAKSISANTTPVST
VTESNNDGTEVINVSQYGYYYVSSTVNNGAVIMVTSVTPNATIHEKNTDA
TWGDGGGKTVDQKTYSVGDTVKYTITYKNAVNYHGTEKVYQYVIKDTMPS
ASVVDLNEGSYEVTITDGSGNITTLTQGSEKATGKYNLLEENNNFTITIP
WAATNTPTGNTQNGANDDFFYKGINTITVTYTGVLKSGAKPGSADLPENT
NIATINPNTSNDDPGQKVTVRDGQITIKKIDGSTKASLQGAIFVLKNATG
QFLNFNDTNNVEWGTEANATEYTTGADGIITITGLKEGTYYLVEKKAPLG
YNLLDNSQKVILGDGATDTTNSDNLLVNPTVENNKGTE

An E box containing a conserved glutamic residue has also been identified in Spbl (underlined), with a conserved glutamic acid at residue 423 (bold). The E box motif may be important for the formation of oligomeric pilus-like structures, and so useful fragments of Spbl may include the conserved glutamic acid residue.

The wild-type Spb1 sequence includes an internal methionine codon (Met-162) that has an upstream 12-mer TAATGGAGCTGT sequence (SEQ ID NO: 14) that includes the core sequence (underlined) of a Shine-Dalgarno sequence. This Shine-Dalgarno sequence has been found to initiate translation of a truncated Spbl sequence. To prevent translation initiation at this site the Shine-Dalgarno sequence can be disrupted in a Spb 1-coding sequence used for expression. Although any suitable nucleotide can be mutated to prevent ribosome binding, the sequence includes a GGA glycine codon that is both part of the Shine-Dalgarno core and in-frame with the internal methionine codon. The third base in this codon can be mutated to C, G or T without changing the encoded glycine, thereby avoiding any change in Spbl sequence.

Compositions of the invention may also include a polypeptide defined in refererence [227] by the amino acid sequence NH₂—W—X—L—Y—Z—CO₂H, wherein: X is a Spb1 sequence; L is an optional linker; and Y is a GBS80 sequence; W is an optional N-terminal sequence; and Z is an optional C-terminal sequence. Further details of this polypeptide are given below.

These compositions may also comprise one or more of the GBS protein antigens described above. In particular, compositions of the invention may include (a) a polypeptide of amino acid sequence NH₂—W—X—L—Y—Z—CO₂H; and (b₁) a polypeptide comprising an amino acid sequence of SEQ ID NO 1 as described above, and/or (b₂) a polypeptide comprising (i) an amino acid sequence that has sequence identity to SEQ ID NO 1 as described above and/or (ii) a fragment of SEQ ID NO 1 as described above.

Polypeptide NH₂—W—X—L—Y—Z—CO₂H

Typically, the polypeptide comprises an amino acid sequence X—L—Y, wherein: X is a Spb1 sequence; L is an optional linker; and Y is a GBS80 sequence.

X: Spb1 Sequence

The X moiety is a Spb1 sequence. This Spb1 sequence will, when administered to a subject, elicit an antibody response comprising antibodies that bind to wild-type Spb1 protein e.g. to the S.agalactiae protein having amino acid sequence SEQ ID NO: 3 (the full-length wild-type sequence from strain COH1).

The Spb1 sequence may comprise an amino acid sequence having at least a% identity to SEQ ID NO: 13. The value of a may be selected from 50, 60, 70, 80, 85, 90, 95, 96, 97, 98, 99 or more. The Spb1 sequence may comprise SEQ ID NO: 13.

The Spb1 sequence may comprise a fragment of SEQ ID NO: 3 and/or of SEQ ID NO:13. The fragment will usually include at least b amino acids of SEQ ID NO: 3/13, wherein b is selected from 5, 6, 7, 8, 9, 10, 11, 12, 13, 14, 15, 16, 17, 18, 19, 20, 22, 24, 26, 28, 30, 40, 50, 60, 70, 80, 90, 100, 110, 120, 130, 140, 150, 175, 200 or more. The fragment will usually include at least one T-cell or, preferably, a B-cell epitope of SEQ ID NO: 3/13. T- and B- cell epitopes can be identified by the methods described above. SEQ ID NO: 13 is itself a fragment of SEQ ID NO: 3, as explained above.

The Spb1 sequence may comprise an amino acid sequence that has both at least a% identity to SEQ ID NO: 13 and comprises a fragment of SEQ ID NO: 13, as defined above.

The X moiety will usually be at least c amino acids long, where c is selected from 50, 60, 70, 80, 90, 100, 120, 140, 160, 180, 200, 220, 240, 260, 280, 300, 320, 340, 360, 380, 400 or more.

The X moiety will usually be no longer than d amino acids long, where d is selected from 500, 480, 460, 440, 420, 400, 380, 360, 340, 320, 300, 280, 260, 240, 220, 200 or less.

The X moiety will usually be between 300-500 amino acids long e.g. 350-480, 400-460, 430-450.

The wild-type Spb1 sequence from serotype III strain COH1 is SEQ ID NO: 3 above. The specific derivatives thereof described in section “SpbI” above are applicable to the Spb1 sequence of this embodiment of the invention.

Y: GBS80 Sequence

The Y moiety is a GBS80 sequence. This GBS80 sequence will, when administered to a subject, elicit an antibody response comprising antibodies that bind to wild-type GBS80 protein e.g. to the S.agalactiae protein having amino acid sequence SEQ ID NO: 2 (the full-length wild-type sequence from strain 2603V/R).

The GBS80 sequence may comprise an amino acid sequence having at least e% identity to SEQ ID NO: 9. The value of e may be selected from 50, 60, 70, 80, 85, 90, 95, 96, 97, 98, 99 or more. The GBS80 sequence may comprise SEQ ID NO: 9.

The GBS80 sequence may comprise a fragment of SEQ ID NO: 2 or of SEQ ID NO: 9. The fragment will usually include at least f amino acids of SEQ ID NO: 2/9, wherein fis selected from 5, 6, 7, 8, 9, 10, 11, 12, 13, 14, 15, 16, 17, 18, 19, 20, 22, 24, 26, 28, 30, 40, 50, 60, 70, 80, 90, 100, 110, 120, 130, 140, 150, 175, 200 or more. The fragment will usually include at least one T-cell or, preferably, B-cell epitope of SEQ ID NO: 2/9. SEQ ID NO: 9 is itself a fragment of SEQ ID NO: 2, as explained above.

The GBS80 sequence may comprise an amino acid sequence that has both at least e% identity to SEQ ID NO: 9 and comprises a fragment of SEQ ID NO: 9, as defined above.

The Y moiety will usually be at least g amino acids long, where g is selected from 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600 or more.

The Y moiety will usually be no longer than h amino acids long, where h is selected from 600, 580, 560, 540, 520, 500, 480, 460, 440, 420, 400, 380, 360, 340, 320, 300, 280, 260, 240, 220, 200 or less.

The Y moiety will usually be between 350-550 amino acids long e.g. 400-520, 450-500, 470-490.

The wild-type GBS80 sequence from serotype V isolated strain 2603 V/R is SEQ ID NO: 2 above. The specific derivatives thereof described in section “GBS80” above are applicable to the GBS80 sequence of this embodiment of the invention.

L: Linker

The polypeptide optionally includes a L moiety to link the X and Y moieties. The L moiety is typically a short amino acid sequence e.g. in the range of 2-40 amino acids e.g. consisting of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids.

Linkers will usually contain at least one glycine residue, thereby facilitating structural flexibility. The linker may contain, for example, 1, 2, 3, 4, 5, 6, 7, 8, 9, 10 or more glycine residues. The glycines may be arranged to include at least two consecutive glycines in a Gly-Gly dipeptide sequence, or a longer oligo-Gly sequence i.e. Glyₙ where n=2, 3, 4, 5, 6, 7, 8, 9, 10 or more, e.g. SEQ ID NO: 15:

GGGG

Linkers may be encoded by codons found in the recognition sequences of restriction enzymes.

For example, a 6-mer sequence that is the target of a particular restriction enzyme can code for a dipeptide. Thus the recognition sequence for BamHI (GGATCC) encodes Gly-Ser, and so a linker may include a Gly-Ser dipeptide sequence. Such sequences facilitate cloning and manipulation.

Useful linker sequences include SEQ ID NO 15 above and SEQ ID NOs 16, 17 and 18 below:

(SEQ ID NO: 16)

GGGGSGGGGSGGGG

(SEQ ID NO: 17)

GGGGSGGGGSGGGGSEL

(SEQ ID NO: 18)

GSGGGG

However, preferred linkers do not include a sequence that shares 10 or more contiguous amino acids in common with a human polypeptide sequence. For instance, one glycine-rich linker sequence that can be used with the invention is the 14mer SEQ ID NO: 16. However, this 14mer is also found in a human RNA binding protein (gi: 8051631) and so it is preferably avoided within the L moiety.

W: N-Terminal Sequence

The X moiety may be at the N-terminus of the polypeptide, but it is also possible to have amino acids upstream of X. These optional amino acids form a W moiety.

The W moiety is typically a short amino acid sequence e.g. in the range of 2-40 amino acids e.g. consisting of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids.

Examples of W moieties are leader sequences to direct protein trafficking, or comprise short peptide sequences which facilitate cloning or purification (e.g. histidine tags i.e. Hisₙ where n =3, 4, 5, 6, 7, 8, 9, 10 or more). Other suitable N-terminal amino acid sequences will be apparent to those skilled in the art.

In a nascent polypeptide the W moiety can provide the polypeptide's N-terminal methionine (formyl-methionine, fMet, in bacteria). One or more amino acids may be cleaved from the N-terminus of a nascent W moiety, however, such that the W moiety in a polypeptide of the invention does not necessarily include a N-terminal methionine.

Useful W moieties include SEQ ID NO 19:

MAS

Z: C-Terminal Sequence

The Y moiety may be at the C-terminus of the polypeptide, but it is also possible to have amino acids downstream of Y. These optional amino acids form a Z moiety.

The Z moiety is typically a short amino acid sequence e.g. in the range of 2-40 amino acids e.g. consisting of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids.

Examples of Z moieties include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminal amino acid sequences will be apparent to those skilled in the art, such as a glutathione-S-transferase, thioredoxin, 14 kDa fragment of S.aureus protein A, a biotinylated peptide, a maltose-binding protein, an enterokinase flag, etc. One useful Z moiety comprises SEQ ID NO 20:

HHHHHH

Useful Combinations

Of the various X, Y and L moieties, useful combinations include, but are not limited to:

| SEQ ID | W | X | L | Y | Z |
|---|---|---|---|---|---|
| 21 | 19 | 13 | 17 | 9 | — |
| 22 | 19 | 13 | 17 | 9 | 20 |
| 23 | 19 | 13 | 18 | 9 | — |

(SEQ ID NO: 21)

MASAETGTITVQDTQKGATYKAYKVFDAEIDNANVSDSNKDGASYLIPQG
KEAEYKASTDFNSLFTTTTNGGRTYVTKKDTASANEIATWAKSISANTTP
VSTVTESNNDGTEVINVSQYGYYYVSSTVNNGAVIMVTSVTPNATIHEKN
TDATWGDGGGKTVDQKTYSVGDTVKYTITYKNAVNYHGTEKVYQYVIKDT
MPSASVVDLNEGSYEVTITDGSGNITTLTQGSEKATGKYNLLEENNNFTI
TIPWAATNTPTGNTQNGANDDFFYKGINTITVTYTGVLKSGAKPGSADLP
ENTNIATINPNTSNDDPGQKVTVRDGQITIKKIDGSTKASLQGAIFVLKN
ATGQFLNFNDTNNVEWGTEANATEYTTGADGIITITGLKEGTYYLVEKKA
PLGYNLLDNSQKVILGDGATDTTNSDNLLVNPTVENNKGTEGGGGSGGGG
SGGGGSELAEVSQERPAKTTVNIYKLQADSYKSEITSNGGIENKDGEVIS
NYAKLGDNVKGLQGVQFKRYKVKTDISVDELKKLTTVEAADAKVGTILEE
GVSLPQKTNAQGLVVDALDSKSNVRYLYVEDLKNSPSNITKAYAVPFVLE
LPVANSTGTGFLSEINIYPKNVVTDEPKTDKDVKKLGQDDAGYTIGEEFK
WFLKSTIPANLGDYEKFEITDKFADGLTYKSVGKIKIGSKTLNRDEHYTI
DEPTVDNQNTLKITFKPEKFKEIAELLKGMTLVKNQDALDKATANTDDAA
FLEIPVASTINEKAVLGKAIENTFELQYDHTPDKADNPKPSNPPRKPEVH
TGGKRFVKKDSTETQTLGGAEFDLLASDGTAVKWTDALIKANTNKNYIAG
EAVTGQPIKLKSHTDGTFEIKGLAYAVDANAEGTAVTYKLKETKAPEGYV
IPDKEIEFTVSQTSYNTKPTDITVDSADATPDTIKNNKRPS

(SEQ ID NO: 22)

MASAETGTITVQDTQKGATYKAYKVFDAEIDNANVSDSNKDGASYLIPQG
KEAEYKASTDFNSLFTTTTNGGRTYVTKKDTASANEIATWAKSISANTTP
VSTVTESNNDGTEVINVSQYGYYYVSSTVNNGAVIMVTSVTPNATIHEKN
TDATWGDGGGKTVDQKTYSVGDTVKYTITYKNAVNYHGTEKVYQYVIKDT
MPSASVVDLNEGSYEVTITDGSGNITTLTQGSEKATGKYNLLEENNNFTI
TIPWAATNTPTGNTQNGANDDFFYKGINTITVTYTGVLKSGAKPGSADLP
ENTNIATINPNTSNDDPGQKVTVRDGQITIKKIDGSTKASLQGAIFVLKN
ATGQFLNFNDTNNVEWGTEANATEYTTGADGIITITGLKEGTYYLVEKKA
PLGYNLLDNSQKVILGDGATDTTNSDNLLVNPTVENNKGTEGGGGSGGGG
SGGGGSELAEVSQERPAKTTVNIYKLQADSYKSEITSNGGIENKDGEVIS
NYAKLGDNVKGLQGVQFKRYKVKTDISVDELKKLTTVEAADAKVGTILEE
GVSLPQKTNAQGLVVDALDSKSNVRYLYVEDLKNSPSNITKAYAVPFVLE
LPVANSTGTGFLSEINIYPKNVVTDEPKTDKDVKKLGQDDAGYTIGEEFK
WFLKSTIPANLGDYEKFEITDKFADGLTYKSVGKIKIGSKTLNRDEHYTI
DEPTVDNQNTLKITFKPEKFKEIAELLKGMTLVKNQDALDKATANTDDAA
FLEIPVASTINEKAVLGKAIENTFELQYDHTPDKADNPKPSNPPRKPEVH
TGGKRFVKKDSTETQTLGGAEFDLLASDGTAVKWTDALIKANTNKNYIAG
EAVTGQPIKLKSHTDGTFEIKGLAYAVDANAEGTAVTYKLKETKAPEGYV
IPDKEIEFTVSQTSYNTKPTDITVDSADATPDTIKNNKRPSHHHHHH

(SEQ ID NO: 23)

MASAETGTITVQDTQKGATYKAYKVFDAEIDNANVSDSNKDGASYLIPQG
KEAEYKASTDFNSLFTTTTNGGRTYVTKKDTASANEIATWAKSISANTTP
VSTVTESNNDGTEVINVSQYGYYYVSSTVNNGAVIMVTSVTPNATIHEKN
TDATWGDGGGKTVDQKTYSVGDTVKYTITYKNAVNYHGTEKVYQYVIKDT
MPSASVVDLNEGSYEVTITDGSGNITTLTQGSEKATGKYNLLEENNNFTI
TIPWAATNTPTGNTQNGANDDFFYKGINTITVTYTGVLKSGAKPGSADLP
ENTNIATINPNTSNDDPGQKVTVRDGQITIKKIDGSTKASLQGAIFVLKN
ATGQFLNFNDTNNVEWGTEANATEYTTGADGIITITGLKEGTYYLVEKKA
PLGYNLLDNSQKVILGDGATDTTNSDNLLVNPTVENNKGTEGSGGGGELA
EVSQERPAKTTVNIYKLQADSYKSEITSNGGIENKDGEVISNYAKLGDNV
KGLQGVQFKRYKVKTDISVDELKKLTTVEAADAKVGTILEEGVSLPQKTN
AQGLVVDALDSKSNVRYLYVEDLKNSPSNITKAYAVPFVLELPVANSTGT
GFLSEINIYPKNVVTDEPKTDKDVKKLGQDDAGYTIGEEFKWFLKSTIPA
NLGDYEKFEITDKFADGLTYKSVGKIKIGSKTLNRDEHYTIDEPTVDNQN
TLKITFKPEKFKEIAELLKGMTLVKNQDALDKATANTDDAAFLEIPVAST
INEKAVLGKAIENTFELQYDHTPDKADNPKPSNPPRKPEVHTGGKRFVKK
DSTETQTLGGAEFDLLASDGTAVKWTDALIKANTNKNYIAGEAVTGQPIK
LKSHTDGTFEIKGLAYAVDANAEGTAVTYKLKETKAPEGYVIPDKEIEFT
VSQTSYNTKPTDITVDSADATPDTIKNNKRPS

The polypeptide may comprise an amino acid sequence having at least i% sequence identity to SEQ ID NO: 21. The value of i may be selected from 50, 60, 70, 80, 85, 90, 95, 96, 97, 98, 99 or more. The polypeptide may comprise SEQ ID NO: 21.

The polypeptide may comprise an amino acid sequence having at least i% sequence identity to SEQ ID NO: 23. The value of i may be selected from 50, 60, 70, 80, 85, 90, 95, 96, 97, 98, 99 or more. The polypeptide may comprise SEQ ID NO: 23.

A polypeptide used with the invention may comprise an amino acid sequence that:
(a) is identical (i.e. 100% identical) to SEQ ID NO: 21 or 23;
(b) shares sequence identity SEQ ID NO: 21 or 23;
(c) has 1, 2, 3, 4, 5, 6, 7, 8, 9 or 10 (or more) single amino acid alterations (deletions, insertions, substitutions), which may be at separate locations or may be contiguous, as compared to the sequences of (a) or (b); and
(d) when aligned SEQ ID 21 or 23 using a pairwise alignment algorithm, each moving window of x amino acids from N-terminus to C-terminus (such that for an alignment that extends to p amino acids, where p>x, there are p−x+1 such windows) has at least xy identical aligned amino acids, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if xy is not an integer then it is rounded up to the nearest integer. The preferred pairwise alignment algorithm is the Needleman-Wunsch global alignment algorithm [228], using default parameters (e.g. with Gap opening penalty=10.0, and with Gap extension penalty=0.5, using the EBLOSUM62 scoring matrix). This algorithm is conveniently implemented in the needle tool in the EMBOSS package [229].

Within group (c), deletions or substitutions may be at the N-terminus and/or C-terminus, or may be between the two termini. Thus a truncation is an example of a deletion. Truncations may involve deletion of up to 40 (or more) amino acids at the N-terminus and/or C-terminus.

The Spb1 and GBS80 sequences in the polypeptides may be derived from one or more GBS strains. For instance, SEQ ID NOs: 21 and 23 include Spb1 sequence from strain COH1 and GBS80 sequence from strain 2603V/R.

Polypeptides

The polypeptides, or individual moieties, may, compared to SEQ ID NOs: 2, 3, 9, 10, 13, 21 or 23, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may have one or more (e.g. 1, 2, 3, 4, 5, 6, 7, 8, 9, 10, etc.) single amino acid deletions relative to a reference sequence. The polypeptides may also include one or more (e.g. 1, 2, 3, 4, 5, 6, 7, 8, 9, 10, etc.) insertions (e.g. each of 1, 2, 3, 4 or 5 amino acids) relative to a reference sequence.

The polypeptides can be prepared in many ways, as described above. The polypeptides can also take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.), as described above. The polypeptides are preferably provided in purified or substantially purified form, as described above.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. The polypeptides can be naturally or non-naturally glycosylated (i.e. the polypeptide has a glycosylation pattern that differs from the glycosylation pattern found in the corresponding naturally occurring polypeptide).

The polypeptides may be at least 40 amino acids long (e.g. at least 40, 50, 60, 70, 80, 90, 100, 120, 140, 160, 180, 200, 220, 240, 260, 280, 300, 350, 400, 450, 500 or more). The polypeptides may be shorter than 1100 amino acids.

Pre-Immunisation

In a second aspect, the invention provides a method for immunising a patient against infection by GBS comprising the step of administering to the patient a conjugate that is a capsular saccharide from GBS conjugated to a diphtheria toxoid or derivative thereof, wherein the patient has been pre-immunised with a diphtheria toxoid or derivative thereof. Typically, the conjugate is one of the GBS conjugates in an immunogenic composition of the first aspect of the invention, as described above. In other words, immunogenic compositions of the first aspect of the invention wherein at least one conjugate is a capsular saccharide from GBS conjugated to a diphtheria toxoid or derivative thereof may be used in the second aspect of the invention. The capsular saccharide conjugated to the diphtheria toxoid or derivative thereof in the composition may for example be from GBS serotype Ia, Ib or III. In particular, the capsular saccharide may be from GBS serotype III (as exemplified below). In this aspect it is typical for all of the capsular saccharides from GBS in the composition to be conjugated to a diphtheria toxoid or derivative thereof. Where the carrier or pre-immunisation antigen is a derivative of a diphtheria toxoid then that derivative preferably remains immunologically cross-reactive with Dt, and is preferably CRM197. The inventors have found that conjugates that are capsular saccharides from GBS conjugated to a diphtheria toxoid or derivative thereof do not seem to suffer from carrier-induced epitopic suppression (or “carrier suppression”, as it is generally known), particularly suppression arising from carrier priming As discussed below, “carrier suppression” is the phenomenon whereby pre-immunisation of an animal with a carrier protein prevents it from later eliciting an immune response against a new antigenic epitope that is presented on that carrier [230]. In contrast to this known phenomenon, the inventors have found that the immune response to GBS capsular saccharide-diphtheria toxoid or derivative thereof conjugates may in fact be improved by pre-immunisation with the diphtheria toxoid or derivative thereof.

As reported in reference 231, where several vaccine antigens contain the same protein component (being used as an immunogen and/or as a carrier protein in a conjugate) then there is the potential for interference between those antigens. In reference 231, the immune response against an antigen that was conjugated to a tetanus toxoid (Tt) carrier was suppressed by pre-existing immunity against Tt.

Reference 232 reports how a combination of D-T-P vaccines with a Hib conjugate vaccine was adversely affected where the carrier for the Hib conjugate was the same as the tetanus antigen from the D-T-P vaccine. The authors concludes that this “carrier suppression” phenomenon, arising from interference by a common protein carrier, should be taken into account when introducing vaccines that include multiple conjugates.

In contrast to references 231 and 232, reference 233 reported that priming with tetanus toxoid had no negative impact on the immune response against a subsequently-administered Hib-Tt conjugate, but suppression was seen in patients with maternally acquired anti-Tt antibodies. In reference 234, however, an “epitopic suppression” effect was reported for a Tt-based peptide conjugate in patients having existing anti-Tt antibodies resulting from tetanus vaccination.

In reference 235, it was suggested that a conjugate having CRM197 (a detoxified mutant of diphtheria toxin) as the carrier may be ineffective in children that had not previously received diphtheria toxin as part of a vaccine (e.g. as part of a D-T-P or D-T vaccine). This work was further developed in reference 236, where a carrier priming effect by D-T immunisation was seen to persist for subsequent immunisation with Hib conjugates.

In reference 237, the authors found that pre-immunisation with a diphtheria or tetanus toxoid carrier protein reduced the increase in anti-Hib antibody levels after a subsequent immunisation with the Hib capsular saccharide conjugated to those carriers, with IgG1 and IgG2 being equally affected. Responses to the carrier portions of the conjugates were also suppressed. Furthermore, a more general non-epitope-specific suppression was seen, as pre-immunisation with one conjugate was seen to affect immune responses against both the carrier and saccharide portions of a second conjugate that was administered four weeks later.

The use of different carrier proteins in a single multivalent pneumococcal conjugate vaccine is reported in reference 238, with multiple carriers being used in order to avoid carrier suppression. The authors predict that there is a maximum load of a carrier protein that can be tolerated in a multivalent conjugate vaccine without giving rise to negative interference. In reference 239 it was reported that pneumococcal conjugate vaccines including mixed carrier proteins elicited, in parallel to the anti-pneumococcus response, unintentional booster responses to the carriers.

In reference 240, an investigation of whether diphtheria and tetanus boosters could be administered with monovalent meningococcal serogroup C conjugates, it was found that titres against the meningococcal conjugate were reduced where the carrier was tetanus toxoid carrier and the patient had received prior immunisation with a tetanus-containing vaccine.

In addition to the problem of priming with a carrier having a negative impact on immune responses against saccharide conjugates, the reverse can also occur i.e. immunisation with a conjugate can have a negative impact on immune responses against the carrier [241].

This second aspect of the invention therefore provides a method for immunising a patient against infection by GBS comprising the step of administering to the patient a conjugate that is a capsular saccharide from GBS conjugated to a diphtheria toxoid or derivative thereof, wherein the patient has been pre-immunised with a diphtheria toxoid or derivative thereof. This aspect also provides a conjugate that is a capsular saccharide from GBS conjugated to a diphtheria toxoid or derivative thereof for use in immunising a patient against infection by GBS, wherein the patient has been pre-immunised with a diphtheria toxoid or derivative thereof. This aspect further provides the use of a conjugate that is a capsular saccharide from GBS conjugated to a diphtheria toxoid or derivative thereof in the manufacture of a medicaument for immunising a patient against infection by GBS, wherein the patient has been pre-immunised with a diphtheria toxoid or derivative thereof.

The Pre-Immunised Patient

This patient to be immunised has been pre-immunised with a diphtheria toxoid or derivative thereof. The diphtheria toxoid or derivative thereof may have been administered as the carrier in a conjugate of a capsular saccharide of an organism other than GBS and a diphtheria toxoid or derivative thereof. Typical pre-immunisation will have included: a diphtheria toxoid antigen; a Hib capsular saccharide conjugate using a diphtheria toxoid or CRM197 carrier; and/or a pneumococcal capsular saccharide conjugate using a diphtheria toxoid or CRM197 carrier.

The patient will have received at least one (e.g. 1, 2, 3 or more) dose of the pre-immunisation antigen(s), and that dose (or the earliest of multiple doses) will have been administered to the patient at least six (e.g. 6, 9, 12, 15, 18, 21, 24, 36, 48, 60, 120, 180, 240, 300 or more) months before the immunisation with the GBS conjugates according to this aspect of invention. In a preferred group of patients, the pre-immunisation took place within 3 years of birth e.g. within 2 years of birth, within 1 year of birth, within 6 months of birth, or even within 3 months, 2 months or 1 month of birth. Suitable patients to be immunised according to this aspect of the invention are described above in the section Methods of treatment.

Where the pre-immunisation antigen is a diphtheria toxoid then the patient will typically have received the toxoid as the ‘D’ antigen in a D-T-P or a D-T pre-immunisation. Such immunisations are typically given to newborn children at ages 2, 3, and 4 months. Where the immunisation includes a pertussis vaccine, that vaccine may be a whole cell or cellular pertussis vaccine (‘Pw’), but is preferably an acellular pertussis vaccine (‘Pa’). Pre-immunisation Pa vaccines will generally include one, two or three of the following well-known and well-characterised B.pertussis antigens: (1) pertussis toxoid (‘PT’), detoxified either by chemical means or by site-directed mutagenesis e.g. the ‘9K/129G’ mutant [242]; (2) filamentous haemagglutinin (‘FHA’); (3) pertactin (also known as ‘69 kiloDalton outer membrane protein’).

Acellular pertussis vaccines may also include agglutinogen 2 and/or agglutinogen 3. The ‘T’ antigen in a D-T-P pre-immunisation is typically a tetanus toxoid.

Where the pre-immunisation antigen is a diphtheria toxoid then the patient may also or alternatively have received the toxoid as the carrier protein of a protein-saccharide conjugate. Such conjugates include the ‘PRP-D’ Hib conjugate [see Table 14-7 of ref. [243] e.g. the PROHIBIT™ product.

Where the pre-immunisation antigen is CRM197 then the patient will typically have been pre-immunised with a Hib conjugate and/or a multivalent pneumococcal conjugate. Such immunisations are typically given to newborn children at ages 2, 3, and 4 months. Hib conjugates that use a CRM197 carrier include the ‘HbOC’ conjugates [Table 14-7 of ref.243] e.g. the HIBTITER™ product. Pneumococcal conjugates that use a CRM197 carrier include the 7-valent PCV7 mixtures e.g. the PREVNAR™ vaccine [244]. The patient may also have been pre-immunised with a serogroup C meningococcal (‘MenC’) conjugate. MenC conjugates that use CRM197 carrier include MENINVACT™/MENJUGATE™ [245] and MENINGITEC™.

Where pre-immunisation was with a conjugated antigen then the patient will almost inevitably have also received a small amount of free diphtheria toxoid (or derivative) as a result of low-level contamination of the conjugate (e.g. caused by hydrolysis of the conjugate during storage), but this small amount will not typically have been adequate to provide a significant immune response.

Diphtheria toxoid is a well known and well characterised protein [e.g. see chapter 13 of ref. Error! Bookmark not defined.] that can be obtained by treating the ADP-ribosylating exotoxin of Corynebacterium diphtheriae with an inactivating chemical, such as formalin or formaldehyde. CRM197 is also well known and well characterised [246-249], and has been widely used as a carrier in conjugated saccharide vaccines. CRM197 and Dt share many carrier epitopes.

The result of the pre-immunisation is that the patient's immune system has been exposed to the pre-immunisation antigens. For pre-immunisation with diphtheria toxoid (Dt), this generally means that the patient will have raised an anti-Dt antibody response (typically to give an anti-Dt titer>0.01 IU/ml) and will possess memory B and/or T lymphocytes specific for Dt i.e. pre-immunisation with Dt is typically adequate to elicit an anamnestic anti-Dt immune response in the patient. For pre-immunisation where Dt (or derivative) is a carrier for a saccharide within a conjugate then the pre-immunisation will have raised an anti-saccharide response and the patient will possess memory B and/or T lymphocytes specific for the saccharide i.e. the pre-immunisation is typically adequate to elicit an anamnestic anti-saccharide immune response in the patient. The pre-immunisation was preferably adequate to elicit protective immunity in the patient e.g. against diphtheria disease.

Thus the patients to be immunised according to this aspect of the invention are distinct from patients in general, as they are members of a subset of the general population whose immune systems have already mounted an immune response to the pre-immunisation antigens, such that immunisation according to this aspect with a GBS conjugate that includes a diphtheria toxoid (or derivative thereof) carrier elicits a different immune response in the subset than in patients who have not previously mounted an immune response to the pre-immunisation antigens. Patients who have been pre-immunised with Dt (or derivative) as the carrier of a conjugate (particularly of a Hib conjugate) are preferred. Particularly preferred patients have been pre-immunised with Dt (or derivative) as the carrier of a conjugate and also with Dt as an unconjugated immunogen.

As well as having been pre-immunised with a diphtheria toxoid (or derivative), in conjugated or non-conjugated form, the patient may have been pre-immunised with other antigens. Such antigens include, but are not limited to: pertussis antigen(s)—see above; tetanus toxoid—see above; Haemophilus influenzae type B—see above; hepatitis B surface antigen (HBsAg); poliovirus, such as an inactivated poliovirus vaccine (IPV); Streptococcus pneumoniae—see above; influenza virus; BCG; hepatitis A virus antigens; measles virus; mumps virus; rubella virus; varicella virus; etc.

The patient may or may not have been pre-immunised with one or more GBS conjugate(s). In some preferred embodiments, at the time when a patient first receives a GBS conjugate, they have already been pre-immunised with Dt (or derivative). In other embodiments, a GBS conjugate is administered to a patient who has already been pre-immunised with both (i) Dt or a derivative and (ii) a GBS conjugate.

Tetanus Toxoid Carriers

Although this second aspect of the invention has been described above in relation to diphtheria toxoid carriers (and their derivatives), with tetanus toxoid preferably not being used, alternative embodiments of this aspect use a tetanus toxoid (or derivative) carrier, with diphtheria toxoid preferably not being used. In these alternative embodiments then the above definitions can be modified accordingly.

For instance, the second aspect of the invention provides a method for immunising a patient against infection by GBS comprising the step of administering to the patient a conjugate that is a capsular saccharide from GBS conjugated to a tetanus toxoid or derivative thereof, wherein the patient has been pre-immunised with a tetanus toxoid or derivative thereof. This aspect also provides a conjugate that is a capsular saccharide from GBS conjugated to a tetanus toxoid or derivative thereof for use in immunising a patient against infection by GBS, wherein the patient has been pre-immunised with a tetanus toxoid or derivative thereof. This aspect further provides the use of a conjugate that is a capsular saccharide from GBS conjugated to a tetanus toxoid or derivative thereof in the manufacture of a medicaument for immunising a patient against infection by GBS, wherein the patient has been pre-immunised with a tetanus toxoid or derivative thereof. Conjugates that are capsular saccharides from GBS conjugated to a tetanus toxoid or derivative thereof may not suffer from carrier suppression, particularly suppression arising from carrier priming. The immune response to GBS capsular saccharide-tetanus toxoid or derivative thereof conjugates may in fact be improved by pre-immunisation with the tetanus toxoid or derivative thereof.

Typically, the conjugate is one of the GBS conjugates in an immunogenic composition of the first aspect of the invention, as described above. In other words, immunogenic compositions of the first aspect of the invention wherein at least one conjugate is a capsular saccharide from GBS conjugated to a tetanus toxoid or derivative thereof may be used in the second aspect of the invention. The capsular saccharide conjugated to the tetanus toxoid or derivative thereof in the composition may for example be from GBS serotype Ia, Ib or III. In particular, the capsular saccharide may be from GBS serotype III. In this aspect it is typical for all of the capsular saccharides from GBS in the composition to be conjugated to a tetanus toxoid or derivative thereof.

Tetanus toxoid is a well known protein [e.g. see chapter 27 of ref 243], and can be obtained by inactivating the ADP-ribosylating exotoxin of Clostridium tetani. Patients will typically have received tetanus toxoid as the ‘T’ antigen in a D-T-P or a D-T pre-immunisation, or as the carrier protein in a conjugate. Such conjugates include the ‘PRP-T’ Hib conjugate [see Table 14-7 of ref 243] e.g. the ACTIHIB™, OMNIHIB™ and HIBERIX™ products.

General

The term “comprising” encompasses “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

It will be appreciated that sugar rings can exist in open and closed form and that, whilst closed forms are shown in structural formulae herein, open forms are also encompassed by the invention. Similarly, it will be appreciated that sugars can exist in pyranose and furanose forms and that, whilst pyranose forms are shown in structural formulae herein, furanose forms are also encompassed. Different anomeric forms of sugars are also encompassed.

Unless specifically stated, a process comprising a step of mixing two or more components does not require any specific order of mixing. Thus components can be mixed in any order. Where there are three components then two components can be combined with each other, and then the combination may be combined with the third component, etc.

Antibodies will generally be specific for their target. Thus they will have a higher affinity for the target than for an irrelevant control protein, such as bovine serum albumin.

Unless otherwise stated, identity between polypeptide sequences is preferably determined by the Smith-Waterman homology search algorithm as implemented in the MPSRCH program (Oxford Molecular), using an affine gap search with parameters gap open penalty=12 and gap extension penalty=1.

### MODES FOR CARRYING OUT THE INVENTION

Conjugate Production

Purified capsular saccharides from Streptococcus agalactiae serotypes Ia, Ib and III were conjugated to a carrier protein by periodate oxidation followed by reductive amination (ref. Error! Bookmark not defined.). Purified, desialylated capsular saccharide from Streptococcus agalactiae serotype V was conjugated to a carrier protein by periodate oxidation followed by reductive amination (ref 14). The carrier protein in most cases was CRM197. Tetanus toxoid was used as a carrier protein where specifically indicated.

Mouse Study (1).

In this study, the effect of the adjuvant on the efficacy and immunogenicity of GBS serotype Ia, Ib and III conjugates, either as monovalent or combination vaccines, was evaluated in an active maternal-neonatal challenge mouse model.

The maternal-neonatal challenge mouse model, adapted from the reference 250, is used to to assess the efficacy in neonates of specific antibodies acquired transplacentally from actively vaccinated dams. Specifically, female CD-1 mice, aged between 5-6 weeks from Charles River Laboratories (Calco, Italy), are vaccinated by intra-peritoneal injection with two or three immunizations on days 1, 21 and eventually 35, with or without adjuvant. After the last immunization, mice are bred and kept until delivery. An inoculum of a GBS strain (0.05 mL of Todd-Hewitt broth), lethal for 90% of non-immunized pups (100-1000 fold LD50), is used to challenge the neonatal mice. Challenge is by the intra-peritoneal route within 48 hours of birth. The number of surviving pups at 72 hours is recorded and survival rates are compared in all treated groups using the Fisher's exact test. In a control group, dams receive PBS by the same route and using the same dosing schedule. Two weeks after the last immunization, blood samples are collected for immunogenicity assessment using two in vitro assays (the ELISA and Opsonophagocytosis assays described below).

The ELISA assay is performed to determine the titer of GBS-specific antibodies produced following immunization. ELISA is also used to quantify the total IgG against each capsular saccharde antigen. Serum from each individual mouse is analyzed and the Geometric Mean Titer (GMT) calculated for each group. Antibody titers for capsular saccharde types Ia, Ib and III are expressed as Mouse ELISA Unit (MEU) and are calculated based on the Reference Line Assay Method.

The Opsonophagocytosis assay (OPA) is performed to evaluate the titer of vaccine-induced antibodies capable of complement-mediated GBS killing (using the approach described in reference 1). The assay is performed by combining the following components: bacteria, phagocytic cells (PMNs extracted from human blood or the differentiated HL-60 cell line), complement and immune sera. Aliquots of the reaction mix are plated before and after a 1 h incubation at 37° C. to determine the remaining colony forming units (CFU). The amount of opsonophagocytic killing (log kill) is determined by subtracting the log of the surviving colony number from the log of the CFU number present at the initial time-point. A pre-immune serum, and heat-inactivated complement without PMNs, is used as negative control. Bactericidal titer is expressed as reciprocal serum dilution leading to reduction in 50% of bacteria.

In this study, female CD1 mice were immunized with two doses (1 μg each) of the three different conjugates in the presence of adjuvant (aluminum hydroxide or MF59) on days 0 and 21. The neonates were challenged with type specific strains as shown below in Table 1.

TABLE 1
Determination of the protection level and antibody titers
obtained with GBS serotype Ia, Ib and III/CRM197 conjugates
in the presence of aluminum hydroxide or MF59 in the active
maternal-neonatal challenge mouse model.

| | | GMT | Challenge | Alive/ | Survival |
|---|---|---|---|---|---|
| Antigen | Adjuvant | titers | Strain (type) | Treated | (%) |
| CRM-Ia | Al—H | 281 | A909 (Ia) | 77/80 | 96 |
| CRM-Ia | MF59 | 1253 | A909 (Ia) | 66/75 | 88 |
| PBS | — | — | A909 (Ia) | 1/65 | 1 |
| CRM-Ib | Al—H | 1097 | 7357B (Ib) | 65/70 | 93 |
| CRM-Ib | MF59 | 7843 | 7357B (Ib) | 47/60 | 78 |
| PBS | — | — | 7357B (Ib) | 6/70 | 8 |
| CRM-III | Al—H | 234 | COH1 (III) | 44/45 | 98 |
| CRM-III | MF59 | 898 | COH1 (III) | 68/80 | 85 |
| PBS | — | — | COH1 (III) | 0/68 | 0 |

— Not applicable

High levels of protection were achieved with the monovalent vaccines for all three serotypes with both aluminum hydroxide and MF59. However, slightly lower survival rates were obtained with the adjuvant MF59 even in presence of higher antibody titers.

In an additional experiment, mice were immunized with three doses of combinations at 1 μg of each conjugate in the presence of adjuvant on days 0, 21 and 35. The neonates were challenged with type specific strains as shown below in Table 2.

TABLE 2
Determination of the protection level and antibody titers obtained with
combinations of GBS serotype Ia, Ib and III/CRM197 conjugates in the
presence or absence of adjuvant in the active maternal-neonatal
challenge mouse model.

| | | | Challenge | Alive/ | Survival |
|---|---|---|---|---|---|
| Antigen | Adjuvant | GMT titers | Strain (type) | Treated | (%) |
| Combo | Al—H | 1279 | 090 (Ia) | 27/30 | 90 |
| Combo | MF59 | 4592 | 090 (Ia) | 61/65 | 94 |
| Combo | No-Adjuvant | 218 | 090 (Ia) | 59/77 | 77 |
| PBS | — | — | 090 (Ia) | 0/70 | 0 |
| Combo | Al—H | 2086 | H36B (Ib) | 64/70 | 91 |
| Combo | MF59 | 5921 | H36B (Ib) | 65/80 | 81 |
| Combo | No-Adjuvant | 386 | H36B (Ib) | 56/70 | 80 |
| PBS | — | — | H36B (Ib) | 6/79 | 7 |
| Combo | Al—H | 596 | M781 (III) | 30/40 | 75 |
| Combo | MF59 | 1978 | M781 (III) | 70/70 | 100 |
| Combo | No-Adjuvant | 163 | M781 (III) | 60/79 | 76 |
| PBS | — | — | M781 (III) | 3/77 | 4 |

Combo = CRM197-Ia + CRM197-Ib + CRM197-III;
—: Not applicable

High levels of protection were achieved with the combination vaccines in all three formulations in the presence or absence of adjuvant, although lower antibody titers were achieved in the absence of adjuvant.

Mouse Study (2)

In this study, the effect of lyophilization on the efficacy and immunogenicity of the serotype Ia, Ib and III/CRM197 conjugates in the active maternal-neonatal challenge mouse model was evaluated. Mice were immunized with two doses (1 μg each) of the three different conjugates, in the presence or absence of adjuvant on days 0, and 21. The neonates were challenged with type specific strains as shown below in Table 3.

TABLE 3
Determination of the protection level, antibody titers and bactericidal titers
achieved by the serotype Ia, Ib and III/CRM197 conjugates, when administered to mice as
liquid or lyophilized antigen in the presence or absence of aluminum hydroxide in the active
maternal-neonatal challenge mouse model.

| | | | Bactericidal | Challenge | Alive/ | Survival |
|---|---|---|---|---|---|---|
| Antigen | Adjuvant | GMT titer | titer | Strain (type) | Treated | (%) |
| CRM-Ia | PBS | 48 | <100 | 090 (Ia) | 46/78 | 59 |
| Lyophilized | | | | | | |
| CRM-Ia | Al—H | 1201 | 567 | 090 (Ia) | 47/51 | 92 |
| Lyophilized | | | | | | |
| CRM-Ia Liquid | PBS | 14 | <100 | 090 (Ia) | 11/60 | 18 |
| CRM-Ia Liquid | Al—H | 901 | 436 | 090 (Ia) | 58/60 | 96 |
| PBS | Al—H | — | — | 090 (Ia) | 1/57 | 2 |
| CRM-Ib | PBS | 23 | 366 | H36B (Ib) | ND | ND |
| Lyophilized | | | | | | |
| CRM-Ib | Al—H | 172 | 2146 | H36B (Ib) | ND | ND |
| Lyophilized | | | | | | |
| CRM-Ib Liquid | PBS | 27 | 375 | H36B (Ib) | 12/40 | 30 |
| CRM-Ib Liquid | Al—H | 169 | 1756 | H36B (Ib) | 73/78 | 93 |
| PBS | Al—H | — | — | H36B (Ib) | 6/72 | 8 |
| CRM-III | PBS | 59 | 419 | M781 (III) | ND | ND |
| Lyophilized | | | | | | |
| CRM-III | Al—H | 429 | 1861 | M781 (III) | ND | ND |
| Lyophilized | | | | | | |
| CRM-III Liquid | PBS | 127 | 1707 | M781 (III) | 48/50 | 96 |
| CRM-III Liquid | Al—H | 198 | 1100 | M781 (III) | 44/45 | 98 |
| PBS | Al—H | — | — | M781 (III) | 5/66 | 7 |

— Not applicable;
ND: not determined

The lyophilization process did not affect the immunogenicity of the GBS conjugates. Antibody titers and bactericidal titers were comparable in mice that received both liquid and lyophilized formulations.

Mouse Study (3)

In this study, the effect of lyophilization on the efficacy of the serotype V/CRM197 conjugate in the active maternal-neonatal challenge mouse model was evaluated. Mice were immunized with two doses (1, 5 or 10 μg each) of the conjugates in the presence or absence of adjuvant on days 0 and 21. The neonates were challenged with a type V strain. The results are shown in Table 4 below.

TABLE 4
Determination of the protection level achieved by the serotype V/CRM197
conjugate, when administered to mice as liquid or lyophilized antigen in
presence or absence of aluminum hydroxide in the active maternal-
neonatal challenge mouse model.

| | No adjuvant | Aluminum hydroxide |
|---|---|---|
| Antigen | Dead/treated (% survival) | |
| CRM-V Lyophilized (1 μg) | 20/30 (33) | 13/44 (70) |
| CRM-V Lyophilized (5 μg) | 28/39 (28) | 31/40 (22) |
| CRM-V Lyophilized (10 μg) | 40/54 (26) | 33/49 (33) |
| CRM-V Liquid (1 μg) | 63/70 (10) | 19/47 (59) |
| CRM-V Liquid (5 μg) | 29/40 (27) | 37/60 (38) |
| CRM-V Liquid (10 μg) | 46/52 (11) | 46/70 (34) |
| Placebo Lyophilized | 108/119 (9) | 70/88 (20) |

The lyophilization process did not affect the immunogenicity of the GBS conjugate. Survial rates were comparable in mice that received both liquid and lyophilized formulations.

Mouse Study (4)

In this study, the effect of different doses on the efficacy of a mixture of the GBS serotype Ia, Ib, III and V conjugates was evaluated in the active maternal-neonatal challenge mouse model. Mice were immunized with two doses of the combination at 0.2, 1 or 5 μg of each of the GBS serotype Ia, Ib, III and V conjugates without adjuvant on days 0 and 21. The neonates were challenged with type specific strains as shown below in Table 5 below.

TABLE 5
Determination of the protection level obtained with combinations of GBS
serotype Ia, Ib, III and V/CRM197 conjugates in the active maternal-
neonatal challenge mouse model.

| | | Challenge Strain (type) | | | |
|---|---|---|---|---|---|
| | | | | | CJB111 |
| | | 090 (Ia) | H36B (Ib) | M781 (III) | (V) |
| Antigen | Doses | Dead/treated (% survival) | | | |
| Combo | 5 μg each | 17/60 (72) | 13/70 (81) | 18/70 (74) | 20/68 (70) |
| Combo | 1 μg each | 23/70 (67) | 18/70 (74) | 6/60 (90) | 44/66 (33) |
| Combo | 0.2 μg each | 14/51 (72) | 25/79 (68) | 18/78 (77) | 41/60 (32) |
| PBS | 0 | 57/58 (2) | 49/50 (2) | 49/50 (2) | 45/50 (10) |

Combo = CRM197-Ia + CRM197-Ib + CRM197-III + CRM197-V
Higher dosage of GBS serotype V conjugate raised the level of protection.

Mouse Study (5)

In this study, the effect of different numbers of doses on the efficacy of a mixture of the GBS serotype Ia, Ib, III and V conjugates was evaluated in the active maternal-neonatal challenge mouse model. Mice were immunized with one, two or three doses (1 μg of each conjugate) of the combination in the presence of an alum adjuvant on days 0, 21 and 35 as appropriate. The neonates were challenged with type specific strains as shown below in Table 6 below.

TABLE 6
Determination of the protection level obtained with combinations of GBS
serotype Ia, Ib, III and V/CRM197 conjugates in the presence of adjuvant
in the active maternal-neonatal challenge mouse model.

| | | Challenge Strain (type) | | | |
|---|---|---|---|---|---|
| | | 090 (Ia) | H36B (Ib) | M781 (III) | CJB111 (V) |
| Antigen | Doses | Dead/treated (% survival) | | | |
| Combo | 3 | 5/80 (94) | 4/60 (93) | 1/50 (98) | 32/70 (54) |
| Combo | 2 | 6/80 (92) | 11/60 (82) | 1/70 (98) | 42/57 (26) |
| Combo | 1 | 61/90 (32) | 21/50 (58) | 4/60 (93) | 52/58 (10) |
| PBS | 3 | 50/50 (0) | 49/50 (2) | 52/58 (10) | 59/60 (2) |

Combo = CRM197-Ia + CRM197-Ib + CRM197-III + CRM197-V

Bactericidial titers were measured following administration of the mixture of serotype Ia, Ib, III and V conjugates in this study. OPA titers are shown below:

| | Ia | Ib | III | V |
|---|---|---|---|---|
| Post-3 | 515 | >900 | 1174 | 135 |
| Post-2 | <100 | 455 | 525 | <100 |
| Post-1 | <100 | 182 | 358 | <100 |

The number of immunizations strongly affected the immune response to the GBS serotype V conjugate.

Mouse Study (6)

In this study, the efficacy of a mixture of the GBS serotype Ia, Ib, III and V conjugates compared to the GBS serotype V conjugate alone was evaluated in the active maternal-neonatal challenge mouse model. Mice were immunized with three doses of combinations at 1 μg of each conjugate or GBS serotype V conjugate at 1 μg in the presence of alum adjuvant on days 0, 21 and 35. The neonates were challenged with the CJB111 and 2603 V/R type V strains. The results are shown in Table 7 below.

TABLE 7
Determination of the protection level obtained with combinations of GBS
serotype Ia, Ib, III and V/CRM197 conjugates or GBS serotype V
conjugate alone in the presence of adjuvant in the active maternal-
neonatal challenge mouse model.

Challenge Strain (type)

| | CJB111 (V) | 2603 V/R (V) |
|---|---|---|
| Antigen | Dead/treated | (% survival) |
| CRM197-V | 78/253 (69) | 9/117 (92) |
| Combo | 218/583 (63) | 32/118 (73) |
| PBS | 333/350 (5) | 138/149 (6) |

Combo = CRM197-Ia + CRM197-Ib + CRM197-III + CRM197-V

The immune response to the capsular saccharide from GBS serotype V was diminished when GBS serotype la, Ib and III conjugates were also present in the composition.

Mouse Study (7)

In this study, the effect of adjuvant on the immunogenicity and efficacy of a mixture of the GBS serotype Ia, Ib, III and V conjugates compared to the GBS serotype V conjugate alone was evaluated in the active maternal-neonatal challenge mouse model. Mice were immunized with three doses of combinations at 1 μg of each conjugate or GBS serotype V conjugate at 1 μg in the presence or absence of adjuvant on days 0, 21 and 35. The neonates were challenged with the CJB111 type V strain. The results are shown in Table 8 below.

TABLE 8
Determination of the protection level, antibody titers and bactericidal titers
obtained with combinations of GBS serotype Ia, Ib, III and V/CRM197
conjugates or GBS serotype V conjugate alone in the presence or
absence of adjuvant in the active maternal-neonatal challenge
mouse model.

| | | | | Dead/treated |
|---|---|---|---|---|
| Antigen | Adjuvant | GMT titer | Bactericidal titer | (% survival) |
| CRM197-V | PBS | 83 | 838 | 21/68 (69) |
| Combo | PBS | 22 | 251 | 62/130 (52) |
| CRM197-V | Alum | 130 | 1430 | 30/80 (62) |
| Combo | Alum | 59 | <100 | 66/148 (55) |
| PBS | Alum | — | — | 122/131 (7) |

Combo = CRM197-Ia + CRM197-Ib + CRM197-III + CRM197-V;
—Not applicable

Once again, the immune response to the capsular saccharide from GBS serotype V was diminished when GBS serotype la, Ib and III conjugates were also present in the composition. Survival was improved by addition of adjuvant, even though addition of adjuvant to the GBS serotype V conjugate alone did not have this effect in this experiment.

Mouse Study (8)

In this study, the effect of increasing the dose of GBS serotype V conjugate on the efficacy of a mixture of the GBS serotype Ia, Ib, III and V conjugates was evaluated in the active maternal-neonatal challenge mouse model. Mice were immunized with two doses of combinations at 1 μg of each conjugate or two doses of combinations at 1 μg of the the GBS serotype Ia, Ib, III conjugates and 5 μg of the GBS serotype V conjugate in the presence or absence of adjuvant on days 0 and 21. The neonates were challenged with type specific strains as shown below in Table 9 below.

TABLE 9
Determination of the protection level obtained with combinations of GBS
serotype Ia, Ib, III and V/CRM197 conjugates in the presence of adjuvant
at with different doses of GBS serotype V conjugate in the active
maternal-neonatal challenge mouse model.

| | | Challenge Strain (type) | | | |
|---|---|---|---|---|---|
| | | 090 (Ia) | H36B (Ib) | M781 (III) | CJB111 (V) |
| Antigen | Adjuvant | Dead/treated (% survival) | | | |
| Combo | PBS | 20/59 (66) | 12/50 (76) | 1/40 (97) | 49/50 (2) |
| Combo | Alum | 36/50 (28) | 10/30 (67) | 1/40 (97) | 22/40 (45) |
| Combo | PBS | 40/40 (0) | 23/26 (11) | 37/40 (7) | 31/38 (18) |
| plus | | | | | |
| Combo | Alum | 13/45 (71) | 15/40 (62) | 0/50 (100) | 26/50 (48) |
| plus | | | | | |

Combo = CRM197-Ia + CRM197-Ib + CRM197-III + CRM197-V (all at 1 μg)
Combo plus = CRM197-Ia + CRM197-Ib + CRM197-III + CRM197-V (all at 1 μg, except for CRM197-V at 5 μg)

In this experiment, the immune response to the capsular saccharide from GBS serotype V in the mixture was once again improved by addition of adjuvant. The response was also improved by increasing the dose of this capsular saccharide in the composition. However, the presence of a high dose of capsular saccharide from GBS serotype V seemed to reduce the response to the capsular saccharides from GBS serotype Ia, Ib and III. This consequence was reduced by addition of adjuvant.

Mouse Study (9)

In this study, the efficacy of a mixture of the GBS serotype Ia, Ib and III conjugates with GBS67 and GBS80 proteins was evaluated in the active maternal-neonatal challenge mouse model. Mice were immunized with combinations in the presence or absence of various different adjuvants. The neonates were challenged with type specific strains as shown below in Table 10 below.

TABLE 10
Determination of the protection level obtained with combinations of GBS serotype
Ia, Ib and III/CRM197 conjugates and GBS67 and GBS80 proteins in the active maternal-
neonatal challenge mouse model.

| | | Challenge Strain (type) | | | | | |
|---|---|---|---|---|---|---|---|
| | | | | | | | JM9130013 |
| | | 090 (Ia) | H36B (Ib) | 3050 (II) | M781 (III) | CJB111(V) | (VIII) |
| Ag | Adjuvant | Dead/treated (% survival) | | | | | |
| Combo | Alum | 9/60 (85) | 6/60 (90) | 12/58 (79) | 0/60 (100) | 11/55 (80) | 28/56 (50) |
| | hydroxide/ | | | | | | |
| | saline | | | | | | |
| Combo | Alum | 19/78 (76) | 5/57 (91) | 16/66 (76) | 4/53 (92) | | 55/80 (31) |
| | hydroxide/ | | | | | | |
| | PBS | | | | | | |
| Combo | MF59 | 4/60 (93) | 12/57 (79) | 18/60 (70) | 3/77 (96) | | 45/70 (36) |
| Combo | None | 13/80 (84) | 11/70 (84) | 28/60 (53) | 14/77 (82) | | 47/59 (20) |
| PBS | Alum | 60/60 (0) | 74/77 (4) | 36/56 (36) | 73/80 (9) | 86/99 (13) | 53/69 (23) |
| | hydroxide/ | | | | | | |
| | saline | | | | | | |
| PBS | None | 70/70 (0) | 73/79 (7) | 42/54 (22) | 74/77 (4) | | 63/74 (15) |

Combo = CRM197-Ia + CRM197-Ib + CRM197-III + GBS67 + GBS80

Antibody titers were measured following administration of the mixture of serotype la, Ib, III and V conjugates and GBS67 and GBS80 proteins in this study. Results from five separate experiments are shown below:

| | Aluminium | Aluminium | | |
|---|---|---|---|---|
| | Hydroxide/saline | Hydroxide/PBS | MF 59 | No Adjuvant |
| GBS 80 | 45395 | 50277 | 15626 | 3358 |
| GBS 67 | 25846 | 29513 | 9616 | 4232 |
| Ps Ia | 811 | 711 | 2190 | 404 |
| Ps Ib | 1929 | 1277 | 2571 | 691 |
| Ps III | 862 | 1043 | 1314 | 275 |

Mouse Study (10)

In this study, the efficacy of a mixture of the GBS serotype Ia, Ib and III conjugates with GBS67 and GBS80 proteins was evaluated in the active maternal-neonatal challenge mouse model. Mice were immunized with three doses of combinations in the presence or absence of various different adjuvants at days 0, 21 and 35. The neonates were challenged with type specific strains as shown below in Table 11 below.

TABLE 11
Determination of the protection level obtained with combinations of GBS
serotype Ia, Ib and III/CRM197 conjugates and GBS67 and GBS80
proteins in the active maternal-neonatal challenge mouse model.

Challenge Strain (type)

| | |
|---|---|
| 090 (Ia) | M781 (III) |

| | | |
|---|---|---|
| Antigen | Adjuvant | Dead/treated (% survival) |

| | | | |
|---|---|---|---|
| Combo | Alum | 4/48 (92) | 6/50 (88) |
| Combo | Alum + CpG | 6/78 (92) | 7/80 (91) |
| Combo | MF59 | 4/69 (94) | 13/55 (76) |
| Combo | MF59 + CpG | 5/66 (92) | 6/70 (91) |
| Combo | PBS | 22/60 (63) | 13/55 (76) |
| Combo | PBS + CpG | 5/59 (91) | 11/66 (83) |
| PBS | — | 69/69 (0) | 60/65 (7) |

Combo = CRM197-Ia + CRM197-Ib + CRM197-III + GBS67 + GBS80

Mouse Study (11)

In this study, the efficacy of a mixture of the GBS serotype Ia, Ib, III and V conjugates with GBS67 protein and a SpbI-GBS80 fusion protein was evaluated in the active maternal-neonatal challenge mouse model. Mice were immunized with the combination in the presence of adjuvant. The neonates were challenged with type specific strains as shown below in Table 12 below.

TABLE 12
Determination of the protection level obtained with combinations of GBS serotype
Ia, Ib, III and V/CRM197 conjugates, GBS67 protein and a SpbI-GBS80 fusion protein
protein in the active maternal-neonatal challenge mouse model.

| | | Challenge Strain (type) | | | | | | | |
|---|---|---|---|---|---|---|---|---|---|
| | | 090 (Ia) | H36B (Ib) | 3050 (II) | COH1 (III) | M781 (III) | M732 (III) | CJB111(V) | JM913 (VII) |
| Ag | Adjvnt | Dead/treated (% survival) | | | | | | | |
| Combo | Alum | 16/70 (77) | 10/70 (86) | 7/70 (90) | 4/77 (95) | 0/60 (100) | 19/30 (36) | 20/58 (65) | 29/50 (42) |
| PBS | Alum | 68/68 (0) | 37/50 (26) | 25/40 (37) | 65/69 (6) | 37/40 (8) | 32/60 (47) | 36/40 (10) | 33/40 (17) |

Combo = CRM197-Ia + CRM197-Ib + CRM197-III + GBS67 protein + SpbI-GBS80 fusion protein

Mouse Study (12)

In this study, the efficacy of GBS serotype Ia, Ib, III and V conjugates was evaluated in the active maternal-neonatal challenge mouse model. Mice were immunized at 1μg of each conjugate. The neonates were challenged with type specific strains as shown below in Table 13 below. The experiment was repeated twice.

TABLE 13
Determination of the protection level obtained with GBS serotype Ia, Ib,
III and V/CRM197 conjugates in the active maternal-neonatal challenge
mouse model.

| | | Challenge Strain (type) | | | |
|---|---|---|---|---|---|
| | | 090 (Ia) | H36B (Ib) | M781 (III) | CJB111 (V) |
| Expt | Antigen | Dead/treated (% survival) | | | |
| 1 | CRM197- | 10/70 (86) | 64/70 (9) | 76/80 (5) | |
| | Ia | | | | |
| | CRM197- | 48/94 (51) | 12/99 (88) | | |
| | Ib | | | | |
| | CRM197- | 69/70 (1) | | 3/60 (95) | 60/68 (12) |
| | III | | | | |
| | CRM197- | | | 80/89 (10) | 5/100 (95) |
| | V | | | | |
| | PBS/Alum | 50/50 (0) | 39/40 (2) | 61/69 (12) | 10/10 (0) |
| 2 | CRM197- | 14/78 (82) | 68/70 (2) | 41/42 (2) | |
| | Ia | | | | |
| | CRM197- | 66/110 (40) | 2/110 (98) | | |
| | Ib | | | | |
| | CRM197- | 70/80 (12) | | 1/60 (98) | |
| | III | | | | |
| | CRM197- | | | | 38/192 (80) |
| | V | | | | |
| | PBS/Alum | 45/45 (0) | 57/58 (2) | 32/36 (11) | 50/58 (14) |

The conjugate comprising capsular saccharide from GBS serotype Ib conferred protection against GBS serotype Ia in addition to GBS serotype Ib.

Mouse Study (13)

In this study, capsular saccharide conjugated to either tetanus toxoid (TT) carrier protein or CRM197 carrier protein were tested and compared for their immunogenicity. Female CD1 mice were immunized with two doses of 1 μg each of GBS serotype Ia, Ib and III conjugates with aluminum hydroxide adjuvant on days 0 and 21. The neonates were challenged with specific strain types as shown below in Table 14.

TABLE 14
Determination of the protection achieved by GBS serotype Ia, Ib and
III capsular saccharide conjugated to TT or CRM in the active maternal-
neonatal challenge mouse model.

| | | | Carrier | |
|---|---|---|---|---|
| | Challenge strain | Carrier protein | protein | |
| CPS type | (type) | Tetanus Toxoid* | Crm* | PBS* |
| Ia | 090 (Ia) | 78 (52/67) | 86 (54/63) | 0 (0/59) |
| Ib | 7357B (Ib) | 62 (50/80) | 73 (71/97) | 0 (0/38) |
| III | COH1 (III) | 97 (37/38) | 93 (95/102) | 2 (1/48) |

*% Survival (Alive/treated)

The survival rates in the groups immunized with CRM197 conjugates of all three serotypes were comparable to the survival rates observed in the immunized group with TT conjugates. Based on these results, CRM197 was selected as the carrier protein for further development.

Mouse Study (14)

In this study, the impact of the level of capsular saccharide oxidation during the covalent conjugation process on immunogenicity was evaluated. Several batches of GBS serotype Ia, Ib and III conjugates were obtained with the saccharides prepared at different percentages of oxidation conjugated either to TT and/or to CRM197 and tested in female CD1 mice for their immunogenicity. Mice were immunized with two doses (1 μg each) of the three different conjugates in the presence of aluminum hydroxide adjuvant on days 0 and 21. The neonates were challenged with type specific strains as shown below in Table 15.

TABLE 15
Determination of the protection level, antibody titers and bactericidal titers of GBS
serotype Ia and III capsular saccharide conjugated either to TT or CRM197 and different
percentage of oxidation using the active maternal-neonatal challenge mouse model.

| | Oxidation | | Challenge | | Survival | Bactericidal |
|---|---|---|---|---|---|---|
| Antigen | level (%) | GMT titers | Strain (type) | Alive/Treated | (%) | Titer |
| CRM-Ia | 5.1 | ND | A909 (Ia) | 42/80 | 52 | ND |
| CRM-Ia | 14.2 | ND | A909 (Ia) | 58/60 | 97 | ND |
| CRM-Ia | 44.7 | ND | A909 (Ia) | 48/78 | 61 | ND |
| CRM-Ia | 79 | ND | A909 (Ia) | 6/50 | 12 | ND |
| PBS | — | — | A909 (Ia) | 11/87 | 13 | — |
| TT-III | 3.9 | 5135 | COH1 (III) | 66/78 | 85 | 575 |
| TT-III | 16 | 7662 | COH1 (III) | 55/59 | 93 | 470 |
| TT-III | 20 | 6850 | COH1 (III) | 47/48 | 98 | 1320 |
| TT-III | 55 | 13290 | COH1 (III) | 64/70 | 91 | 1320 |
| PBS | — | — | COH1 (III) | 1/79 | 1 | — |
| CRM-III | 4.3 | 972 | COH1 (III) | 64/100 | 64 | 127 |
| CRM-III | 17.5 | 812 | COH1 (III) | 77/83 | 93 | 150 |
| CRM-III | 40.9 | 2484 | COH1 (III) | 98/107 | 91 | 183 |
| CRM-III | 61.8 | 8690 | COH1 (III) | 75/85 | 88 | 140 |
| CRM-III | 78.9 | 58629 | COH1 (III) | 67/80 | 84 | 150 |
| PBS | — | — | COH1 (III) | 0/73 | 0 | — |

— not applicable

The survival rates reached a peak when the dams were immunized with conjugates prepared from saccharides oxidized at 15-20%. The antibody titers increased with increasing levels of oxidation without any impact on the function. The bactericidal titers did not increase with higher antibody titers. Based on these results and on additional experiments (data not shown), the optimal percentage of CPS oxidation for all three serotypes was defined as being between 10 and 30%.

Reproductive and Developmental Toxicology Studies in Rabbits and Rats

Results from two species showed no effect of the serotype Ia, Ib and III/CRM197 conjugates on embryonic or fetal development.

In rabbits, a combination of the three conjugates with aluminum hydroxide adjuvant was administered by intramuscular injection at a clinical dose of 20/20/20 μg (based on mass of each saccharide) on days −35, −21 and −7 relative to mating on day 0 (pre-mating period) and on gestation days 7 and 20 or on gestation days 7 or 20 only. Treatment resulted in neither maternal toxicity, effects on mating nor evidence of embryo lethality, fetotoxicity or teratogenicity at any dose level.

In rats, there was similarly no maternal toxicity or evidence of effects on reproductive function and embryo-fetal development when the combination was administered, and no difference was noted between groups administered the combination in saline versus the combination in aluminum hydroxide adjuvant in 3 (during gestation only) or 6 (prior to gestation and during gestation) injections. Injections were given on days −35, −21 and −7 relative to mating on day 0 as well as on days 6, 12 and 17 of gestation or only on days 6, 12 and 17 of gestation. There was no effect on the F1 generation pup survival, clinical condition or body weight during the pre-weaning period.

Human Study (1)

This study investigated a monovalent GBS serotype Ia capsular saccharide-CRM197 conjugate vaccine. Test groups of 10 subjects were administered 1 or 2 injections at 5, 10 or 20 μg (measured as mass of saccharide) doses. Placebo groups of 3 and 2 subjects received 1 and 2 injections of saline respectively. Blood was drawn from each subject at screening and a month after the first injection for analysis by ELISA. Additionally, at 3 months into the study, the 2-injection groups had a blood draw at the time they received the second injection, and then returned a month later for another blood draw. Further blood draws were carried out at 6, 12 and 24 months after the last injection the subject had received.

The ELISA measures the concentration of specific antibodies against GBS Ia (or Ib and III in the studies described below) capsular saccharides. Microtiter plates were coated with 1 μg/ml of the appropriate GBS saccharide (conjugated to HSA) and were incubated with sera from study subjects for lh at 37° C. After 3 washes, the plates were incubated with an alkaline phosphatase (AP) labeled anti-human IgG secondary antibody for 90 min at 37° C. followed by additional 3 washes. The substrate (pNPP) was added to the plate and incubated for 30 min at room temperature. The AP catalyzes the hydrolysis of the substrate generating a colorimetric reaction which can be quantified by an ELISA reader at 405 nm (reference filter 650 nm). The evaluation of the antibody concentration was done using a standard curve. A summary of the geometric mean concentration (μg/ml) of anti-Ia antibodies for each group is given in table 16 below:

TABLE 16
Geometric mean concentrations (and geometric mean ratios) for monovalent GBS
serotype Ia capsular saccharide-CRM197 conjugate vaccine study.

| | Placebo | Placebo+ | GBS Ia 5 | GBS Ia 5+ | GBS Ia10 | GBS Ia10+ | GBS Ia20 | GBS Ia20+ |
|---|---|---|---|---|---|---|---|---|
| | N = 3 | N = 2 | N = 10 | N = 10 | N = 10 | N = 10 | N = 10 | N = 10 |
| Baseline (visit 0) | 0.84 | 0.16 | 1.05 | 0.42 | 1.25 | 0.37 | 0.54 | 0.2 |
| | (0.12-5.86) | (0.015-1.77) | (0.36-3.05) | (0.14-1.29) | (0.38-4.1) | (0.1-1.32) | (0.19-1.57) | (0.07-0.59) |
| | | | | N = 9 | N = 8 | N = 7 | | |
| 1 month after last | 0.8 | 0.16 | 43 | 5.94 | 7.94 | 14 | 25 | 6.88 |
| immunization | (0.034-19) | (0.0007-38) | (7.6-240) | (1.06-33) | (1.41-45) | (2.52-79) | (4.45-140) | (1.12-42) |
| | | N = 1 | | | | | | N = 9 |
| 1 month after last | 0.96 | 1 | 41 | 16 | 10 | 53 | 46 | 33 |
| immunization to | (0.093-9.92) | (0.018-57) | (11-146) | (4.05-60) | (2.4-42) | (12-246) | (13-166) | (8.55-127) |
| Baseline (visit 0) | | N = 1 | | N = 9 | N = 8 | N = 7 | | N = 9 |
| 6 months after last | 0.73 | 0.16 | 19 | 3.08 | 12 | 7.03 | 14 | 4.21 |
| immunization | (0.05-11) | (0.0016-17) | (4.43-83) | (0.71-13) | (2.66-50) | (1.63-30) | (3.22-60) | (0.9-20) |
| | | N = 1 | | | | | | N = 9 |
| 6 months after last | 0.87 | 1 | 18 | 7.9 | 13 | 27 | 26 | 20 |
| immunization to | (0.12-6.39) | (0.032-31) | (6.14-54) | (2.5-25) | (3.71-43) | (7.2-98) | (8.67-77) | (6.39-64) |
| Baseline (visit 0) | | N = 1 | | N = 9 | N = 8 | N = 7 | | N = 9 |
| 12 months after | 0.79 | 0.16 | 14 | 2.23 | 7.28 | 7.17 | 9.96 | 3.24 |
| last immunization | (0.054-12) | (0.0016-17) | (3.3-62) | (0.51-9.68) | (1.68-32) | (1.53-34) | (2.29-43) | (0.63-17) |
| | | N = 1 | | | | N = 9 | | N = 8 |
| 12 months after | 0.94 | 1 | 14 | 5.59 | 8.02 | 19 | 18 | 17 |
| last immunization | (0.14-6.46) | (0.036-28) | (4.76-39) | (1.84-17) | (2.46-26) | (5.49-68) | (6.43-53) | (5.31-56) |
| to | | N = 1 | | N = 9 | N = 8 | N = 7 | | N = 8 |
| Baseline (visit 0) | | | | | | | | |
| 24 months after | 0.66 | | 3.57 | 2.74 | 10 | 5.05 | 8.63 | 3.46 |
| last immunization | (0.043-10) | | (0.59-21) | (0.56-13) | (1.74-63) | (0.84-30) | (1.78-42) | (0.65-19) |
| | | | N = 7 | N = 9 | N = 7 | N = 7 | N = 9 | N = 8 |
| 24 months after | 0.79 | | 7.47 | 6.51 | 7.88 | 14 | 14 | 16 |
| last immunization | (0.11-5.95) | | (2-28) | (1.9-22) | (1.9-33) | (3.26-56) | (4.38-45) | (4.68-55) |
| to | | | N = 7 | N = 8 | N = 6 | N = 6 | N = 9 | N = 8 |
| Baseline (visit 0) | | | | | | | | |

+ two injections

Overall the GMC data show a significant increase between baseline and later timepoints (e.g. GMC range from 6 to 43 μg/ml one month after the last vaccination) and although there was a decline over time, 24 months into the study, the group GMC were still multiple-fold higher than at baseline (the GMR ranges from 7 to 14 at 24 months). Judging by the GMC point estimate, the group receiving the 5 μg dose as a single vaccination had the highest overall response at a month after the last vaccination.

The number of subjects with antibody levels ≧3 μg/mL showed similar numbers of “responders” across the different doses (11, 13 and 12 out of 20 for 5, 10 and 20 doses respectively), and different vaccination schedules (18 out of 20 for both), at a month after the last vaccination (data not shown). The percentage of subjects with antibody levels >5 μg/mL confirmed the same observations (data not shown). These cut-offs were intended to allow responses to be assessed in the context of potential serologic correlates of protection (based on ref 2). These data suggest that there is no observable contribution by either a second vaccination or a higher vaccine dose. As no dose-response was observed, it is possible that a dose of 5 μg or lower may be an optimal dose in an adult population. No sustained advantage was observed from administering 2 injections compared to 1 injection for the groups receiving 5 and 20 μg. The group receiving 10 μg dose showed higher peak responses (at 1 month post vaccination) after two vs one injection, but this trend was reversed at subsequent (steady-state) time-points.

Safety analysis was assessed based on a number of different criteria. No safety issues stood out, and no dose dependent response was noticeable.

Human Study (2)

This study investigated monovalent GBS serotype Ib and III capsular saccharide-CRM197 conjugate vaccines. Test groups of 10 subects were administered 1 or 2 injections at 5, 10 or 20 μg (measured as mass of saccharide) doses. Placebo groups of 3 and 2 subjects received 1 and 2 injections of saline respectively. Blood was drawn from each subject at screening and a month after the first injection for analysis by ELISA. Additionally, at 3 months into the study, the 2-injection groups had a blood draw at the time they received the second injection, and then returned a month later for another blood draw. Further blood draws were carried out at 6, 12 and 24 months after the last injection the subject had received. A summary of the geometric mean concentration of anti-Ib and III antibodies for each group is given in table 17 below.

TABLE 17
Geometric mean concentrations (and geometric mean ratios) for monovalent GBS
serotype III and Ib capsular saccharide-CRM197 conjugate vaccines study.

| | | Placebo | Placebo+ | GBS Ib 5 | GBS Ib 5+ |
|---|---|---|---|---|---|
| | | N = 2 | N = 2 | N = 8 | N = 10 |
| GBS Ib | Baseline (visit 0) | 0.1 | 0.042 | 0.27 | 0.24 |
| | | (0.0042-2.38) | (0.0017-0.99) | (0.066-1.13) | (0.08-0.75) |
| | | N = 1 | N = 1 | N = 5 | N = 8 |
| | 1 month after last | 0.3 | 0.53 | 1.89 | 11 |
| | immunization | (0.0019-47) | (0.015-19) | (0.32-11) | (1.77-63) |
| | | N = 1 | | | N = 8 |
| | 1 month after last | | 9.64 | 60 | 47 |
| | immunization to | | (0.26-359) | (9.78-364) | (11-208) |
| | Baseline (visit 0) | | N = 1 | N = 4 | N = 6 |
| | 6 months after last | 0.091 | 0.3 | 1.53 | 7.52 |
| | immunization | (0.0051-1.63) | (0.0051-18) | (0.36-6.48) | (2.07-27) |
| | | | N = 1 | | |
| | 6 months after last | 0.42 | | 32 | 36 |
| | immunization to | (0.031-5.64) | | (8.56-116) | (14-91) |
| | Baseline (visit 0) | N = 1 | | N = 4 | N = 8 |
| | 12 months after last | 0.091 | 0.4 | 1.29 | 5.77 |
| | immunization | (0.0054-1.53) | (0.0074-22) | (0.31-5.26) | (1.53-22) |
| | | | N = 1 | | N = 9 |
| | 12 months after last | 0.42 | | 22 | 29 |
| | immunization to | (0.036-4.85) | | (6.52-76) | (12-75) |
| | Baseline (visit 0) | N = 1 | | N = 4 | N = 7 |
| | 24 months after last | 0.091 | 0.1 | 1.34 | 3.8 |
| | immunization | (0.004-2.07) | (0.0012-8.29) | (0.28-6.4) | (0.94-15) |
| | | | N = 1 | | |
| | 24 months after last | | | 27 | 19 |
| | immunization to | | | (6.49-111) | (7.07-53) |
| | Baseline (visit 0) | | | N = 4 | N = 8 |
| | | N = 3 | N = 2 | N = 8 | N = 10 |
| GBS III | Baseline (visit 0) | 0.034 | 0.27 | 0.27 | 1.64 |
| | | (0.0007-1.55) | (0.018-3.98) | (0.057-1.3) | (0.46-5.86) |
| | | N = 1 | | N = 6 | N = 9 |
| | 1 month after last | 0.14 | 0.88 | 13 | 31 |
| | immunization | (0.0099-2.11) | (0.033-24) | (2.44-65) | (7.07-134) |
| | 1 month after last | 8.82 | 3.31 | 44 | 31 |
| | immunization to | (0.64-122) | (0.51-21) | (15-130) | (12-84) |
| | Baseline (visit 0) | N = 1 | | N = 6 | N = 7 |
| | 6 months after last | 0.034 | 1.9 | 4.97 | 21 |
| | immunization | (0.0005-2.11) | (0.031-118) | (1.04-24) | (5.57-76) |
| | | N = 1 | N = 1 | N = 7 | |
| | 6 months after last | | 0.9 | 23 | 22 |
| | immunization to | | (0.086-9.48) | (8.85-60) | (8.87-52) |
| | Baseline (visit 0) | | N = 1 | N = 6 | N = 7 |
| | 12 months after last | 0.1 | 2 | 3.7 | 16 |
| | immunization | (0.01-1.02) | (0.036-110) | (0.81-17) | (4.39-55) |
| | | | N = 1 | N = 7 | |
| | 12 months after last | 8.82 | 0.95 | 18 | 17 |
| | immunization to | (0.75-104) | (0.081-11) | (6.41-48) | (6.51-42) |
| | Baseline (visit 0) | N = 1 | N = 1 | N = 6 | N = 7 |
| | 24 months after last | 0.034 | 0.4 | 0.33 | 13 |
| | immunization | (0.0008-1.39) | (0.029-5.51) | (0.039-2.81) | (3.92-41) |
| | | N = 1 | | N = 3 | |
| | 24 months after last | 1 | 1.5 | 2.45 | 14 |
| | immunization to | (0.092-11) | (0.28-8.1) | (0.45-13) | (5.57-34) |
| | Baseline (visit 0) | N = 1 | | N = 2 | N = 7 |

| | | GBS Ib10 | GBS Ib10+ | GBS Ib20 | GBS Ib20+ |
|---|---|---|---|---|---|
| | | N = 9 | N = 11 | N = 9 | N = 10 |
| GBS Ib | Baseline (visit 0) | 0.088 | 0.44 | 0.38 | 0.2 |
| | | (0.021-0.36) | (0.16-1.21) | (0.11-1.25) | (0.074-0.55) |
| | | N = 5 | N = 10 | N = 7 | |
| | 1 month after last | 2.63 | 18 | 14 | 10 |
| | immunization | (0.49-14) | (3.82-81) | (2.6-76) | (2.11-52) |
| | 1 month after last | 46 | 48 | 68 | 56 |
| | immunization to | (9.13-232) | (15-152) | (17-267) | (17-186) |
| | Baseline (visit 0) | N = 5 | N = 10 | N = 7 | N = 9 |
| | 6 months after last | 5.45 | 11 | 6.23 | 9.2 |
| | immunization | (1.4-21) | (3.09-36) | (1.6-24) | (2.53-33) |
| | 6 months after last | 97 | 28 | 26 | 47 |
| | immunization to | (30-310) | (12-63) | (9.61-69) | (20-112) |
| | Baseline (visit 0) | N = 5 | N = 10 | N = 7 | N = 9 |
| | 12 months after last | 3.24 | 9.52 | 4.98 | 6.67 |
| | immunization | (0.79-13) | (2.86-32) | (1.32-19) | (1.89-24) |
| | | N = 8 | | | |
| | 12 months after last | 45 | 26 | 23 | 34 |
| | immunization to | (13-152) | (12-57) | (8.96-57) | (15-78) |
| | Baseline (visit 0) | N = 4 | N = 10 | N = 7 | N = 9 |
| | 24 months after last | 2.58 | 9.49 | 3.22 | 3.92 |
| | immunization | (0.59-11) | (2.35-38) | (0.53-20) | (0.9-17) |
| | | | N = 10 | N = 6 | N = 9 |
| | 24 months after last | 53 | 25 | 15 | 22 |
| | immunization to | (15-188) | (9.65-64) | (3.55-61) | (7.95-59) |
| | Baseline (visit 0) | N = 5 | N = 9 | N = 4 | N = 8 |
| | | N = 10 | N = 9 | N = 9 | N = 10 |
| GBS III | Baseline (visit 0) | 0.23 | 0.65 | 0.89 | 1.93 |
| | | (0.065-0.83) | (0.18-2.34) | (0.21-3.77) | (0.58-6.47) |
| | | N = 9 | | N = 7 | |
| | 1 month after last | 2.72 | 31 | 22 | 22 |
| | immunization | (0.63-12) | (6.64-147) | (4.73-105) | (5.03-95) |
| | 1 month after last | 10 | 42 | 63 | 14 |
| | immunization to | (4.26-25) | (16-106) | (23-172) | (5.94-34) |
| | Baseline (visit 0) | N = 9 | N = 8 | N = 7 | N = 9 |
| | 6 months after last | 2.7 | 19 | 18 | 15 |
| | immunization | (0.73-9.95) | (4.89-77) | (4.43-69) | (3.84-60) |
| | | | | | N = 9 |
| | 6 months after last | 11 | 25 | 33 | 9.86 |
| | immunization to | (5-24) | (11-58) | (14-80) | (4.3-23) |
| | Baseline (visit 0) | N = 9 | N = 8 | N = 7 | N = 8 |
| | 12 months after last | 2.56 | 15 | 13 | 13 |
| | immunization | (0.72-9.09) | (3.97-57) | (3.35-48) | (3.05-52) |
| | | | | | N = 8 |
| | 12 months after last | 10 | 20 | 22 | 9.54 |
| | immunization to | (4.61-24) | (8.35-48) | (8.81-57) | (3.76-24) |
| | Baseline (visit 0) | N = 9 | N = 8 | N = 7 | N = 7 |
| | 24 months after last | 0.71 | 24 | 11 | 6.15 |
| | immunization | (0.14-3.73) | (4.62-127) | (2.11-58) | (1.66-23) |
| | | N = 5 | N = 5 | N = 5 | N = 8 |
| | 24 months after last | 6.55 | 23 | 13 | 6.21 |
| | immunization to | (2.25-19) | (8.06-68) | (4.39-37) | (2.52-15) |
| | Baseline (visit 0) | N = 5 | N = 5 | N = 5 | N = 7 |

+ two injections

Once again, no significant dose-response or advantage from administering two injections compared to one was observed in this small study.

Safety analysis was assessed based on a number of different criteria. No safety issues stood out, and no dose dependent response was noticeable. Among the reactogenicity indicators, pain on injection site (15 occurrences out of 98 injections for serotype Ib, and 14 occurrences out of 96 injections for serotype III) was the most common complaint in the solicited local reactions for both serotypes, and headache was the one in the solicited systemic reactions, but no obvious differences between placebo and the vaccinated individuals were observed.

Human Study (3)

This study investigated a trivalent GBS serotype Ia, Ib and III capsular saccharide-CRM197 conjugate vaccine in healthy, non-pregnant women. Two different vaccine formulations were studied, each combining the three saccharides in equal proportions. Two different doses (5 μg and 20 μg, measured as mass of each saccharide, in 0.5 ml) were tested with and without alum adjuvant. The study also evaluated intramuscular 1- and 2-injection (30 days apart) schedules for each formulation. The vaccine also included 4.5 mg sodium choloride, 0.34 mg potassium dihydrogen phosphate and 7.5 mg mannitol. The study groups are summarised in Table 18 below. A placebo group (two 0.9% saline injections, 30 days apart) with 20 subjects was also tested.

TABLE 18
Study groups for trivalent GBS serotype Ia, Ib and III capsular saccharide-
CRM197 conjugate vaccine study

| | |
|---|---|
| 1 injection | 2 injections |

| Variables | 5/5/5 μg | 20/20/20 μg | 5/5/5 μg | 20/20/20 μg |
|---|---|---|---|---|
| No alum | N = 40 | N = 39 | N = 40 | N = 40 |
| Alum | N = 40 | N = 39 | N = 40 | N = 40 |

Blood was drawn from each subject at screening and a month after the first injection for analysis of immunogenicity by ELISA. The 2-injection groups received the second injection after the blood draw at the one-month timepoint. Blood was also drawn from all groups at 3 months into the study. A summary of the geometric mean concentration of anti-Ia, Ib and III antibodies for each group (adjusted for baseline antibody concentrations and excluding the placebo group) is given in table 19 below.

TABLE 19
Geometric mean concentrations (and geometric mean ratios) for trivalent GBS
serotype Ia, Ib and III capsular saccharide-CRM197 conjugate vaccine study.

| | | 5 na | 5+ na | 20 na | 20+ na | 5 adv | 5+ adv | 20 adv | 20+ adv |
|---|---|---|---|---|---|---|---|---|---|
| | | N = 40 | N = 40 | N = 38 | N = 39 | N = 40 | N = 40 | N = 39 | N = 39 |
| GBS | Screening | 0.71 | 0.65 | 0.48 | 0.49 | 0.57 | 0.71 | 0.59 | 0.45 |
| Ia | | (0.41-1.21) | (0.37-1.16) | (0.27-0.85) | (0.28-0.87) | (0.32-1.01) | (0.4-1.27) | (0.33-1.05) | (0.26-0.79) |
| | | | N = 35 | N = 35 | N = 37 | N = 36 | N = 35 | N = 35 | N = 37 |
| | Day 31 | 13 | 12 | 20 | 18 | 16 | 8.26 | 12 | 8.88 |
| | | (7.06-25) | (5.99-23) | (10-41) | (9.41-36) | (8.02-30) | (4.19-16) | (6.12-24) | (4.57-17) |
| | | | | | | | N = 39 | | |
| | Day 31 to | 23 | 20 | 34 | 31 | 27 | 14 | 21 | 15 |
| | Screening | (12-44) | (10-40) | (17-66) | (16-61) | (14-52) | (7.32-28) | (11-41) | (7.62-28) |
| | | | N = 35 | N = 34 | N = 36 | N = 36 | N = 35 | N = 35 | N = 37 |
| | Day 61 | 16 | 15 | 18 | 23 | 18 | 12 | 13 | 11 |
| | | (9.34-28) | (8.63-27) | (10-33) | (13-40) | (10-32) | (6.79-22) | (7.3-23) | (6.01-19) |
| | | | | | | | | | N = 38 |
| | Day 61 to | 28 | 26 | 31 | 39 | 31 | 20 | 23 | 18 |
| | Screening | (16-47) | (15-47) | (17-55) | (22-69) | (18-55) | (11-36) | (13-40) | (10-32) |
| | | | N = 35 | N = 34 | N = 36 | N = 36 | N = 35 | N = 35 | N = 36 |
| | | N = 39 | N = 40 | N = 38 | N = 39 | N = 40 | N = 40 | N = 39 | N = 37 |
| GBS | Screening | 0.15 | 0.12 | 0.1 | 0.12 | 0.14 | 0.11 | 0.12 | 0.081 |
| Ib | | (0.091-0.25) | (0.072-0.2) | (0.063-0.17) | (0.072-0.2) | (0.082-0.22) | (0.068-0.19) | (0.074-0.21) | (0.048-0.14) |
| | | N = 37 | N = 34 | N = 36 | N = 37 | N = 37 | N = 34 | N = 34 | N = 34 |
| | Day 31 | 4.92 | 4.21 | 4.59 | 4.12 | 3.94 | 3.25 | 3.31 | 2.87 |
| | | (2.67-9.06) | (2.24-7.91) | (2.51-8.41) | (2.23-7.6) | (2.15-7.23) | (1.73-6.1) | (1.74-6.28) | (1.48-5.6) |
| | | N = 38 | N = 39 | | N = 35 | N = 39 | N = 39 | N = 37 | N = 36 |
| | Day 31 to | 45 | 35 | 35 | 33 | 34 | 27 | 30 | 19 |
| | Screening | (24-85) | (18-69) | (19-67) | (17-63) | (18-64) | (14-53) | (15-59) | (9.43-37) |
| | | N = 36 | N = 33 | N = 36 | N = 35 | N = 36 | N = 33 | N = 32 | N = 31 |
| | Day 61 | 5.17 | 5.29 | 4.69 | 5.35 | 4.1 | 4.09 | 3.74 | 3.39 |
| | | (3.17-8.45) | (3.19-8.78) | (2.84-7.73) | (3.26-8.76) | (2.52-6.68) | (2.47-6.79) | (2.26-6.22) | (1.98-5.8) |
| | Day 61 to | 47 | 46 | 38 | 45 | 35 | 35 | 33 | 24 |
| | Screening | (29-78) | (27-77) | (23-64) | (27-75) | (21-59) | (21-59) | (20-56) | (14-41) |
| | | N = 37 | N = 34 | N = 35 | N = 36 | N = 37 | N = 34 | N = 34 | N = 32 |
| | | N = 34 | N = 36 | N = 38 | N = 36 | N = 35 | N = 36 | N = 35 | N = 36 |
| GBS | Screening | 0.3 | 0.16 | 0.17 | 0.14 | 0.18 | 0.38 | 0.15 | 0.24 |
| III | | (0.16-0.54) | (0.088-0.29) | (0.096-0.3) | (0.078-0.25) | (0.1-0.34) | (0.21-0.69) | (0.082-0.27) | (0.13-0.43) |
| | | | N = 34 | | | N = 34 | | | |
| | Day 31 | 7.82 | 5.48 | 8.13 | 8.31 | 5.5 | 5.36 | 8.51 | 6.03 |
| | | (4.24-14) | (3.03-9.91) | (4.66-14) | (4.47-15) | (3-10) | (2.88-10) | (4.45-16) | (3.41-11) |
| | | | | N = 37 | N = 34 | N = 34 | N = 34 | N = 31 | |
| | Day 31 to | 34 | 25 | 36 | 34 | 26 | 24 | 35 | 29 |
| | Screening | (19-63) | (14-45) | (21-64) | (19-62) | (14-47) | (14-44) | (19-66) | (16-51) |
| | | N = 31 | N = 32 | N = 36 | N = 31 | N = 30 | N = 32 | N = 28 | N = 34 |
| | Day 61 | 7.5 | 8.71 | 8.48 | 9.59 | 5.35 | 7.75 | 8.23 | 7.79 |
| | | (4.43-13) | (5.31-14) | (5.26-14) | (5.72-16) | (3.2-8.93) | (4.58-13) | (4.77-14) | (4.74-13) |
| | | N = 33 | | N = 37 | N = 35 | | | N = 32 | N = 35 |
| | Day 61 to | 33 | 43 | 39 | 43 | 26 | 33 | 38 | 38 |
| | Screening | (20-55) | (26-70) | (25-64) | (26-71) | (15-43) | (20-54) | (22-64) | (23-62) |
| | | N = 31 | N = 34 | N = 36 | N = 33 | N = 31 | N = 33 | N = 29 | N = 33 |

+ two injections
na—no adjuvant
adv—with adjuvant

The vaccine was immunogenic, inducing in between 80% and 100% of the subjects at least a 2-fold increase in GBS specific antibodies across the different serotypes. A comparison of the GMCs from the eight groups revealed a) no contribution from a second injection compared to a single injection only; b) no contribution from the inclusion of alum adjuvant compared to no adjuvant; and c) no contribution from the higher dose of 20/20/20 μg versus 5/5/5 μg.

More specifically, there was no consistent increase in antibody response among subjects receiving two vaccine injections compared to those receiving only one vaccine injection against any of the GBS serotypes (Ia, Ib or III). This lack of contribution of the second vaccine injection was observed regardless of the dose (5/5/5 or 20/20/20 μg) or the formulation (no alum or alum adjuvant). For GBS Ia, GMC measurements for each of the eight groups ranged from 7 to 20 μg/ml on day 61 of the study. From these results, no contribution of two injections (GMC range [7-16 μg/ml]) was observed compared to one injection (GMC range [9-20 μg/ml] (95% CI all overlapping)). Moreover, the ratio of one vs two injections was 1.2 [95% CI (0.7, 2.0)]. This result indicates practical equivalence of one vs two injections (p-value=0.5). For GBS Ib, GMC measurements for each of the eight groups ranged from 2-7 μg/ml on day 61 of the study. No contribution of two injections (GMC range [2-5 μg/ml]) was observed compared to one injection (GMC range [4-7 μg/ml] (95% CI all overlapping)). This time, the ratio of one vs two injections was 1.2 [95% CI (0.7, 2.0)]. Once again, this result indicates practical equivalence (p-value=0.5). For GBS III, measurements for each of the eight groups ranged from 5-13 μg/ml on day 61 of the study. No contribution of two injections (GMC range [5-11 μg/ml]) was observed compared to one injection (GMC range [5-13 μg/ml] (95% CI all overlapping)). The ratio of one vs two injections was 0.94 [95% CI (0.55, 1.66], indicating equivalence (p-value=0.8).

Similarly, there was no added contribution to GMC from the inclusion of alum compared to no alum. This lack of contribution of alum adjuvant was observed regardless of the dose (5/5/5 or 20/20/20 μg) or the injection number and was seen across all three serotypes (Ia, Ib and III). For GBS Ia, the GMC across the eight groups ranged from 7-20 μg/ml on day 61 of the study and showed no contribution of alum (GMC range [7-15 μg/ml] compared to no alum (GMC range [13-16 μg/ml] (95% CI all overlapping)). The ratio of group GMC for the no alum group compared to the alum group was 1.6 [95% CI (0.9, 2.6)], which suggests that the response without alum is potentially higher relative to the vaccine formulation with alum (p-value=0.11). For GBS Ib, GMC ranged from 2-7 μg/ml on day 61 of the study and showed no contribution of alum (GMC range [2-4 μg/ml] compared to no alum (GMC range [4-7 μg/ml] (95% CI all overlapping)). The ratio of group GMC for the no alum group compared to the alum group was 1.4 [95% CI (0.8, 2.4)] implying near equivalence in GMC values (p-value=0.2). For GBS III, GMC ranged from 5-13 82 g/ml on day 61 of the study and showed no contribution of alum (GMC range [5-11 μg/ml] compared to no alum (GMC range [5-13 μg/ml] (95% CI all overlapping)). The ratio of group GMC for the no alum group compared to the alum group was 1.09 [95% CI (0.6, 1.9)] implying near equivalence in GMC values (p-value=0.7).

Finally, the data allows an evaluation of the two doses (5 vs 20 μg of each of the three saccharides in the conjugates). The results suggest that the higher dose (20 μg) does not induce a higher antibody response. In particular, the ratios of GMC for subjects receiving 5 μg (across all groups) and subjects receiving 20 μg (across all groups) are 1.2 [95% CI (0.7, 2.1)] for GBS Ia; 0.7 [95% CI (0.4, 1.2)] for GBS Ib and 1.4 [95% CI (0.9, 2.5)] for GBS III. These ratios are close to 1 and the p-values of the statistical test for equality to 1, are >0.15 for all three serotypes, suggesting no discernable differences in the level of induced antibodies between the two dose regimens.

Safety was measured by the incidence of local and systemic reactogenicity, adverse events and serious adverse events, as well as clinical laboratory results. The trivalent GBS vaccine was found safe and well tolerated in all of the eight vaccine study groups when compared to placebo.

Safety was evaluated by: percentages of subjects with solicited local (i.e injection site pain, ecchymosis, erythema, induration, and swelling) and solicited systemic (i.e. chills, nausea, malaise, myalgia, headache, fatigue, arthralgia, rash, fever [defined as axillary temperature ≧38° C.], and other) reactions occurring during the 7 days following each vaccination together with severity of reactions; all of other adverse events reported from day 1 to day 23 after each vaccination; percentages of subjects with reported serious adverse events and/or adverse events resulting in withdrawal from the study, per vaccine group for up to Day 61.

Human Study (4)

The responses of subjects with antibody (Ab) levels below detection at study entry (0.4, 0.084 and 0.068 μg/ml for serotypes Ia, Ib and III respectively) were of particular interest. This subset analysis was carried out on the data from Human study (3) above. For each serotype, data were assessed as:
(a) GMC for each injection/formulation/dose group, and the corresponding 95% CI
(b) GMC over all subjects receiving (i) 1 injection regardless of a group assignment and this was compared to the GMC of all subjects receiving 2 injections. Similarly, the GMC of subjects receiving no adjuvant compared to the GMC of subjects receiving alum, as well as GMC receiving 5/5/5 μg dose compared to the GMC of all subjects receiving 20/20/20 μg. The assessment was based on the ratio of GMC, together with the two-sided 95% CI around the calculated ratio.
(c) Proportion of subjects with at least 4-fold change from baseline, assumed as half the lowest level of detection (lld)

In general, approximately 25% and 50% of women presented with Ab levels below the limit of detection for serotypes III and la/lb respectively. The percentage of subjects in this subset achieving ≧4-fold increase in Ab level at day 61 compared to baseline (where baseline value is assigned half lld) range from 64-95% (serotype Ia), 80-100% (serotype III) and 81-100% (serotype Ib).

Similarly to results from the full study cohort, subjects with undetectable Ab levels at study entry also fail to show additional benefit from 2 injections (vs 1 injection), from a higher dosage (vs lower dosage) or from inclusion of alum (vs no adjuvant). The ratio of GMC (on day 61) for all 1 injection vs all 2 injection subjects was 1.1 (0.6-1.8; serotype Ia), 0.7 (0.3-1.5; serotype III) and 0.9 (0.5-1.4; serotype Ib); for all 5 μg vs. all 20 μg dosage subjects was 1.3 (0.8-2.1; serotype Ia), 1.4 (0.7-2.8; serotype III) and 1.4 (0.9-2.3; serotype Ib); for all no adjuvant subjects vs all alum subjects was 1.4 (0.8-2.4; serotype Ia), 1 (0.5-2.0; serotype III) and 1.7 (1.1-2.7; serotype Ib)

Mouse Study (15)

Mice were primed with CRM197 and aluminium hydroxide adjuvant or aluminium hydroxide adjuvant alone at day 0 and then immunized with a GBS serotype III/CRM197 conjugate with or without the adjuvant aluminium hydroxide adjuvant at days 21 and 35. Blood was drawn on day 0 and before vaccination on days 21 and 35. IgG/IgM serum titers to the GBS serotype III polysaccharide and CRM197 carrier protein were measured from the blood samples.

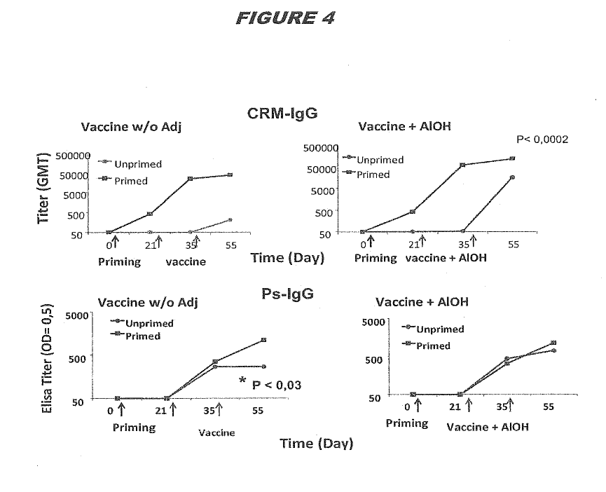

As shown in FIG. 4, priming with the CRM197 carrier resulted in a significantly higher IgG antibody response to the carrier after one and two doses of the vaccine (with or without adjuvant) compared to unprimed mice (P<0.0002). Priming also resulted in a good antibody response against the GBS serotype III polysaccharide after two doses of vaccine (with or without adjuvant). Unprimed mice required the adjuvant in order to reach an anti-polysaccharide antibody titer comparable to that observed in primed mice. In unprimed mice, when the glycoconjugate vaccine was administered without adjuvant, the antibody titer was significantly lower than in the other groups (P<0.03).

Priming with CRM197 therefore seems to have a positive influence on the subsequent antibody response to the GBS capsular saccharide component of the conjugate, even when administered without an adjuvant.

Rat and Rabbit Studies

Studies to assess potential reproductive and developmental toxicity of the trivalent GBS serotype Ia, Ib and III capsular saccharide-CRM197 conjugate vaccine were carried out in rats and rabbits.

The rat study was carried out according to table 20 below:

TABLE 20
Rat study

| | | | | Number of animals | |
|---|---|---|---|---|---|
| | Dose each | SC dose | Dosing days | C section | |
| | antigen^{±} | volume | relative to | (gestation day | |
| Treatment | (μg) | (mL) | mating on Day 0 | 21) | Natural delivery |
| Control | 0/0/0 | 0.5 | −35, −21, −7, | 24 | 24 |
| (saline) | | | 6, 12, 17 | | |
| GBS vaccine | 20/20/20 | 0.5 | | 24 | 24 |
| GBS vaccine | 20/20/20 | 0.5 | 6, 12, 17 | 24 | 24 |
| GBS vaccine + | 20/20/20 | 0.5 | −35, −21, −7, | 24 | 24 |
| alum* | | | 6, 12, 17 | | |
| GBS vaccine + | 20/20/20 | 0.5 | 6, 12, 17 | 24 | 24 |
| alum* | | | | | |

^{±}to serotype Ia/Ib/III
*aluminum hydroxide, 2 mg/mL

Subcutaneous administration of the trivalent vaccine to female rats on study days 1, 15, 29 (premating period) and/or on gestation days 6, 12 and 17 at a dose of 20 μg with or without aluminum hydroxide resulted in no maternal toxicity or effects on reproductive function or embryofetal development. No differences were noted between groups treated with three or six injections of the trivalent vaccine with or without aluminum hydroxide adjuvant. Additionally, there was no effect on the F1 generation pup survival, clinical condition or body weight or reproductive ability.

The rabbit study was carried out according to table 21 below:

TABLE 21
Rabbit study

| | | | | Number of animals | |
|---|---|---|---|---|---|
| | Dose each | IM dose | Dosing days | C section | |
| | antigen^{±} | volume | relative to | (gestation day | Natural |
| Treatment | (μg) | (mL) | mating on Day 0 | 29) | delivery |
| Control | 0/0/0 | 0.5 | −35, −21, −7, 7, 20 | 23 | 25 |
| (saline) | | | | | |
| GBS vaccine + | 20/20/20 | 0.5 | −35, −21, −7, 7, 20 | 23 | 25 |
| alum* | | | | | |
| GBS vaccine + | 20/20/20 | 0.5 | 7, 20 | 23 | 25 |
| alum | | | | | |

^{±}to serotype Ia/Ib/III
*aluminum hydroxide, 2 mg/mL

Intramuscular administration of the trivalent vaccine plus aluminum hydroxide to female rabbits, at a dose of 20 μg on study days 1, 15 and 29 (premating period) and/or on gestation days 7 and 20, resulted in neither maternal toxicity, effects on mating nor evidence of embryolethality, fetotoxicity or teratogenicity. There were no differences between the adult F1 generation of control and vaccine-treated does.

These studies showed that the trivalent vaccine was immunogenic and did not have any prenatal or postnatal effects on pregnant rats or rabbits or their offspring.

Stabilty Study

The stability of the trivalent GBS serotype Ia, Ib and III capsular saccharide-CRM197 conjugate vaccine was measured during 1 month of storage at two different temperatures. The vaccine was formulated by pooling the three glycoconjugates, each one present at 80 μg saccharide/ml in 10 mM KH₂PO₄ and 3% mannitol. 3-ml single dose vials were filled with 0.3 ml of solution, partially capped with bromobuthyl siliconized rubber stopper and submitted to a freeze-drying cycle. Once the lyophilization process was over, the vials were stored at 2-8° C. or 36-38° C. A slight increase in free saccharide content was detected (using HPAEC-PAD) upon storage at 36-38° C. However, overall the trivalent vaccine was stable upon storage up to one month at both 2-8° C. and at 36-38° C.

It will be understood that the invention has been described by way of example only and modifications may be made whilst remaining within the scope and spirit of the invention.

### REFERENCES

[1] Baltimore et al. (1977) J Immunol.118(2):673-8

[2] Lin et al. (2001) J Infect Dis. 184(8):1022-8.

1. A method for immunizing a human patient against infection by Streptococcus agalactiae, comprising administering to the patient a human unit dose of an immunogenic composition comprising:
(a) a first conjugate that is a capsular saccharide from Streptococcus agalactiae (GBS) serotype Ia conjugated to a first carrier protein, wherein the first carrier protein is CRM197;
(b) a second conjugate that is a capsular saccharide from GBS serotype Ib conjugated to a second carrier protein, wherein the second carrier protein is CRM197; and
(c) a third conjugate that is a capsular saccharide from GBS serotype III conjugated to a third carrier protein, wherein the third carrier protein is CRM197,
wherein (i) each GBS capsular saccharide is present at an amount of about 5 μg, 10 μg, or 20 μg per human unit dose of the immunogenic composition and (ii) the immunogenic composition does not contain an adjuvant.

2. The method of claim 1, wherein the the patient is a female of child-bearing age.

3. The method of claim 2, wherein the patient is pregnant.
